(19) United States
(12) Patent Application Publication
(10) Pub. No.: US 2025/0288599 A1
(43) Pub. Date: Sep. 18, 2025

(54) PHARMACEUTICAL COMPOSITIONS AND METHODS FOR TREATING OSTEOARTHRITIS

(71) Applicant: INSIGNIA PHARMACEUTICALS, LLC, Wilmington, DE (US)

(72) Inventors: David BAR-OR, Englewood, CO (US); Gregory THOMAS, Englewood, CO (US); Melissa HAUSBURG, Englewood, CO (US); Jason WILLIAMS, Englewood, CO (US); Raphael BAR-OR, Englewood, CO (US)

(73) Assignee: INSIGNIA PHARMACEUTICALS, LLC, Wilmington, DE (US)

(21) Appl. No.: 19/079,370

(22) Filed: Mar. 13, 2025

Related U.S. Application Data
(60) Provisional application No. 63/564,880, filed on Mar. 13, 2024.

(51) Int. Cl. A61K 31/573; A61K 9/00; A61K 9/06; A61K 31/20; A61K 31/385; A61K 31/405; A61K 47/36; A61P 19/02

(52) U.S. Cl. CPC A61K 31/573; A61K 9/0019; A61K 9/06; A61K 31/20; A61K 31/385; A61K 31/405; A61K 47/36; A61P 19/02

(57) ABSTRACT

A pharmaceutical composition is provided. The pharmaceutical composition includes dexamethasone at a concentration of at least 400 picomolar and up to 20 millimolar and decanoic acid at a concentration of at least 10 micromolar and up to 5 millimolar. In some embodiments, the pharmaceutical composition is used to treat osteoarthritis.

### CROSS-REFERENCE TO RELATED APPLICATIONS

This patent application claims priority of U.S. Provisional Patent Application No. 63/564,880, entitled “PHARMACEUTICAL COMPOSITIONS AND METHODS FOR TREATING OSTEOARTHRITIS,” filed Mar. 13, 2024, which is hereby incorporated by reference in its entirety.

### BACKGROUND

Osteoarthritis is the most common degenerative joint disease affecting 25 to 35 million people in the United States alone. While once thought to be a non-inflammatory “wear and tear” disease, it is now recognized that inflammation within articular joints drives the disease, resulting in chronic pain and disability that worsens with age. For example, symptomatic osteoarthritis of the knee occurs in over ten percent of persons aged 60 and above, and knee osteoarthritis decreases mobility more than any other medical condition in seniors.

Current pharmaceutical interventions for osteoarthritis treat inflammation and pain, but the interventions present limiting or otherwise detrimental side effects. Repeated injections of corticosteroids at high doses, for example, can cause severe side effects such as cartilage loss and bone fractures.

In view of these pharmaceutical limitations, chronic osteoarthritis often results in progressive disability that eventually requires total joint replacement. The increased prevalence of osteoarthritis in aging and obese populations suggests a growing clinical need for safe and effective pharmaceutical interventions to delay and potentially eliminate the need for orthopedic surgery.

### SUMMARY

Various aspects of this disclosure are related to pharmaceutical compositions comprising varying concentrations of a lipid selected from medium-chain fatty acids and carboxylates and monoglycerides thereof, and a corticosteroid, alone or in combination with other chemicals and additives (e.g., tryptophan (Try), hyaluronic acid (HA), lactate (Lac), water).

Various aspects of this disclosure are related to pharmaceutical compositions comprising a lipid selected from medium-chain fatty acids and carboxylates and monoglycerides thereof, and a corticosteroid, such as dexamethasone (Dex) or ester thereof. In some embodiments, the lipid is polysaturated. In some specific embodiments, the lipid is a medium-chain fatty acid selected from hexanoic acid (caproic acid), octanoic acid (caprylic acid), decanoic acid (DA) (capric acid), and dodecanoic acid (lauric acid); the lipid is a carboxylate selected from hexanoate (caproate), octanoate (caprylate), decanoate (caprate), and dodecanoate (laurate); or the lipid is a monoglyceride selected from monohexanoin (monocaproin), monooctanoin (monocaprylin), monodecanoin (monocaprin), and monododecanoin (monolaurin). This disclosure teaches that the foregoing lipids and corticosteroids generally display anti-inflammatory properties that are particularly relevant to osteoarthritis and advantageously display synergy when combined with corticosteroids that are commonly used to treat osteoarthritis.

Various aspects of this disclosure are also related to pharmaceutical compositions comprising a lipid selected from medium-chain fatty acids and carboxylates and monoglycerides thereof, a corticosteroid, such as Dex or ester thereof, and Lac such as Ringer's lactate (LR). In some embodiments, the lipid is polysaturated. In some specific embodiments, the lipid is a medium-chain fatty acid selected from hexanoic acid (caproic acid), octanoic acid (caprylic acid), DA (capric acid), and dodecanoic acid (lauric acid); the lipid is a carboxylate selected from hexanoate (caproate), octanoate (caprylate), decanoate (caprate), and dodecanoate (laurate); or the lipid is a monoglyceride selected from monohexanoin (monocaproin), monooctanoin (monocaprylin), monodecanoin (monocaprin), and monododecanoin (monolaurin). This disclosure teaches that the foregoing lipids, corticosteroids, and Lac generally display anti-inflammatory properties that are particularly relevant to osteoarthritis and advantageously display synergy when combined with corticosteroids that are commonly used to treat osteoarthritis.

Without limiting this disclosure or any patent claim that issues therefrom, the pharmaceutical compositions of this disclosure generally display synergy between the corticosteroid and the lipid at reducing inflammation generally and inflammasome-mediated inflammation specifically. The pharmaceutical compositions also generally display synergy between the corticosteroid and the lipid at reducing collagenase activity generally and matrix metalloproteinase 13 (MMP13; collagenase 3) specifically, and thus, the pharmaceutical compositions may be effective at reducing the loss of cartilage in conditions such as osteoarthritis. The pharmaceutical compositions also generally display synergy between the corticosteroid and the lipid at redifferentiating chondrocytes that display a fibroblast-like phenotype into normal chondrocytes, and thus, the pharmaceutical compositions may be effective at reversing conditions that display deleterious effects on cartilage such as osteoarthritis.

Aqueous dexamethasone phosphate (Dxp) solutions for injection generally comprise several milligrams of Dxp per milliliter of the formulation (for example, 3.3 milligrams per milliliter or 4 milligrams per milliliter), which is generally around 5 to 10 millimolar Dxp. The experimental results set forth below suggest that the addition of a medium-chain fatty acid (such as DA) to an aqueous Dxp formulation may allow for either a reduced concentration of the Dex, a reduced volume of Dex to be administered, or both. Importantly, the concentration of DA necessary to display a synergistic effect in combination with a corticosteroid was determined to be on the order of 10 micromolar to 1 millimolar, which is less than the solubility of DA in water.

The results also demonstrate that Lac displays a beneficial effect on inflammation, collagen production, and the re-differentiation of chondrocytes as assessed by prostaglandin E₂ (PGE₂) release, the transcription of type II collagen (Col2a1), and the transcription of SRY-box transcription factor 9 (SOX9), respectively. These effects were observed in formulations comparable to LR. An aqueous Dxp solution for injection might therefore be formulated in LR with the addition of 10 micromolar to 1 millimolar DA, for example, to result in an improved formulation that can reduce the overall amount of Dxp to be administered thereby reducing the risk of side effects that confound the treatment of osteoarthritis and other conditions by corticosteroid injection.

In some embodiments, the corticosteroid and the lipid are covalently bound. The corticosteroid may be, for example, a Dex ester that is covalently bound, for example to hexanoic acid, octanoic acid, DA, or dodecanoic acid. In some specific embodiments, the pharmaceutical composition comprises Dex-21 hexanoate, Dex-21 octanoate, Dex-21 decanoate, or Dex-21 dodecanoate. Other Dex esters are known and include, for example, Dex-21 palmitate and Dex-21 linoleate. Esters or ethers of other corticosteroids such as cortisol-21 octanoate and cortisol-21 decanoate would be likely to display similar efficacy. Pharmaceutical formulations including covalently bound corticosteroids and medium-chain fatty acids would also display the advantage of sustained release, for example, as they slowly convert from an ester into their active constituents such as by catalysis with an esterase or carboxyesterase.

Various aspects of this disclosure relate to methods of using pharmaceutical compositions as described herein to treat inflammation, joint pain, joint disease such as osteoarthritis, and related conditions. In some embodiments, the pharmaceutical composition may be administrable locally, topically, or by injection. In some specific embodiments, the pharmaceutical composition may be administrable by intra-articular injection.

The skilled person will immediately recognize many other variations to the pharmaceutical formulations of the present disclosure such as by using a different corticosteroid, a different medium-chain fatty acid or monoglyceride thereof, by adjusting concentrations and excipients, by adding one or more additional active ingredients, and by reformulation of the compositions, for example, for topical use. This summary of the disclosure shall not limit the disclosure or any patent claim that matures therefrom, and any patent claim that grants from this disclosure shall instead be construed according to the plain meaning of the language used in the claim in view of its claim dependency and conventional canons of construction.

### BRIEF DESCRIPTION OF THE DRAWINGS

FIG. 1 is a diagram that depicts the diffusion pathway of dexamethasone (Dex), a corticosteroid, across a cell membrane and into a cell nucleus, supporting embodiments of the present disclosure.

FIG. 2 is a diagram that depicts the lipidomic pathway of phosphatidylinositol 3-kinases (PI3Ks), supporting embodiments of the present disclosure.

FIGS. 3A and 3B are diagrams that depict the movement of prostaglandin E₂ (PGE₂) across a cell membrane, supporting embodiments of the present disclosure.

FIG. 4 is a diagram that depicts sources of arachidonic acid (AA), supporting embodiments of the present disclosure.

FIG. 5 is a bar graph that depicts the concentration of PGE₂ present in the supernatant of normal human chondrocyte cell cultures that were stimulated with interleukin 1-beta (IL-1β) for 48 hours in the presence of varying concentrations of decanoic acid (DA), Dex and lactate (Lac).

FIGS. 6A-6C are parallel-line bioassay graphs depicting shifts in potency for dose-responses of Dex, as measured by reduced IL-1β-induced PGE₂ release, as compared to Dex dose-responses combined with 2.0, 12.5, and 50 micromolar final concentrations of DA (FIGS. 6A, 6B, and 6C respectively).

FIG. 7 is a bar graph that depicts the concentration of monocyte chemoattractant protein 1 (MCP1) in osteoarthritic human synoviocytes cultured in the presence of varying concentrations of DA, Dex, or both DA and Dex.

FIGS. 8A and 8B contain bar graphs that compare the expected and observed effects of the combination of either 1 millimolar DA (FIG. 8A) or 0.5 millimolar DA (FIG. 8B) with varying concentrations of Dex.

FIG. 9A is a bar graph that depicts the concentration of PGE₂ present in the supernatant of osteoarthritic human synoviocyte cell cultures that were stimulated with IL-1β in the presence of varying concentrations of DA and Dex.

FIG. 9B is a scatter plot of the data of FIG. 9A plotted on a logarithmic scale, which defines concentrations required to inhibit 50 percent of the maximum response (IC50) for PGE₂ inhibition.

FIG. 9C is a bar graph that depicts the concentration of prostaglandin D₂ (PGD₂) present in the supernatant of osteoarthritic human synoviocyte cell cultures that were stimulated with IL-1β in the presence of varying concentrations of DA and Dex.

FIG. 10A is a bar graph that depicts IL-1β release from human THP1 macrophages that were stimulated overnight with liposaccharide (LPS) followed by a second, 3-hour proinflammatory stimulus with adenosine triphosphate (ATP) in the presence of various concentrations of DA.

FIG. 10B is a scatter plot of the data in FIG. 10A normalized to the control in each experiment to calculate the percent of IL-1β released in response to various concentrations of DA.

FIG. 10C is a bar graph that depicts IL-1β release from human THP1 macrophages that were stimulated overnight with LPS followed by a second, 3-hour proinflammatory stimulus with ATP in the presence of various concentrations of DA or sodium decanoate.

FIG. 10D is a scatter plot of the data in FIG. 10C normalized to the control in each experiment to calculate the percent of IL-1β released in various concentrations of DA or sodium decanoate.

FIG. 11A is a bar graph that depicts IL-1β release from human THP1 macrophages that were stimulated overnight with LPS followed by a second, 3-hour proinflammatory stimulus with ATP in the presence of various concentrations of dexamethasone phosphate (Dxp).

FIG. 11B is a scatter plot of the data of FIG. 11A normalized to the control in each experiment to calculate the percent of IL-1β released in various concentrations of Dex.

FIG. 12A is a scatter plot that reproduces the data from FIG. 10B (circles) with an overlay for the same experiment performed in the presence of 25 nanomolar Dex (triangles).

FIG. 12B is a scatter plot that reproduces the data from FIG. 11B (circles) with an overlay for the same experiment performed in the presence of 100 micromolar DA (triangles).

FIG. 12C is a scatter plot that reproduces the data from FIG. 10D (grey circles and black triangles) with an overlay for the same experiment where sodium decanoate conditions (black triangles) were performed in the presence of 25 nanomolar Dex (unfilled triangles).

FIG. 13 depicts boxplots divided into panels based on sodium decanoate dilutant: saline or 10 lactated ringers (LR) (25 mM Lac. Each panel depicts IL-1β (picograms per mL) release from human THP1 macrophages that were stimulated overnight with LPS followed by a second, 3-hour proinflammatory stimulus with ATP in the presence of a 5-point concentration curve: 0, 25, 50, 100, 150 micromolar sodium decanoate.

FIG. 14 is a bar graph that displays relative decreases in matrix metalloproteinase 13 (MMP13) expression by osteoarthritic fibroblast-like chondrocytes cultured for 14 or 28 days in the presence of either 0.5 millimolar DA, 0.1 micromolar Dex, or both (DA+Dex).

FIG. 15 is a bar graph that depicts relative expression of messenger RNA (mRNA) levels of key remodeling markers (i.e., Elastin (ELN), matrix metalloproteinase 3 (MMP3), and matrix metalloproteinase 12 (MMP12)) in NHDF treated with sodium Lac followed by activation with transforming growth factor beta-1 (TGF-β1) or IL-1β. To serve as a control, cells were also treated with Dxp.

FIGS. 16 and 17 are bar graphs that present the relative expression of SRY-box transcription factor 9 (SOX9) normalized to glyceraldehyde-3-phosphate dehydrogenase (GAPDH) in high passage, differentiated osteoarthritic chondrocytes stimulated with TGF-β3 and treated with DA, Lac, α-Lipoic acid (LA), and LA+Lac.

FIG. 18 is a bar graph that displays the relative expression of SOX9 versus a control normalized to GAPDH in low passage osteoarthritic chondrocytes treated with Dec, Dxp, Lac, Dec+Dxp, Dec+Lac, Dxp+Lac, and Dec+Dxp+Lac.

FIG. 19 is a bar graph that displays the relative expression of runt-related transcription factor 2 (RUNX2) versus a control normalized to GAPDH in low passage osteoarthritic chondrocytes treated with Dec, Dxp, Lac, Dec+Dxp, Dec+Lac, Dxp+Lac, and Dec+Dxp+Lac.

FIG. 20 is a bar graph that displays the temporal analysis of the relative expression of SOX9 versus RUNX2 in low passage osteoarthritic chondrocytes treated with Dec, Dxp, Lac, Dec+Dxp, Dec+Lac, Dxp+Lac, and Dec+Dxp+Lac.

FIG. 21 is a bar graph that presents the relative expression of MMP13 versus a control normalized to GAPDH in high passage, differentiated osteoarthritic chondrocytes treated with DA, Dec, Dxp, Lac, Dec+Dxp, Dec+Lac, Dxp+Lac, and Dec+Dxp+Lac.

FIG. 22 is a bar graph that depicts the temporal analysis of the relative expression of MMP13 versus a control normalized to GAPDH in low passage osteoarthritic chondrocytes treated with Dec, Dxp, Lac, Dec+Dxp, Dec+Lac, Dxp+Lac, and Dec+Dxp+Lac.

FIG. 23 is a bar graph that displays day 7 relative expression of Aggrecan (ACAN) versus a control normalized to GAPDH in low passage osteoarthritic chondrocytes treated with Dec, Dxp, Lac, Dec+Dxp, Dec+Lac, Dxp+Lac, and Dec+Dxp+Lac.

FIG. 24 is a bar graph that presents day 28 relative expression of type II collagen (Col2a1) versus a control normalized to GAPDH in low passage osteoarthritic chondrocytes treated with Dec, Dxp, Lac, Dec+Dxp, Dec+Lac, and Dxp+Lac.

FIG. 25 is a bar graph that depicts the concentration of prostaglandin I₂ (PGI₂) present in primary human synoviocytes from patients with osteoarthritis that were stimulated with 1 ng/ml final concentration of IL-1β for 24 hours in the presence of varying concentrations of DA and Dex in view of controls.

FIG. 26 is a bar graph that depicts the concentration of 5(S)-Hydroxyeicosatetraenoic Acid (5-HETE) present in primary human synoviocytes from patients with osteoarthritis that were stimulated with 1 ng/mL final concentration of IL-1β for 24 hours in the presence of varying concentrations of DA and Dex in view of controls.

FIG. 27 is a bar graph that depicts the concentration of leukotriene C₄ (LTC₄) present in primary human synoviocytes from patients with osteoarthritis that were stimulated with 1 ng/ml final concentration of IL-1β for 24 hours in the presence of varying concentrations of DA and Dex in view of controls.

FIG. 28 is a bar graph that depicts the concentration of thromboxane B2 (TBX₂) present in primary human synoviocytes from patients with osteoarthritis that were stimulated with 1 ng/ml final concentration of IL-1β for 24 hours in the presence of varying concentrations of DA and Dex in view of controls.

FIG. 29 is a bar graph that depicts an inhibitor screen for the enzyme Cyclooxygenase-2 (COX-2) in the presence of varying concentrations of DA and Dex in view of controls.

FIG. 30 is a bar graph that depicts a Phospholipase A2 (PLA₂) activity assay using PLA₂ isolated from bee venom in the presence of varying concentrations of DA and Dex in view of controls.

FIG. 31A is a bar graph that depicts early concentrations of PGE₂ present in primary human synoviocytes from patients with osteoarthritis that were stimulated with 1 ng/ml final concentration of IL-1β for 24 hours in the presence of varying concentrations of Dex and DA in view of controls.

FIG. 31B is a bar graph depicting PGE₂ release from human osteoarthritic synoviocytes treated with media, 200 nanomolar DA, or 500 nanograms/milliliter of IL-1 Receptor Antagonist (IL-1RA) for the specified pretreatment times at the bottom of each panel (5 minutes, 1 hour, or 2 hours, and then stimulated with 1 nanogram/milliliter IL-1β for either 10 minutes or 1 hour, as indicated.

FIG. 32 is a bar graph that depicts early concentrations of AA present in primary human synoviocytes from patients with osteoarthritis that were stimulated with 1 ng/ml final concentration interleukin 1-beta (IL-1β) for 3 hours in the presence of varying concentrations of Dex and DA in view of controls.

FIG. 33 are bar graphs that depict the concentrations of PGE₂ inside of the cell versus PGE₂ that was secreted into the cell culture media in the presence of varying concentrations of DA and Dex in view of controls.

FIG. 34 is a bar graph that depicts the body weights of rats randomly assigned to seven testing groups over the course of a 28-day MIA-induced Osteoarthritis animal study when treated with various concentrations of saline, LR, DA+LR, Dex+LR, DA+Dex, and DA+Dex+LR.

FIG. 35 is a bar graph that depicts the results of a mechanical sensitivity test (von Frey test) of rats randomly assigned to seven testing groups over the course of a 28-day MIA-induced Osteoarthritis animal study when treated with various concentrations of saline, LR, DA+LR, Dex+LR, DA+Dex, and DA+Dex+LR.

FIG. 36 is a bar graph that depicts the results of a weight bearing test of rats randomly assigned to seven testing groups over the course of a 28-day MIA-induced osteoarthritis animal study when treated with various concentrations of saline, LR, DA+LR, Dex+LR, DA+Dex, and DA+Dex+LR.

FIG. 37 is a bar graph that depicts the concentration of potent pro-inflammatory cytokine, Tumor Necrosis Factor-Alpha (TNF-α), of rats randomly assigned to seven testing groups when treated with various concentrations of saline, LR, DA+LR, Dex+LR, DA+Dex, and DA+Dex+LR.

FIG. 38 is a bar graph that depicts the concentration of potent pro-inflammatory cytokine, IL-1β, of rats randomly assigned to seven testing groups when treated with various concentrations of saline, LR, DA+LR, Dex+LR, DA+Dex, and DA+Dex+LR.

FIG. 39 is a bar graph that depicts the concentration of potent pro-inflammatory cytokine, interleukin-6 (IL-6), of rats randomly assigned to seven testing groups when treated with various concentrations of saline, LR, DA+LR, Dex+LR, DA+Dex, and DA+Dex+LR.

FIG. 40 is a bar graph that depicts the concentration of a marker of cartilage degradation, MMP3, of rats randomly assigned to seven testing groups when treated with various concentrations of saline, LR, DA+LR, Dex+LR, DA+Dex, and DA+Dex+LR.

FIG. 41 is a bar graph that depicts the concentration of a marker of cartilage degradation, MMP13, of rats randomly assigned to seven testing groups when treated with various concentrations of saline, LR, DA+LR, Dex+LR, DA+Dex, and DA+Dex+LR.

FIG. 42 is a bar graph that depicts the concentration of a marker of cartilage degradation, C-terminal telopeptides of type II collagen (CTX-II), of rats randomly assigned to seven testing groups when treated with various concentrations of saline, LR, DA+LR, Dex+LR, DA+Dex, and DA+Dex+LR.

FIG. 43 is a bar graph that depicts the concentration of a marker of cartilage degradation, cartilage oligomeric matrix protein (COMP), of rats randomly assigned to seven testing groups when treated with various concentrations of saline, LR, DA+LR, Dex+LR, DA+Dex, and DA+Dex+LR.

FIGS. 44A and 44B are bar graphs that depict the articular cartilage surface area and surface area change of rats randomly assigned to seven testing groups when treated with various concentrations of saline, LR, DA+LR, Dex+LR, DA+Dex, and DA+Dex+LR.

FIGS. 45A and 45B are bar graphs that depict the articular cartilage volume and volume change of rats randomly assigned to seven testing groups when treated with various concentrations of saline, LR, DA+LR, Dex+LR, DA+Dex, and DA+Dex+LR.

FIGS. 46A and 46B are bar graphs that depict the articular cartilage thickness and thickness change of rats randomly assigned to seven testing groups when treated with various concentrations of saline, LR, DA+LR, Dex+LR, DA+Dex, and DA+Dex+LR.

FIG. 47 is a bar graph that depicts the mankin scores of rats randomly assigned to seven testing groups when treated with various concentrations of saline, LR, DA+LR, Dex+LR, DA+Dex, and DA+Dex+LR.

FIG. 48 is a bar graph that depicts the synovial injury scores of rats randomly assigned to seven testing groups when treated with various concentrations of saline, LR, DA+LR, Dex+LR, DA+Dex, and DA+Dex+LR.

FIG. 49 is a bar graph that depicts relative expression of RUNX2 and SOX9 in low passage human osteoarthritic chondrocytes treated with 100 μM decanoate (Dec), 0.1 μM Dxp, 100 mM d/l Lac, and combinations (n=4; * p-value=>0.05 one sample, two-tailed t-test versus hypothetical 1).

FIG. 50 is a bar graph that depicts relative expression of SOX9 normalized to RunX2 in low passage human osteoarthritic chondrocytes treated with 100 μM Dec, 0.1 μM Dxp, 100 mM d/l Lac, and combinations (n=4; * p-value=>0.05 one sample, two-tailed t-test versus hypothetical 1).

FIG. 51 is a bar graph that depicts relative expression of type II collagen (Col2a1) in low passage human osteoarthritic chondrocytes treated with 100 μM Dec, 0.1 μM Dxp, 100 mM d/l Lac, and combinations (n=3).

FIG. 52 is bar graph that depicts relative expression of type I collagen (Col1a1) in low passage human osteoarthritic chondrocytes treated with 100 μM Dec, 0.1 μM Dxp, 100 mM d/l Lac, and combinations (n=3).

FIG. 53 is a bar graph that depicts relative expression of ACAN in low passage human osteoarthritichondrocytes treated with 100 μM Dec, 0.1 μM Dxp, 100 mM d/l Lac, and combinations (n=3).

FIG. 54 is a bar graph that depicts a 24-hour model comparison of PGE₂ release evaluated for passage 3 human chondrocytes comparing pre-treatment for 24 hours, administering at the same time as activation, or by adding to cells following an overnight activation with IL-1β when treated with various concentrations of Dec, Dxp, LA, and Lac.

FIG. 55 is a bar graph that depicts a 96-hour model comparison of PGE₂ release evaluated for passage 3 human chondrocytes comparing pre-treatment for 24 hours, administering at the same time as activation, or by adding to cells following an overnight activation with IL-1β when treated with various concentrations of Dec, Dxp, LA, and Lac.

FIG. 56 is a bar graph that depicts the fold increases from the top 9 significant lipids that were modulated by DA in Example 21.

FIG. 57 is a chart that depicts molecular pathway interactions with DA lipidomics, specifically DA's effect on PGE₂ efflux.

### DETAILED DESCRIPTION

Embodiments of pharmaceutical compositions described herein generally include varying concentrations of a lipid selected from medium-chain fatty acids and carboxylates and monoglycerides thereof, and a corticosteroid, alone or in combination with other chemicals and additives (e.g., tryptophan (Try), hyaluronic acid (HA), lactate (Lac), water).

As described herein, lipids generally display anti-inflammatory properties that are particularly relevant to osteoarthritis and the combination of a corticosteroid and a lipid advantageously enable osteoarthritis to be treated with less corticosteroid. As discussed above, repeated injections of corticosteroids at high doses, for example, can cause severe side effects such as cartilage loss and bone fractures. The inclusion of a lipid enables the treatment of osteoarthritis with a subclinical dose of the corticosteroid that reduces the risk of side effects caused by the corticosteroid relative to a clinical dose. Additionally, the subclinical dose of the corticosteroid is also a therapeutically-effective amount of the corticosteroid that has a clinically-relevant effect at treating osteoarthritis. Thus, the combination of the lipid with the corticosteroid enables treatment with a therapeutically-effective amount of the corticosteroid while reducing the risk of side effects caused by the corticosteroid relative to a clinical dose.

In some embodiments, the corticosteroid includes dexamethasone (Dex) or ester thereof and the lipid is selected from medium-chain fatty acids and carboxylates and monoglycerides thereof. In some embodiments, the lipid is polysaturated. In some specific embodiments, the lipid is decanoic acid (DA) (capric acid). This disclosure teaches that lipids in general, and specifically DA, have anti-inflammatory properties that are particularly relevant to osteoarthritis and advantageously display synergy when combined with Dex.

Additional embodiments of this disclosure relate to pharmaceutical compositions comprising a corticosteroid, such as Dex or ester thereof, a lipid selected from medium-chain fatty acids and carboxylates and monoglycerides thereof, and Lac, such as Ringer's lactate (LR). As described herein, Lac also has a beneficial effect on inflammation, collagen production, and the re-differentiation of chondrocytes. Thus, some embodiment described herein include an aqueous injectable solution that may include Dex phosphate (e.g., Dex-21-phosphate) (Dxp), LR, and DA, for example, that reduces the overall amount of Dxp to be administered to the patient to reduce the risk of side effects that confound the treatment of osteoarthritis and other conditions by corticosteroid injection.

Specifically, as described above, chronic inflammation and/or osteoarthritis can be treated, for example, with NSAIDS, tumor necrosis factor (TNF) inhibitors, and corticosteroids. NSAIDs generally inhibit the activity of cyclooxygenase enzymes while TNF inhibitors typically bind TNF, preventing ligand-receptor binding, which suppresses two important inflammatory signaling cascades. Singularly targeting specific pathological signaling pathways, however, may not reverse harm from chronic or overactive inflammation alone. The identification of novel strategies to treat overall inflammation or upstream modalities is desirable, and strategies that prevent or reverse underlying pathologies could foster a paradigm shift in how physicians manage inflammation. For example, as discussed herein, the novel combination of a corticosteroid and a lipid to treat osteoarthritis is shown to reduce inflammation.

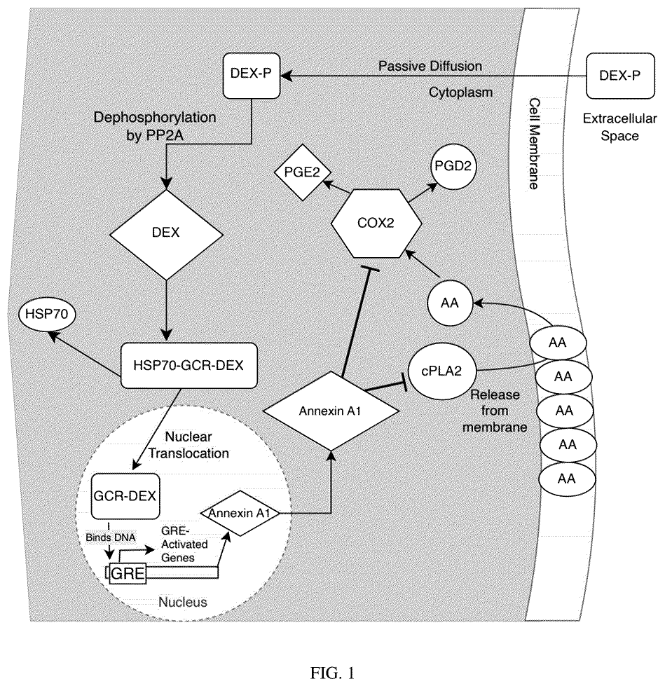

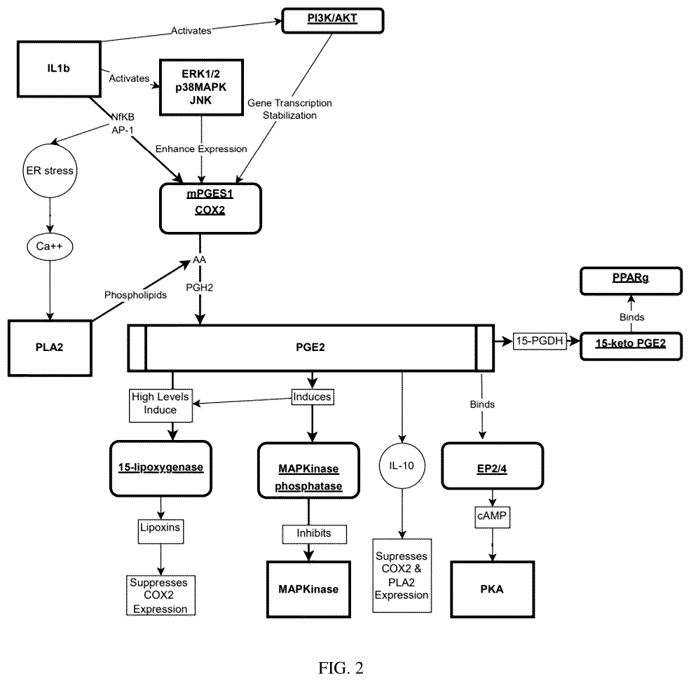

Corticosteroids, due to their lipophilic nature, passively enter the cytoplasm and bind to intracellular steroid receptors. FIG. 1 depicts the diffusion pathway of Dex across a cell membrane and into a cell nucleus. These interactions can result in the active transport of the ligand/receptor complex into the nucleus, where corticosteroids exert their effects through gene expression or the regulation of concurrent signaling cascades. Dex, for example, inhibits inflammatory signaling that upregulates cyclooxygenase-2, an enzyme in the prostaglandin synthesis pathway. FIG. 2 depicts the lipidomic pathway of PI3Ks, supporting embodiments of the present disclosure, Corticosteroids inhibit many inflammatory pathways; thus, strategies that enhance or complement the activity of corticosteroids may provide novel ways to inhibit inflammation from progressing.

An intriguing target for treating chronic inflammatory conditions such as osteoarthritis is a cytosolic, multiprotein complex known as the inflammasome. Inflammasomes are multi-protein complexes that assemble in the cytosol of cells in response to various innate immunological stimuli (for example, pathogen-associated molecular pattern molecules (PAMPs) and damage-associated molecular pattern molecules (DAMPs)). These pinwheel-like structures serve as a scaffold to dimerize and activate inflammatory proteases known as caspases. The inflammasome assembles during inflammation to promote caspase-1-mediated cleavage of the proinflammatory cytokines IL-1β and interleukin 18 (IL-1β). Together with other cellular insults (for example, ATP release and potassium efflux), inflammasomes drive the maturation of IL-1β and gasdermin D. IL-1β concentration therefore directly correlates with inflammasome activity, and compounds that decrease IL-1β therefore typically decrease inflammasome activity.

Moreover, inflammasome activation generally initiates other inflammatory signaling cascades. Thus, inflammasome inhibitors could potentially quiesce several different inflammatory pathways and even prevent the progression of inflammation. The identification of an inflammasome antagonist that displays efficacy at inhibiting multiple inflammatory signaling pathways nevertheless remains elusive. Such an antagonist would likely provide therapeutic benefits in the treatment of chronic inflammatory diseases including osteoarthritis.

Another intriguing target for treating chronic inflammatory conditions such as osteoarthritis is regulatory transcription factors associated with the development and differentiation of chondrocytes. Chondrocytes are essential for the overall health of articular joints because they help maintain an equilibrium between anabolic and catabolic substances produced in the microenvironment. Two master regulatory transcription factors that aid in this process are SRY-box transcription factor 9 (SOX9) and runt-related transcription factor 2 (RUNX2). SOX9 secures proper chondrocyte lineage commitment, promotes cell survival, and regulates cartilage-specific structural components such as type II collagen (Col2a1) and Aggrecan (ACAN). Conversely, RUNX2 drives the expression genes for cartilage degradation, hypertrophic differentiation, and ossification, such as matrix metalloproteinase 13 (MMP13). Furthermore, overexpression of SOX9 has been shown to ameliorate the course of experimental osteoarthritis, whereas RUNX2 overexpression causes post-traumatic osteoarthritis progression. Further, SOX9 and RUNX2 levels are essential for converting chondrocytes into osteoblasts. Osteoblasts are bone-forming, and their dysregulation has been associated with the development of osteoarthritis. According to studies, SOX9 may prevent chondrocytes from dedifferentiating into skeletogenic precursor cells that can then transdifferentiate into osteoblasts. In contrast, RUNX2 enhances the survival of hypertrophic chondrocytes, which can differentiate into osteoblasts. Furthermore, Col1a1 is regarded as both a marker of osteoblast lineage and fibrosis. Embodiments of the present disclosure are directed to the regulation of SOX9 and RUNX2 and prevention of osteoblast development.

Further, a hallmark of osteoarthritis is an early and profound loss of ACAN. ACAN is a proteoglycan decorated with glycosaminoglycan side chains that binds to hyaluronan and other proteins to form hydrated gel structures, allowing cartilage to endure compressive pressures. Proteolytic enzymes specific to ACAN are found in arthritic cartilage and are responsible for its breakdown, which is thought to contribute to pathogenesis. Embodiments of the present disclosure are directed to inhibiting ACAN degradation and/or restore its production.

Embodiments of the pharmaceutical compositions described herein address the severe side effects associated with the use of a corticosteroid alone for the treatment of osteoarthritis because the addition of a lipid (1) modulates human monocytes and/or macrophages to reduce IL-1β signaling and (2) independently modulates human osteoarthritic chondrocytes to promote their re-differentiation. Specifically, the lipid is therapeutically effective to treat inflammation and/or osteoarthritis. Thus, compositions comprising the corticosteroid and a lipid according to this disclosure can advantageously reduce inflammasome-mediated inflammation, positively impact chondrocyte development and differentiation, advantageously regulate osteoblast conversion, advantageously inhibit ACAN degradation and/or ACAN restoration, reduce oxidative stress, and replace degraded synovial fluid, among others. More specifically, at least some embodiments relate to pharmaceutical compositions comprising a corticosteroid and a lipid. In some embodiments, the corticosteroid and the lipid are present in the pharmaceutical composition at amounts that are therapeutically effective to treat inflammation, osteoarthritis, or both. Compositions comprising a lipid and corticosteroid according to this disclosure can advantageously reduce inflammasome-mediated inflammation. Without limiting this disclosure or any patent claim that matures from this disclosure, medium-chain fatty acids can bind to and inhibit intracellular inflammasomes, which can reduce IL-1β signaling and corresponding inflammation.

As discussed herein, the addition of the lipid to the corticosteroid reduces IL-1β signaling and corresponding inflammation. IL-1β promotes synovitis, cartilage loss, osteophyte formation, and the dedifferentiation of chondrocytes. Macrophages are a primary source of IL-1β. Some aspects of this disclosure relate to compositions that inhibit the release of IL-1β by macrophages and that also increase collagen production (for example, as measured by Col2a1 expression). Such compositions can advantageously treat osteoarthritis by reducing inflammation and disease progression, by restoring lost collagen, and by promoting cartilage repair by re-differentiating osteoarthritic chondrocytes into healthy chondrocytes.

Pain associated with inflammation can be treated directly with analgesics such as opiates, which generally target opioid receptors in the brain. Additionally, N-methyl-D-aspartate (NMDA) receptor antagonists are known to display analgesic effects, which antagonists include, for example, ketamine and nitrous oxide. However, NMDA receptor antagonists are not generally prescribed for long-term pain management or to treat pain associated with inflammation.

The NMDA receptor is a glutamate receptor and calcium ion channel. The binding of two glutamates to an NMDA receptor activates the calcium channel to increase calcium permeability. Antagonists such as ketamine and nitrous oxide block the calcium channel. NMDA receptor activation can contribute to the development and maintenance of chronic pain conditions by inducing sensitization of pain-sensing neurons, and the NMDA receptor therefore plays a role in synaptic plasticity and pain.

Glutamate, which functions as a neurotransmitter when binding NMDA receptors, is also an amino acid building block of proteins and a precursor and metabolite in numerous other biochemical pathways. Glutamate is notably the transamination product of the citric acid cycle intermediate alpha-ketoglutarate (AKG), and a number of different enzymes interconvert glutamate and AKG.

The citric acid cycle (which is also known as the Krebs cycle) is a series of enzymatic reactions that take place in the mitochondria and generates energy in the form of adenosine triphosphate (ATP). Beta-oxidation of fatty acids breaks down fatty acids to produce acetyl-CoA, which enters the citric acid cycle in the mitochondria to generate ATP. AKG is not directly formed from fatty acids. Fatty acids first undergo beta-oxidation to generate acetyl-CoA, which can be converted to AKG. The enzyme glutamate dehydrogenase can catalyze the conversion of glutamate into AKG and NADPH, which NADPH is involved in cellular processes such as fatty acid synthesis and antioxidant defense. Whether pharmacological manipulation of the citric acid cycle or its intermediates can affect NMDA receptor activation to produce therapeutic effects, for example, by modulating glutamate concentration, remains unknown. Without limiting this disclosure or any patent claim that matures from this disclosure, lipids such as medium-chain fatty acids can drive the citric acid cycle to increase AKG production and modulate its homeostatic equilibrium with glutamate thereby affecting NMDA activation and sensitization to pain.

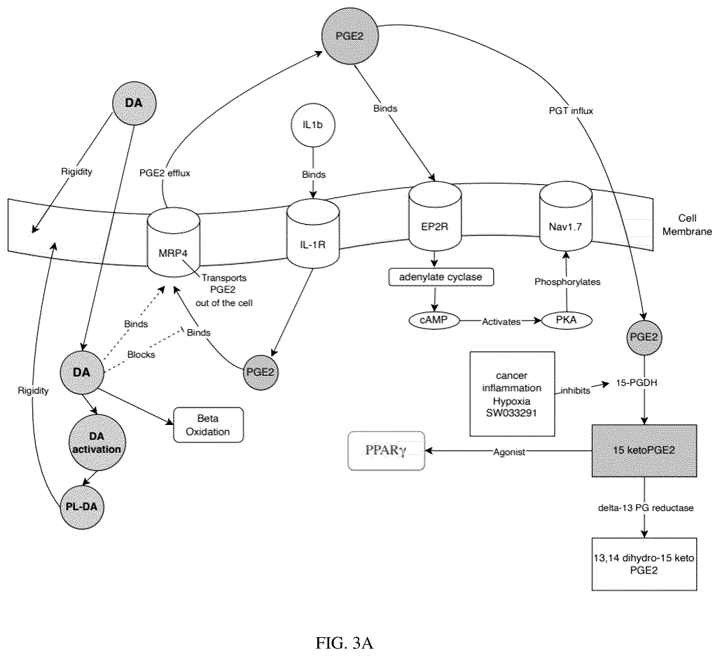

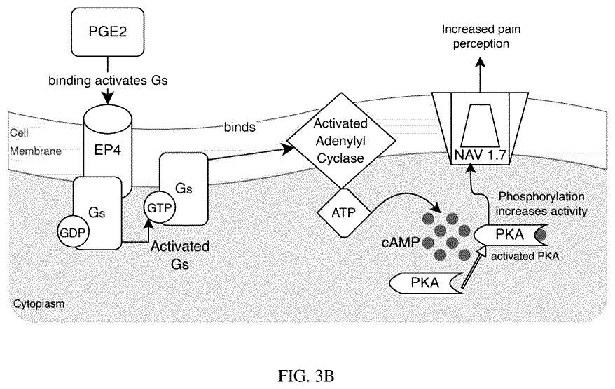

Additionally, chondrocytes express both the NMDA calcium channel and the voltage-gated sodium channel Naᵥ1.7, which regulates intracellular calcium signaling. Osteoarthritis correlates with Naᵥ1.7 expression, and pharmacological blockade of Naᵥ1.7 both inhibits the progression of osteoarthritis and appears to inhibit osteoarthritis-associated pain. IL-1β induces increased Naᵥ1.7 expression in chondrocytes. FIGS. 3A and 3B depict the movement of prostaglandin E₂ (PGE₂) across a cell membrane, supporting embodiments of the present disclosure. The corticosteroid and lipid formulations described herein synergistically reduce IL-1β release as well as downstream inflammatory response including the biosynthesis of PGE₂, and they ultimately redifferentiate chondrocytes from fibroblast-like phenotypes into normal chondrocytes. PGE₂ is a potent inflammatory mediator that is part of the eicosanoid family of arachidonic acid (AA)-derived molecules. Studies demonstrate that it plays a significant role in Osteoarthritis inflammation and pain and elevated levels have been found in osteoarthritis and are associated with the loss of subchondral bone and articular cartilage. Without limiting this disclosure or any patent claim that matures from this disclosure, the formulations described herein redifferentiate chondrocytes in part by downregulating Naᵥ1.7 expression and/or activity by synergistically inhibiting IL-1β-mediated signaling pathways that would otherwise result in the increased expression of Naᵥ1.7 that perpetuates the osteoarthritic phenotype. In other words, co-formulations comprising a corticosteroid and a lipid can ameliorate the disease phenotype of osteoarthritic chondrocytes by reducing their expression of Naᵥ1.7.

Additionally, recent advancements in chondrocyte biology suggest that voltage-gated sodium channels may be targeted for therapeutic intervention and disease modification of OA. Voltage-gated sodium channels have been traditionally associated with excitable cells, such as neurons and muscles, but studies have identified these channels in OA-associated chondrocytes. Notably, pharmacological inhibition of Naᵥ1.7 has been shown to alleviate both pain and structural damage in OA. The proposed mechanisms suggest that Ca2+-induced release of disease mediators from chondrocytes plays a key role. Additionally, emerging evidence points to the involvement of these channels in the migration, invasion, and cytokine release of innate immune cells. Accordingly, embodiments of the pharmaceutical compositions described herein may inhibit Naᵥ1.7 and/or induce the release of disease mediators from chondrocytes using Ca2+ to treat OA.

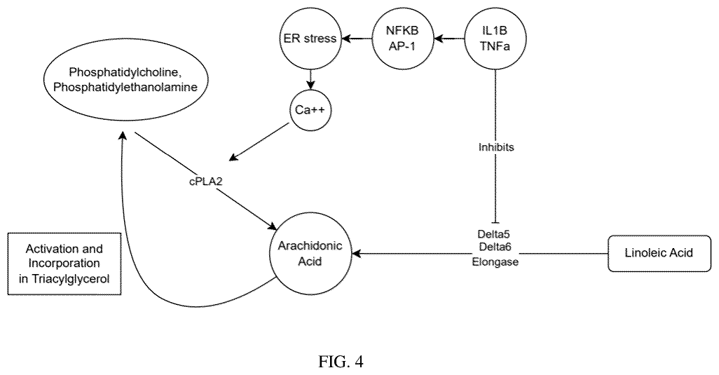

The fatty acid AA also plays a critical role in inflammation. FIG. 4 depicts sources of AA, supporting embodiments of the present disclosure, Phospholipid enzymes, such as phospholipase C and PLA₂, convert diacylglycerol and phospholipids, respectively into AA. Cyclooxygenases (e.g., prostaglandin H₂ (PGH₂) synthase, Cyclooxygenase-1 (COX-1) or Cyclooxygenase-2 (COX-2) and peroxidase) convert AA into prostaglandins such as PGE₂ and PGH₂. Further, PGH₂ may be converted into PGI₂ and thromboxane (TXA₂) via prostacyclin synthase and thromboxane synthase, respectively. AA may also be converted to leukotrienes, biological mediators that contribute to inflammation. LTC₄ is produced when glutathione is conjugated with leukotriene A₄ (LTA₄), a leukotriene that is a product of lipoxygenase acting on AA to produce hydroperoxyeicosatetraenoic acid (HPETE, 5-HETE). Without limiting this disclosure or any patent claim that matures from this disclosure, as least some of the lipids described herein may specifically bind the enzyme active site that AA binds as the enzyme catalyzes its conversion into PGE₂ or another prostaglandin. In the enzyme active site, AA folds back upon itself to enable the formation of an intramolecular bond to produce a carbon homocycle. DA contains half as many carbons as AA and PGE₂, and thus, either a single DA molecule fits into half of the AA binding site or two DA molecules fill the entire binding site. In either scenario, the medium-chain fatty acid acts as a competitive inhibitor of the cyclooxygenase.

Unlike long-chain fatty acids that are much more prevalent in humans, medium-chain fatty acids possess appreciable solubility in water at concentrations up to about 1 millimolar. The inventors discovered that medium-chain fatty acids can therefore be administered at pharmaceutically-relevant concentrations in aqueous formats that allow the medium-chain fatty acids to partition from the extracellular fluid into cells whereas long-chain fatty acids are insoluble at pharmaceutically-relevant concentrations. Prostaglandin D₂ (PGD₂) synthase converts AA into PGD₂. Medium-chain fatty acids as well as carboxylates and monoglycerides thereof would be expected to display effects on both PGD₂ synthase and PGD₂ similar to the effects on cyclooxygenases and PGE₂. Free fatty acids and carboxylates and monoglycerides thereof may also inhibit or reverse the activity of phospholipases generally and PLA2 specifically. PLA2 hydrolyzes phosphatidylcholine into 1-acylglycerophosphaocholine and a carboxylate of a free fatty acid. Certain families of PLA2 release the carboxylate of AA thereby increasing the concentration of AA available for conversion by cyclooxygenases into prostaglandins (such as PGE₂) and driving pro-inflammatory pathways. Without limiting this disclosure or any patent claim that matures from this disclosure, increased concentrations of free fatty acids can either drive PLA2 catalysis in the opposite direction favoring the production of phosphatidylcholine rather than the production of AA or otherwise block the binding site for phosphatidyl choline. Carboxylates and monoglycerides of fatty acids would display a similar effect.

Without limiting this disclosure or any patent claim that matures from this disclosure, medium-chain fatty acids bind to the LPS-binding sites on inflammasome caspase activation and recruitment domains (CARDs), which inhibits inflammasomes. Additionally, without limiting this disclosure or any patent claim that matures from this disclosure, medium-chain fatty acids can also serve as a carbon source for the citric acid cycle, which can increase concentrations of the citric acid cycle intermediate AKG, which AKG improves nitrogen transport and displays antioxidant properties. Moreover, without limiting this disclosure or any patent claim that matures from this disclosure, medium-chain fatty acids also bind a specific locus on the NACHT domain on the NLRP3 inflammasome that possesses ATPase activity to inhibit activation of the NLRP3 inflammasome. Inhibition of the NACHT domain locus, for example, with the small molecule MCC950 is known to inhibit activation of the NLRP3 inflammasome. Finally, without limiting this disclosure or any patent claim that matures from this disclosure, medium-chain fatty acids can also inhibit the NLRP3 inflammasome by binding its NACHT domain.

Regardless of their precise mechanism of action, the examples provided below suggest that medium-chain fatty acids in general, and specifically DA, (1) modulate human monocytes and/or macrophages to reduce IL-1β signaling, and (2) independently modulate human osteoarthritic chondrocytes to promote their re-differentiation. Each of these effects has an independent, favorable impact on osteoarthritis. Similar medium-chain free fatty acids are expected to display similar effects as well as their monoglyceride counterparts.

In some embodiments, the pharmaceutical composition is effective to reduce inflammation, reduce progression of osteoarthritis, and/or re-differentiate osteoarthritic chondrocytes. In some specific embodiments, the components of the pharmaceutical composition, such as a lipid and the corticosteroid, are synergistically effective to reduce inflammasome-mediated inflammation. In some specific embodiments, the lipid and the corticosteroid are synergistically effective to reduce IL-1β.

In some embodiments, a steroid of the pharmaceutical composition is a corticosteroid. In some embodiments, the corticosteroid is Dex or an ester thereof. In some specific embodiments, the ester is selected from Dxp, Dex sulfate, Dex acetate, Dex propionate, Dex valerate, Dex pivalate, Dex tert-butylacetate, Dex succinate, Dex troxundate, Dex 17-propionate, Dex dipropionate, Dex metasulphobenzoate, Dex isonicotinate, Dex diethylaminoacetate, Dex acefurate, Dex cipecilate, Dex octanoate, Dex decanoate, Dex palmitate, and Dex linoleate. In some very specific embodiments, the corticosteroid is Dex, Dxp, Dex sulfate, or Dex acetate. In alternative embodiments, the corticosteroid may be any other corticosteroid. For example, in some embodiments, the corticosteroid is selected from cortisol, cortisone, triamcinolone, prednisone, prednisolone, methylprednisolone, and betamethasone.

In some embodiments, the pharmaceutical composition comprises the corticosteroid at a concentration of at least 400 picomolar and up to 20 millimolar. In some specific embodiments, the pharmaceutical composition comprises the corticosteroid at a concentration of at least 800 picomolar and up to 800 nanomolar. In some very specific embodiments, the pharmaceutical composition comprises the corticosteroid at a concentration of at least 1.2 nanomolar and up to 100 nanomolar.

In some embodiments, the lipid is a medium-chain fatty acid such as a polysaturated medium-chain fatty acid. Exemplary medium-chain fatty acids include hexanoic acid (caproic acid), octanoic acid (caprylic acid), DA (capric acid), and dodecanoic acid (lauric acid). In some specific embodiments, the lipid is DA.

In some embodiments, the lipid is a carboxylate of a medium-chain fatty acid, which interconvert with molecular medium-chain fatty acids, for example, when dissolved in aqueous solution. In some specific embodiments, the lipid is a carboxylate selected from hexanoate (caproate), octanoate (caprylate), decanoate (caprate), and dodecanoate (laurate). In some very specific embodiments, the lipid is a carboxylate, and the carboxylate is dissolved in a solvent such as water. In some very specific embodiments, the lipid is a carboxylate, and the carboxylate is present as a salt such as a sodium, potassium, calcium, or magnesium salt.

In some embodiments, the lipid is a monoglyceride of a medium-chain fatty acid. At least a portion of free fatty acids are converted into monoglycerides in vivo. Without limiting this disclosure or any patent claim that matures from this disclosure, monoglycerides display activity against inflammation in osteoarthritis. Free fatty acids are converted into monoglycerides in vivo first by acetyl-CoA synthetase, which converts a free fatty acid, coenzyme A (CoA), and ATP into adenosine monophosphate (AMP), pyrophosphate, and a thioester of the free fatty acid and CoA. Monoglyceride acyltransferase then converts the thioester and glycerol into a monoglyceride and CoA. Exemplary monoglycerides of medium chain fatty acids include monohexanoin (monocaproin), monooctanoin (monocaprylin), monodecanoin (monocaprin), and monododecanoin (monolaurin). In some specific embodiments, the monoglyceride is monodecanoin.

In some embodiments, the pharmaceutical composition comprises the lipid at a concentration of at least 10 micromolar and up to 5 millimolar. In some specific embodiments, the pharmaceutical composition comprises the lipid at a concentration of at least 10 micromolar and up to 1 millimolar. In some very specific embodiments, the pharmaceutical composition comprises the lipid at a concentration of at least 25 micromolar and up to 500 micromolar. Without limiting this disclosure or any patent claim that matures from this disclosure, concentrations of either medium chain fatty acids or monoglycerides thereof are believed to be toxic at concentrations greater than 5 millimolar (see, for example, U.S. patent application Ser. No. 18/424,626, filed Jan. 26, 2024, and its corresponding publication, which is incorporated by reference in its entirety).

In some embodiments, the medium-chain fatty acid has a conjugate base, which is the carboxylate. In some embodiments, the composition comprises a carboxylate, wherein the carboxylate is the conjugate base of the medium-chain fatty acid. In some embodiments, the medium-chain fatty acid comprises a conjugate base, which is a carboxylate, and the composition comprises the carboxylate. DA, for example, has a pKa (negative log of its acid dissociation constant) of about 4.9, which means that aqueous phases that comprise DA generally also include its conjugate base decanoate, at least at neutral pH. Various compositions of this disclosure comprise water, and a portion of DA that dissolves in the water will be deprotonated to form dissolved decanoate. Decanoate is a sodium salt of DA with superior aqueous solubility characteristics. The solubility of DA in water is about 150 parts per million by mass. Aqueous compositions of this disclosure may nevertheless include a cosolvent and/or surfactant, for example, which may increase the concentration of a dissolved medium-chain fatty acid above its nominal solubility in water.

In some embodiments, the composition comprises a combined concentration of the medium-chain fatty acid and the carboxylate of at least 300 parts per million and up to 3 percent by mass. In some specific embodiments, the composition comprises a combined concentration of the medium-chain fatty acid and the carboxylate of at least 1,000 parts per million and up to 1.5 percent by mass. In some even more specific embodiments, the composition comprises a combined concentration of the medium-chain fatty acid and the carboxylate of at least 1,133 parts per million and up to 1.02 percent by mass. In some very specific embodiments, the composition comprises a combined concentration of the medium-chain fatty acid and the carboxylate of at least 1,700 parts per million and up to 6,800 parts per million by mass. Lower combined concentrations of a medium-chain fatty acid and carboxylate display lower efficacy, and higher combined concentrations risk toxicity.

In some embodiments, the composition comprises water. In some specific embodiments, a majority of the corticosteroid is dissolved in the water. In some very specific embodiments, a majority of the corticosteroid is dissolved in the water, and a majority of the lipid is dissolved in the water. Dxp, for example, has a net negative charge, which increases its solubility in water relative to neutrally-charged Dex. In some embodiments, the pharmaceutical composition is an aqueous composition that comprises a dissolved corticosteroid solute at a concentration of at least 1 milligram per milliliter and up to 10 milligrams per milliliter.

Aspects of the present disclosure are also directed to the use of lipid mediators in the treatment of osteoarthritis. Maresin 2 (MaR2, 13R,14S-dihydroxy-docosahexaenoic acid) is a specialized pro-resolving lipid mediator (SPM) derived from docosahexaenoic acid (DHA, 22:6, n-3), and plays a crucial role in inflammation resolution, tissue repair, and immune modulation, distinguishing itself from traditional anti-inflammatory drugs that only suppress inflammation without actively promoting resolution. MaR2 represents a powerful endogenous pro-resolving mediator that offers a novel approach to controlling inflammation, promoting tissue healing, and reducing pain. Embodiments of the present disclosure are directed to modulating cytokine production, enhance macrophage function, regulate ECM remodeling, and alter pain pathways via therapeutics aimed at inflammation resolution and tissue regeneration.

In some embodiments, the pharmaceutical composition comprises one or more polysaccharides, such as HA. HA is a naturally occurring polysaccharide, found in connective tissues and synovial fluid, that serves to lubricate and cushion the joint. In osteoarthritis, HA injections are used as a viscosupplement to help replace degraded synovial fluid; thus, reducing pain and improving joint function. However, its effectiveness varies, with some patients experiencing significant relief while others see minimal benefit. Moreover, its effects can be short-lived, requiring repeated injections, and in rare situations, inflammatory reactions may occur. Embodiments of the present disclosure are directed to promoting the beneficial effects of HA in treating osteoarthritis. In some embodiments, the composition comprises HA. In some embodiments, the compositions comprise HA as a solution and as a HA hydrogel. Additionally, in some embodiments, the composition comprises a hydrogel, wherein the hydrogel comprises HA and water, and the HA has a molecular weight of at least 500 kilodaltons. In some specific embodiments, the HA is high molecular weight HA, which has a molecular weight of at least 1000 kilodaltons. In some very specific embodiments, the HA is high molecular weight HA, which has a molecular weight of at least 5000 kilodaltons. SYNVISC® (Sanofi-Aventis, United States), for example, comprises HA that has an average molecular weight of about 6000 kilodaltons. The composition may comprise therapeutically effective amounts of HA. In some embodiments, the pharmaceutical composition comprises HA at a concentration of at least 0.01 percent and up to 1 percent.

In some embodiments, the pharmaceutical composition comprises one or more amino acids, such as Try. Fatty acid and Try metabolism play key roles in chondrocyte function and osteoarthritis progression, particularly in inflammation and cartilage homeostasis. Fatty acids influence chondrocyte energy production and inflammatory responses, with an imbalance in metabolism promoting inflammation and cartilage degradation. Moreover, Try metabolism generates bioactive metabolites, including kynurenine, which modulate immune responses and inflammation in the joint environment. Dysregulation of these pathways can contribute to osteoarthritis by promoting chronic inflammation and oxidative stress, accelerating cartilage breakdown. Targeting these metabolic pathways could offer new therapeutic approaches for osteoarthritis management. Embodiments of the present disclosure are directed to stabilizing chondrocyte metabolism and producing biological metabolites that promote chondrogenesis. As such, in some embodiments, Try may be included in the composition as a free amino acid, which generally exists as a mixture of zwitterionic and neutral tautomers in compositions of that comprise an aqueous phase. In this disclosure, the term “tryptophan” and “Try” includes both zwitterionic and neutral tautomers of Try and both enantiomers of Try.

In some embodiments, the pharmaceutical composition comprises one or both of L-Try and D-Try. In some specific embodiments, the composition comprises a racemic mixture of L- and D-Try. In some specific embodiments, the composition comprises L-Try, and the composition lacks D-Try.

In some embodiments, the composition comprises Try at a concentration of at least 2,700 parts per million and up to 10 percent by mass. In some specific embodiments, the composition comprises Try at a concentration of at least 6,000 parts per million and up to 8 percent by mass. In some even more specific embodiments, the composition comprises Try at a concentration of at least 9,000 parts per million and up to 8.1 percent by mass. In some very specific embodiments, the composition comprises Try at a concentration of at least 1.3 percent and up to 5.4 percent by mass.

In some embodiments, the pharmaceutical composition comprises one or more antioxidants, such as α-lipoic acid (LA). In some embodiments, the composition comprises LA at a concentration of at least 0.5 micromolar, or more particularly of at least 1 micromolar, or more particularly of at least 2 micromolar, or more particularly of at least 3 micromolar, or more particularly of at least 4 micromolar, or more particularly of at least 4 micromolar, or more particularly of at least 5 micromolar, or more particularly of at least 6 micromolar, or more particularly of at least 7 micromolar, or more particularly of at least 8 micromolar, or more particularly of at least 9 micromolar, or more particularly of at least 10 micromolar.

In some embodiments, the composition also comprises Lac. As described in the examples below, Lac treatment of chondrocytes promotes metabolic management, which is beneficial during inflammatory stages of osteoarthritis and promotes transcriptional programs that preserve cartilage. Accordingly, the addition of Lac has a beneficial effect on inflammation, collagen production, and the re-differentiation of chondrocytes. Thus, some embodiment described herein include an aqueous injectable solution that may include Dxp, LR, and DA, for example, that reduces the overall amount of Dxp to be administered to the patient to reduce the risk of side effects that confound the treatment of osteoarthritis and other conditions by corticosteroid injection.

In some specific embodiments, the composition comprises at least 2 millimolar and up to 500 millimolar Lac. In some very specific embodiments, the composition comprises at least 20 millimolar and up to 50 millimolar Lac.

In some embodiments, the composition is a modified LR solution that has been modified to comprise the lipid and the corticosteroid.

Various aspects of this disclosure relate to a method to treat inflammation, comprising providing a pharmaceutical composition as described anywhere herein and administering the pharmaceutical composition to a patient.

Various aspects of this disclosure relate to a method to treat inflammation, the method comprising providing a pharmaceutical composition as described anywhere herein and administering the pharmaceutical composition to an osteoarthritic joint of a patient. An osteoarthritic joint is a joint, such as a knee, elbow, hips, hands, neck, lower back, and/or any other joint, that is affected by osteoarthritis.

In some embodiments, the pharmaceutical composition is effective to reduce inflammation, reduce progression of osteoarthritis, and/or re-differentiate osteoarthritic chondrocytes. In some specific embodiments, the HA and the corticosteroid are synergistically effective to reduce inflammasome-mediated inflammation. In some specific embodiments, the HA and the corticosteroid are synergistically effective to reduce IL-1β. In some specific embodiments, the HA and the corticosteroid are synergistically effective to increase Col2a1.

In some embodiments, the pharmaceutical composition comprises DA, Dex, decanoate, Lac, or a combination thereof. In a non-limiting example, the pharmaceutical composition may comprise or consist of DA and Dex. In a non-limiting example, the pharmaceutical composition may comprise or consist of DA and decanote. In a non-limiting example, the pharmaceutical composition may comprise or consist of DA and Lac. In a non-limiting example, the pharmaceutical composition may comprise or consist of Dex and decanoate. In a non-limiting example, the pharmaceutical composition may comprise or consist of Dex and Lac. In a non-limiting example, the pharmaceutical composition may comprise or consist of Lac and decanoate.

In some embodiments, the pharmaceutical composition comprises a therapeutically-effective amount of a corticosteroid as described anywhere herein, and a therapeutically-effective amount of a lipid as described anywhere herein.

In some embodiments, the therapeutically-effective amount of the corticosteroid is a subclinical dose of the corticosteroid; the subclinical dose reduces the risk of side effects caused by the corticosteroid relative to a clinical dose; and the therapeutically-effective amount of the corticosteroid displays a clinically-relevant effect at treating the osteoarthritis because the corticosteroid and the lipid are synergistically effective to treat the osteoarthritis. In some specific embodiments, the clinical dose is at least 3 milligrams of the corticosteroid, and the subclinical dose is less than 3 milligrams of the corticosteroid. In some very specific embodiments, the clinical dose is at least 3 milligrams of Dxp, and the subclinical dose is less than 3 milligrams of the Dxp.

In some embodiments, the therapeutically-effective amount of the corticosteroid and the therapeutically-effective amount of the lipid are synergistically effective to treat the osteoarthritic joint.

In some embodiments, the therapeutically-effective amount of the corticosteroid and the therapeutically-effective amount of the lipid are synergistically effective to reduce inflammasome-mediated inflammation in the osteoarthritic joint.

In some embodiments, the therapeutically-effective amount of the corticosteroid and the therapeutically-effective amount of the lipid are synergistically effective to reduce IL-1β concentration in the osteoarthritic joint.

In some embodiments, the therapeutically-effective amount of the corticosteroid and the therapeutically-effective amount of the lipid are synergistically effective to reduce matrix MMP13 concentration in the osteoarthritic joint.

In some embodiments, the therapeutically-effective amount of the corticosteroid and the therapeutically-effective amount of the lipid are synergistically effective to increase Col2a1 in the osteoarthritic joint.

In some embodiments, the method comprises administering at least 50 micrograms and up to 5 milligrams of the corticosteroid to the patient. In some specific embodiments, the method comprises administering at least 100 micrograms and up to 2.5 milligrams of the corticosteroid. In some very specific embodiments, the method comprises administering at least 200 micrograms and up to 2 milligrams of the corticosteroid. A dose of about 3 or 4 milligrams of Dxp, for example, is generally effective at treating osteoarthritis of the knee when administered as an intra-articular injection to the knee. The combination of Dxp with a lipid as described herein allows for administration of a reduced amount of the corticosteroid relative to prior art amounts while displaying a similar or superior therapeutic effect thereby reducing the risk of side effects attributable to corticosteroids.

In some embodiments, the administering is local administering in proximity to a joint affected by the osteoarthritis. In some specific embodiments, the administering is selected from topical administering and injecting. In some very specific embodiments, the administering is intra-articular injection.

In some embodiments, the method comprises injecting the pharmaceutical composition into a joint of the patient. In some specific embodiments, the method comprises injecting the pharmaceutical composition into a joint of the patient, wherein the joint is affected by osteoarthritis.

In some embodiments, the joint is selected from the knee, hip, and shoulder. In some specific embodiments, the joint is the knee.

In some embodiments, the method comprises re-injecting a second therapeutically effective amount of the pharmaceutical composition into the joint of the patient 5 to 25 days after the initial injection. In some specific embodiments, the method comprises re-injecting a second therapeutically effective amount of the pharmaceutical composition into the joint of the patient 5 to 15 days after the injecting. The second therapeutically effective amount may be the same or different form the first therapeutically effective amount delivered at the initial treatment. In some embodiments, the second therapeutically effective amount is less than or greater than the first therapeutically effective amount.

In some embodiments, the pharmaceutical composition is effective to decrease PGE₂ concentration in the patient. In some specific embodiments, the pharmaceutical composition is effective to decrease PGE₂ concentration in a joint affected by osteoarthritis in the patient, for example, following injection of the pharmaceutical composition into or adjacent to the joint. In some very specific embodiments, the lipid and the corticosteroid are synergistically effective to decrease PGE₂ concentration in the patient.

In some embodiments, the pharmaceutical composition is effective to decrease inflammasome-mediated inflammation in the patient. In some specific embodiments, the pharmaceutical composition is effective to decrease inflammasome-mediated inflammation in a joint affected by osteoarthritis in the patient, for example, following injection of the pharmaceutical composition into or adjacent to the joint. In some very specific embodiments, the lipid and the corticosteroid are synergistically effective to decrease inflammasome-mediated inflammation in the patient.

In some embodiments, the pharmaceutical composition is effective to decrease IL-1β concentration in the patient. In some specific embodiments, the pharmaceutical composition is effective to decrease IL-1β concentration in a joint affected by osteoarthritis in the patient, for example, following injection of the pharmaceutical composition into or adjacent to the joint. In some very specific embodiments, the lipid and the corticosteroid are synergistically effective to decrease IL-1β concentration in the patient.

In some embodiments, the pharmaceutical composition is effective to increase Col2a1 in the patient. In some specific embodiments, the pharmaceutical composition is effective to increase Col2a1 in a joint affected by osteoarthritis in the patient, for example, following injection of the pharmaceutical composition into or adjacent to the joint. In some very specific embodiments, the lipid and the corticosteroid are synergistically effective to increase Col2a1 in the patient.

In some embodiments, the pharmaceutical composition is effective to inhibit cyclooxygenase in the patient such as COX-1 or COX-2. In some specific embodiments, the pharmaceutical composition is effective to inhibit cyclooxygenase in a joint affected by osteoarthritis in the patient, for example, following injection of the pharmaceutical composition into or adjacent to the joint. In some very specific embodiments, the lipid and the corticosteroid are synergistically effective to inhibit cyclooxygenase in the patient.

In some embodiments, the pharmaceutical composition is effective to inhibit MMP13 in the patient. In some specific embodiments, the pharmaceutical composition is effective to inhibit MMP13 in a joint affected by osteoarthritis in the patient, for example, following injection of the pharmaceutical composition into or adjacent to the joint. In some very specific embodiments, the lipid and the corticosteroid are synergistically effective to inhibit MMP13 in the patient.

In some embodiments, the pharmaceutical composition is effective to re-differentiate osteoarthritic chondrocytes in the patient. In some specific embodiments, the pharmaceutical composition is effective to re-differentiate osteoarthritic chondrocytes in a joint affected by osteoarthritis in the patient, for example, following injection of the pharmaceutical composition into or adjacent to the joint. In some very specific embodiments, the lipid and the corticosteroid are synergistically effective to re-differentiate osteoarthritic chondrocytes in the patient.

The following Examples provide a framework to implement various aspects of the disclosure, and these Examples do not limit any aspect of the disclosure or any patent claim that matures from this patent document.

### EXEMPLIFICATION

The results of these Examples demonstrate that any of the foregoing lipids, corticosteroids, lactate (Lac), and/or other additives that have been discussed herein generally display anti-inflammatory properties that are particularly relevant to osteoarthritis and advantageously display synergy when combined with any of the other lipids, corticosteroids, Lac, or other additives that have also been discussed herein that are commonly used to treat osteoarthritis. Specifically, the results of this Example demonstrate that synergy occurs over a wide range of concentrations of the foregoing lipids, corticosteroids, Lac, and/or other additives described herein. For example, these Examples demonstrate that synergies occur with concentrations of corticosteroids of at least 400 picomolar and up to 20 millimolar, concentrations of lipids of at least 10 micromolar and up to 5 millimolar, concentrations of Lacs of at least 20 millimolar and up to 50 millimolar, and any concentration of any additive described herein.

### Example 1. Decanoic Acid (DA) Inhibits IL-1β-Induced Prostaglandin E₂ (PGE₂) Release From Normal Human Chondrocytes

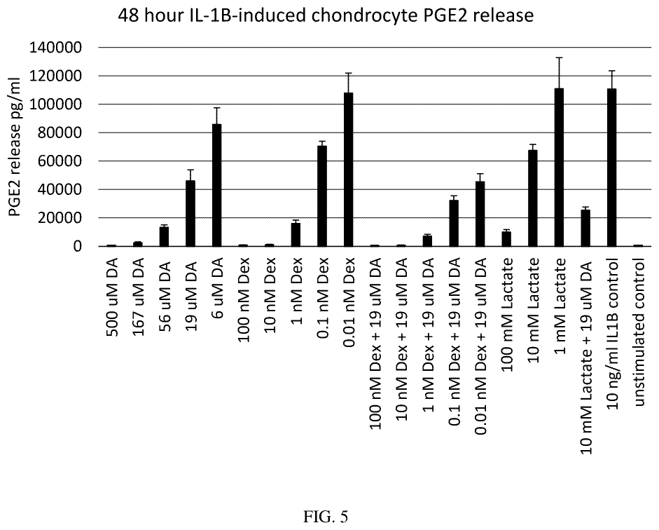

Mechanical stress and inflammation are thought to sustain catabolic cycles of extracellular matrix deterioration during osteoarthritis. In particular, PGE₂ correlates with increased protease production and discomfort or pain in osteoarthritis. Monolayers of normal human chondrocytes were stimulated with 500 picograms per milliliter IL-1β for 48-hours and then PGE₂ release was evaluated by a competitive EIA following exposure to dose-responses of DA. FIG. 5 depicts the concentration of PGE₂ present in the supernatant of normal human chondrocyte cell cultures that were stimulated with interleukin 1-beta (IL-1β) for 48 hours in the presence of varying concentrations of DA, dexamethasone (Dex) and Lac. The combination of DA and Dex displayed an additive response, and the combination of Lac and DA also displayed an additive response. Additionally, DA and Lac dose-dependently inhibit IL-1β-induced PGE₂ release and the DA response is preserved in combination with both Dex and Lac. FIG. 5 demonstrates that treatment with DA suppresses PGE₂ release from cytokine-activated chondrocytes in a dose-dependent manner with complete attenuation (100 percent inhibition) observed at a final concentration of 500 micromolar. Furthermore, significant inhibition of PGE₂ release was observed at final concentrations of 6 micromolar DA (p-value≤0.05) versus medium controls. FIG. 5 also demonstrates that treatment with Dex suppresses PGE₂ release from cytokine-activated chondrocytes in a dose-dependent manner with complete attenuation (100 percent inhibition) observed at a final concentration of 10 nanomolar. Statistical significance was observed at concentrations of 100 picomolar. Lac also displayed a statistically significant effect at concentrations of 10 millimolar and above. The results demonstrate that metabolic and/or oxidative management of chondrocytes via DA treatment is beneficial throughout inflammatory, cartilage-degrading phases of osteoarthritis.

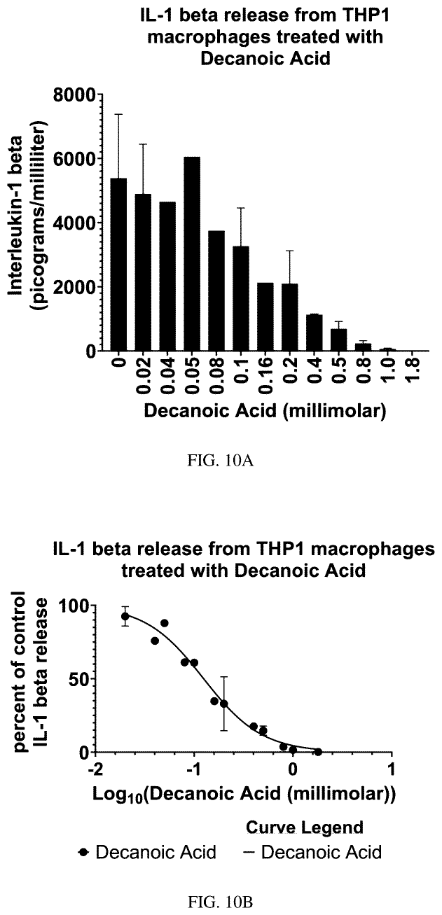

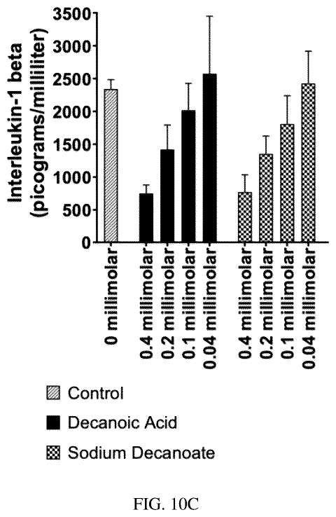

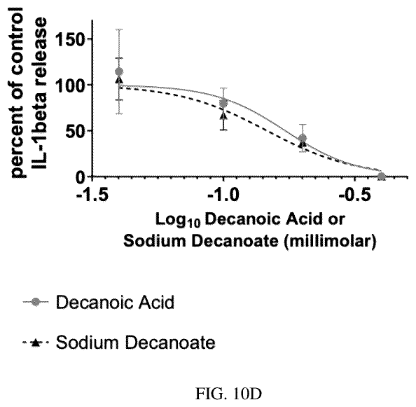

The activity of DA working solutions prepared in either saline or lactated ringer's (LR) isotonic solutions was assessed. Normal human chondrocyte monolayers were stimulated with IL-1β for 24 hours and PGE₂ release was measured by a competitive EIA after exposure to doses of DA or decanoate, ranging from 100 to 6.25 micromolar, prepared in culture medium. In addition, concentrated decanoate working solutions in saline or LR were prepared and added to the cultures at a final concentration of 25 micromolar. PGE₂ release was then compared to matching points on the DA or decanoate medium as well as saline and LR controls. DA and decanoate inhibited IL-1β-induced PGE₂ release from chondrocytes similarly with a relative potency of decanoate as compared to DA calculated at 0.92 (95 percent confidence interval of 0.80-1.06) (FIG. 10C). Comparing the activity of the 25 micromolar doses of the test samples revealed that inhibitions of 62.4+/−3.8%, 62.3+/−0.98%, 63.2+/−4.1%, and 65.2+/−2.7% in PGE₂ release were observed for DA prepared in medium, decanoate prepared in medium, decanoate prepared in saline, and decanoate prepared in LR (FIG. 10D). These data show that DA and decanoate behave similarly.

### Example 2. DA Enhances the Potency and Efficacy of Dex at Inhibiting IL-1β-Induced PGE₂ Release From Normal Human Chondrocytes

Corticosteroids are potent anti-inflammatory medications used to treat osteoarthritis-related pain and loss of function. Furthermore, intraarticular injections of corticosteroids are commonly used to relieve the pain associated with moderate-to-severe osteoarthritis and to inhibit the production of inflammatory mediators that enhance catabolism. Repeated use of corticosteroids, however, may exacerbate disease and promote further cartilage loss.

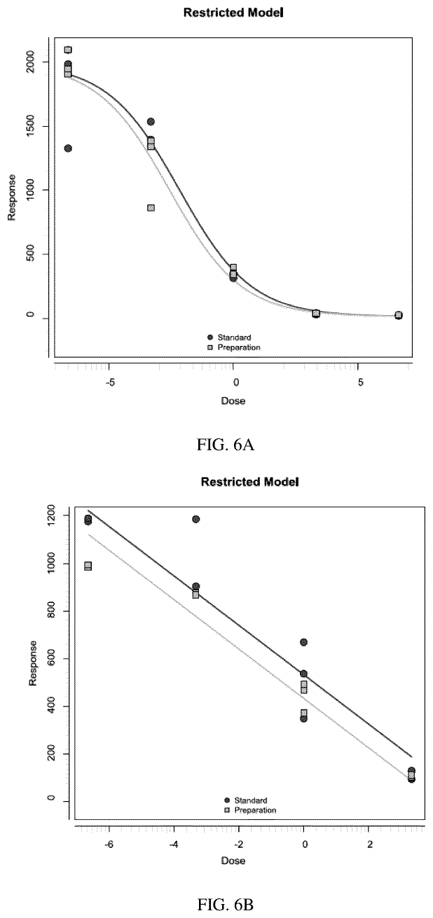

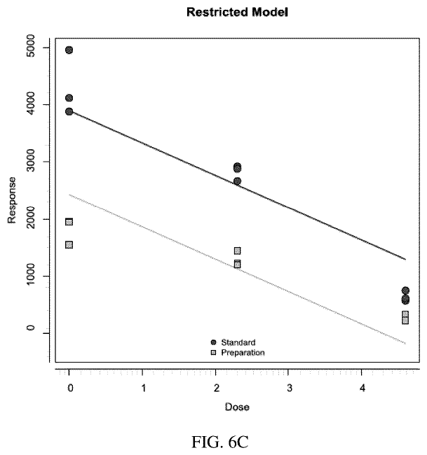

IL-1β activated monolayers of normal human chondrocytes were exposed to increasing concentrations of Dex in the absence or presence of a constant concentration of DA. After 48-hours, PGE₂ release was evaluated by competitive EIA, and relative potency was calculated versus dose responses of Dex alone. Final concentrations of 2.0, 12.5, and 50 micromolar DA potentiated the inhibition of PGE₂ release in the model, with elevated reductions in the detectable amount of PGE₂ measured in the supernatants. When relative potency was calculated versus Dex-only response, curves via parallel line analysis, potency was enhanced by 1.32-fold, 1.93-fold, and 13.33-fold, respectively (FIGS. 6A, 6B, and 6C; Table 1). Moreover, these represent super-additive responses versus the 1.24-fold, 1.43-fold, and 5.26-fold changes in potency expected based on the inhibition of PGE₂ observed for DA alone in the assays. Together these data demonstrate that DA enhances the anti-inflammatory activity of Dex allowing for the administration of lower doses that would minimize the deleterious effects observed clinically with repeated injections.

TABLE 1
The Combination of Dex and DA displays a Super-Additive Effect
at Inhibition PGE₂ Release from Normal Human Chondrocytes.

| | Actual | Expected |
|---|---|---|
| Final | Relative | Relative |
| Concentration | Potency of | Potency of |
| of DA | Combination | Combination |
| 2.0 micromolar | 1.32 | 1.24 |
| 12.5 micromolar | 1.93 | 1.43 |
| 50.0 micromolar | 13.33 | 5.26 |

FIGS. 6A-6C are parallel-line bioassay graphs depicting shifts in potency for dose-responses of Dex, as measured by reduced IL-1β-induced PGE₂ release, as compared to Dex dose-responses combined with 2.0, 12.5, and 50 micromolar final concentrations of DA (FIGS. 6A, 6B, and 6C respectively). IL-1β-activated monolayers of normal human chondrocytes were exposed to increasing concentrations of Dex in the absence or presence of a constant concentration of DA (as indicated in FIGS. 6A, 6B, and 6C respectively). After 48 hours, PGE₂ release was evaluated by competitive enzyme immunoassay (EIA), and relative potency was calculated versus Dex alone. Parallel line analysis demonstrates that DA dose-dependently increased the potency of Dex by 1.32-fold, 1.93-fold, and 13.33-fold, respectively (FIGS. 6A, 6B, and 6C).

### Example 3. DA Protects Against Reductions in SRY-Box Transcription Factor 9 (SOX9) Induced by IL-1β in Normal Human Chondrocytes

IL-1β is a pro-inflammatory cytokine produced by the inflammasome that is linked to joint inflammation and cartilage destruction. One of the primary mechanisms by which IL-1β is thought to promote damage is the suppression of SOX9 expression in chondrocytes, which causes these cells to dedifferentiate into pathogenic phenotypes.

Normal human chondrocyte monolayers were treated with DA dose responses and then exposed to IL-1β for 72-hours. Total RNA was then isolated, and relative expression of SOX9 was calculated using quantitative reverse transcript real time polymerase chain reaction (qRT-PCR) versus controls, normalized to glyceraldehyde-3-phosphate dehydrogenase (GAPDH). DA inhibited the loss of SOX9 transcription caused by IL-1β in a dose-dependent manner. When monolayers of normal human chondrocytes were stimulated with IL-1β, SOX9 expression decreased 5-fold compared to unstimulated controls. Treatment with 500, 50, and 10 micromolar concentrations of DA resulted in relative expressions of 2-fold, 4-fold, and 7-fold, respectively. These findings demonstrate that DA treatment of chondrocytes prevents the loss of SOX9 caused by inflammation.

### Example 4. Lac Exhibits Anti-Inflammatory and Cartilage-Protective Properties in Chondrocyte in Vitro Culture

Lac is commonly thought to be simply an end-product of anaerobic glycolysis; however, it may also have immunomodulatory effects and promote protein modifications that alter gene expression. The goal of this experiment was to determine how Lac supplementation affects early-phase, inflammatory, and late-stage redifferentiation in chondrocyte models. To establish how Lac may influence inflammation early in osteoarthritis, monolayers of normal human chondrocytes were exposed to Lac dose responses of up to 100 millimolar, concurrently with 0.5 nanograms per milliliter IL-1β for 48 hours. The release of PGE₂ was then quantified using competitive EIA. To investigate how Lac affects transcriptional programs in dedifferentiated osteoarthritic chondrocytes, monolayer cultures and micromass pellets were treated for 14 days with 100 millimolar final concentrations of Lac, with and without 10 nanograms per milliliter transforming growth factor beta 3 (TGF-β3). Total RNA was then isolated, and relative expression of SOX9 or Col2a1 was calculated using qRT-PCR, normalized to GAPDH. Lac exhibited a dose-dependent ability to inhibit the release of PGE₂ from normal human chondrocytes stimulated with IL-1β, with complete inhibition observed at 100 millimolar. Lac may also have beneficial effects on osteoarthritic chondrocytes; 100 millimolar final Lac concentrations increased the relative expression of SOX9 and Col2a1 by 8-fold and 1600-fold, respectively, in monolayer cultures. Micromass pellets showed an 11-fold increase in collagen. Furthermore, adding Lac with TGF-β3 to micromass cultures increased SOX9 and Col2a1 by 9-fold and 88-fold, respectively. These findings demonstrate that Lac treatment of chondrocytes promotes metabolic management, which are beneficial during inflammatory stages of osteoarthritis and promote transcriptional programs that preserve cartilage.

### Example 5. DA Inhibits IL-1β-Induced Monocyte Chemoattractant Protein 1 (MCP1) in Human Synoviocytes Isolated From a Patient With Osteoarthritis (Osteoarthritic-Synoviocytes)

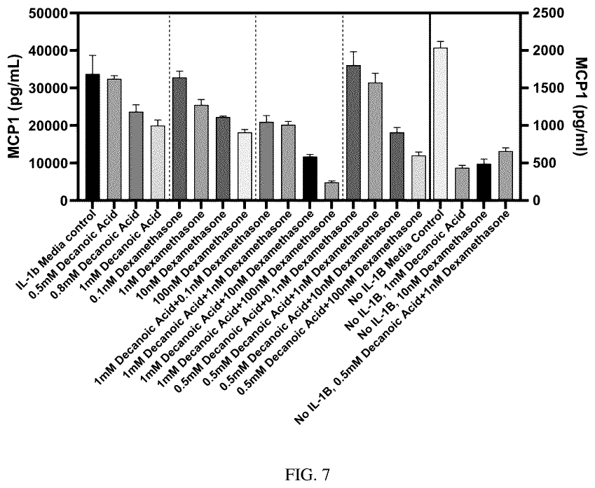

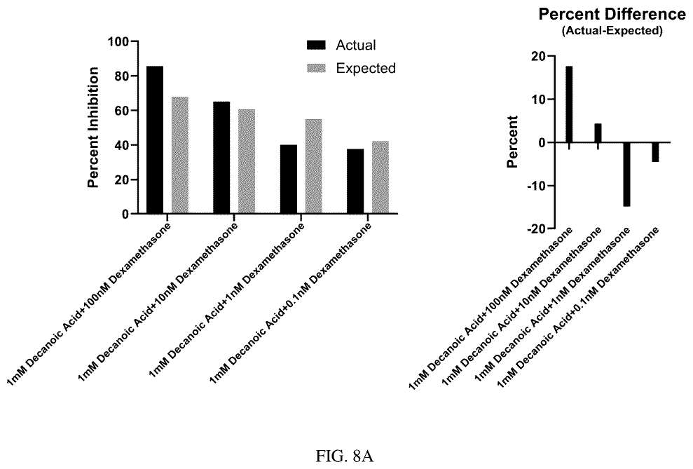

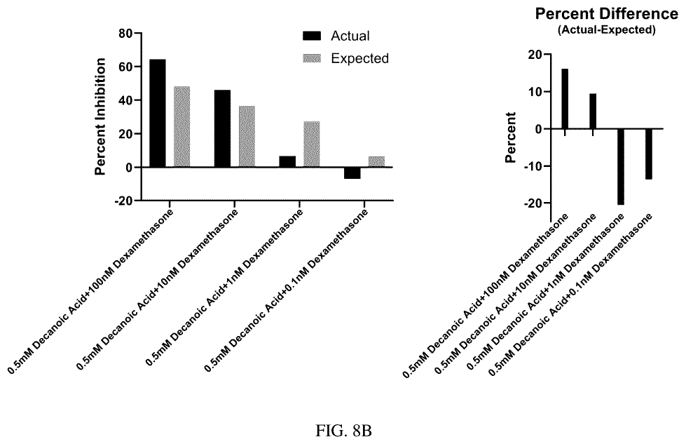

Osteoarthritic-synoviocytes stimulated with IL-1β at 0.5 nanograms per milliliter displayed a reduction in MCP1 in a dose dependent manner for synoviocytes treated with 0.5, 0.8, or 1 millimolar DA, 0.1, 1, 10, or 100 nanomolar Dex, and combinations thereof relative to controls as illustrated in FIG. 7. The left y-axis of FIG. 7 corresponds to data to the left of the solid black line (“IL-1β Media Control” to “0.5 DA_100 Dex”), and the right y-axis of FIG. 7 corresponds to data to the right of the solid black line (“No IL-1β media control” to “Unstim 0.5 DA_1 Dex”). Even in the unstimulated osteoarthritic-synoviocytes (right y-axis, bars to the right of solid black line), reductions in MCP1 were observed relative to the unstimulated control. MCP1 attracts monocytes, and thus, increases inflammation. Synergy was observed at concentrations greater than 1 nanomolar dexamethasone as illustrated in FIGS. 8A and 8B. The bar graphs in the left panel of each of FIGS. 8A and 4B display the comparisons, and the bar graphs in the right panel of each of FIGS. 8A and 8B depict differences between the expected, additive effects and the actual, observed effects. The ability of DA and Dex to synergistically reduce MCP1 expression therefore suggests a synergistic effect on inflammation generally and inflammasome-mediated inflammation specifically.

### Example 6. DA Inhibits IL-18-Induced PGE₂ and Prostaglandin D2 (PGD₂) in Human Synoviocytes Isolated From a Patient With Osteoarthritis (Osteoarthritic-Synoviocytes)

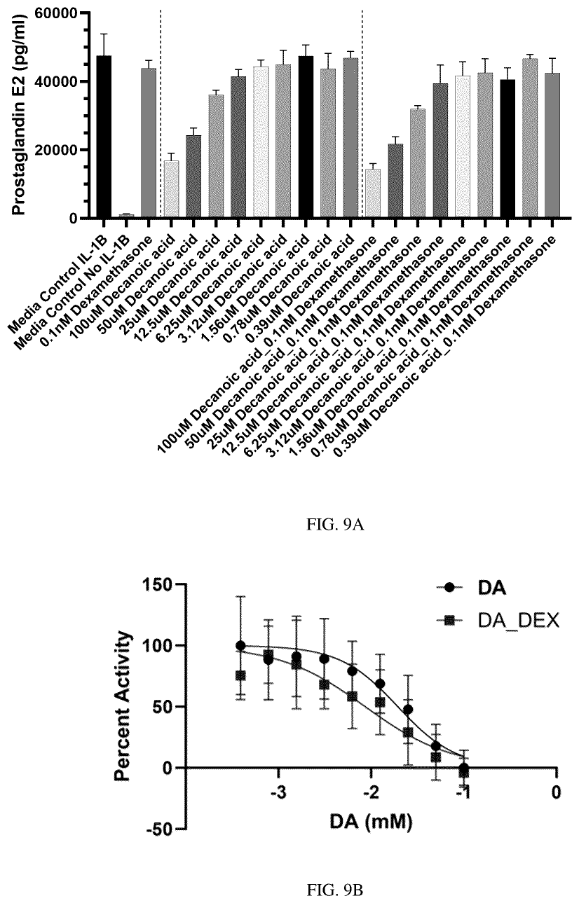

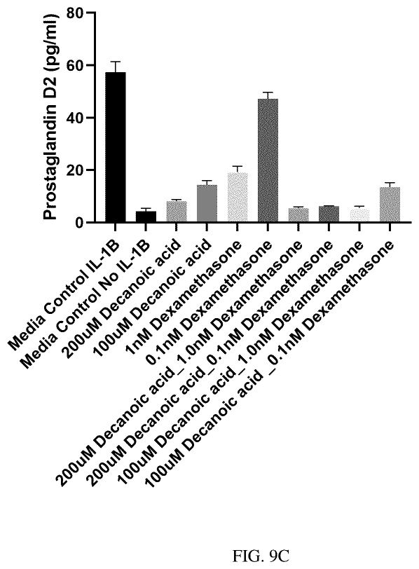

Osteoarthritic-synoviocytes stimulated with IL-1β at 0.5 nanograms per milliliter displayed an induction in PGE₂ and PGD₂ release into the supernatant that was inhibited by DA at concentrations above about 1 micromolar with the addition of 100 picomolar Dex displaying an additive effect as illustrated in FIGS. 9A, 9B, and 9C. The IC50 for DA alone was 19.42 nanomolar, and the IC50 for DA in the presence of 100 picomolar Dex was 8.624 nanomolar (FIG. 9B). The 100 picomolar Dex thereby increased the potency of DA by 2.2× in this assay.

### Example 7. DA Inhibits IL-1β Release From Human Macrophage-Like Cells

The proinflammatory cytokine, IL-1β, is released from cells upon activation of the inflammasome, a multi-protein proinflammatory complex. A two-hit model of inflammasome activation in THP1 macrophages was developed that entails an overnight stimulation with LPS followed by a second proinflammatory stimulus, extracellular ATP, which drives inflammasome assembly and subsequent IL-1β release from the cells into the media.

In three independent experiments, THP1 macrophages stimulated by the two-hit model of inflammasome activation released IL-1β in control conditions. To define IC50s, two-hit-stimulated THP1 macrophages were treated with dilutions of DA and Dex phosphate and IL-1β release was measured. The concentration ranges tested were 0.02 to 1.8 millimolar DA, and the calculated IC50 concentration was 0.1246 millimolar for DA as illustrated in FIGS. 10A and 10B. These data were plotted on a logarithmic scale, which was used to determine the IC50 of DA equal to 0.1246 millimolar in the assay. These experiments were repeated to compare the effectiveness of DA to sodium decanoate to inhibit IL-1β in this model. FIGS. 10C and 10D show that DA and sodium decanoate similarly inhibit IL-1β. These data were plotted on a logarithmic scale, and in these experiments, the IC50 values were calculated as 0.1719 millimolar for DA (grey circles) and 0.1464 millimolar for sodium decanoate (black triangles) in the assay. For Dex phosphate, the concentration ranges tested were 1 picomolar to 10 micromolar, and the calculated IC50 concentration was 46.02 nanomolar for Dex phosphate as illustrated in FIGS. 11A and 11B. These data were plotted on a logarithmic scale, which was used to determine the IC50 of Dex phosphate (Dxp) equal to 46.02 nanomolar in the assay. DA also reduced the expression of interleukin-18, tumor necrosis factor, interleukin-8, and interleukin-6 in a dose dependent manner with an IC50 that was about the same for each cytokine (data not shown).

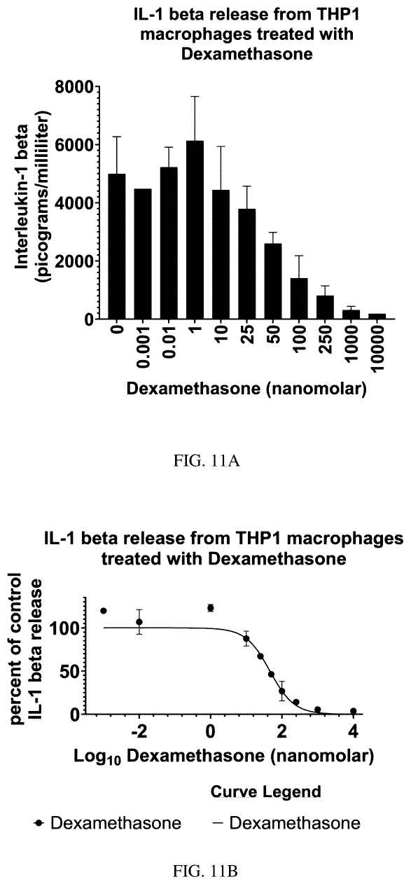

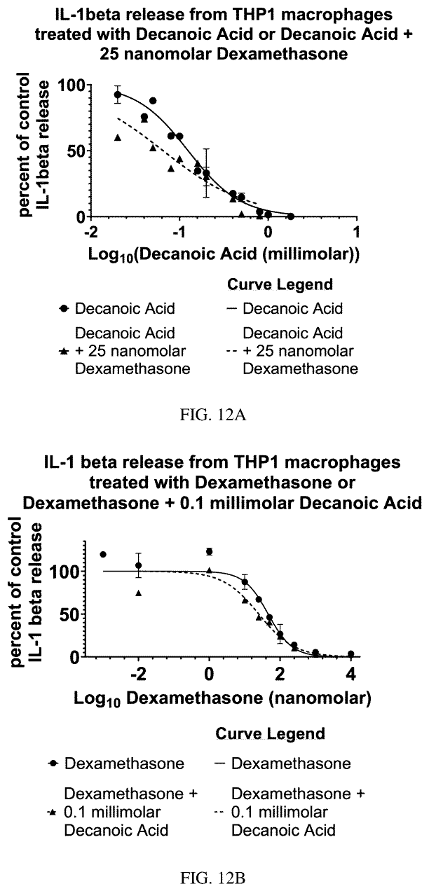

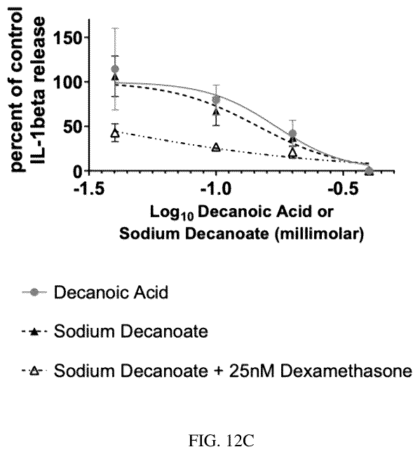

The dose-response curve for DA with the addition of 25 nanomolar Dex was also measured. DA alone displayed an IC50 equal to 0.1246 millimolar; whereas the addition of 25 nanomolar Dex lowered the IC50 to 0.065 millimolar, which corresponds to an increased DA potency of 1.917× (FIG. 12A). The dose-response curve for Dex with the addition of 100 micromolar DA was also measured. Dex alone displayed an IC50 of 46.02 nanomolar, whereas the addition of 100 micromolar DA lowered the IC50 to 23.37 nanomolar, which corresponds to an increased Dex potency of 1.969× (FIG. 12B). FIG. 12C compares the calculated IC50 value of sodium decanoate and sodium decanoate with the addition of 25 nanomolar Dex. The addition of Dex decreased the IC50 of sodium decanoate from 0.1464 millimolar to 0.03152 millimolar in the assay, an increase in potency of 4.6×. These results demonstrate that the addition of a medium-chain fatty acid to a corticosteroid formulation can reduce the amount of the corticosteroid to be administered by about 50 percent, which is likely to significantly reduce the side effects caused by administering the corticosteroid.

FIG. 13 shows box plots of IL-1β release from 2-hit inflammasome stimulated THP1 treated with 0, 25, 50, 100, or 150 micromolar sodium decanoate diluted either in saline (left panel) or in LRs solution (right panel).

### Example 8. The Combination of DA and Dex Synergistically Decreases Matrix Metalloproteinase 13 (MMP13) Expression in Osteoarthritic Fibroblast-Like Chondrocytes

Osteoarthritic fibroblast-like chondrocytes were cultured for 14 or 28 days in the presence of either 0.5 millimolar DA, 0.1 micromolar Dex, or both 0.5 millimolar DA and 0.1 micromolar Dex (DA+Dex). Media was exchanged every 2-3 days. Chondrocytes cultured for 14 days, or 28 days were analyzed by qRT-PCR to assess MMP13 expression.

MMP13, which is also known as collagenase-3, is an endopeptidase that plays a crucial role in the degradation and remodeling of the extracellular matrix. Under normal physiological conditions, MMP13 is tightly regulated and involved in the turnover of collagen. In osteoarthritis, however, the overexpression and dysregulated activity of MMP13 leads to excessive collagen degradation and cartilage destruction.

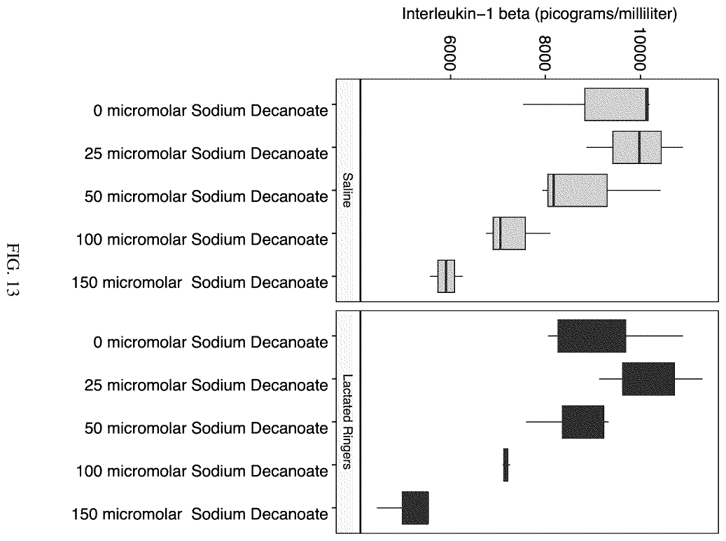

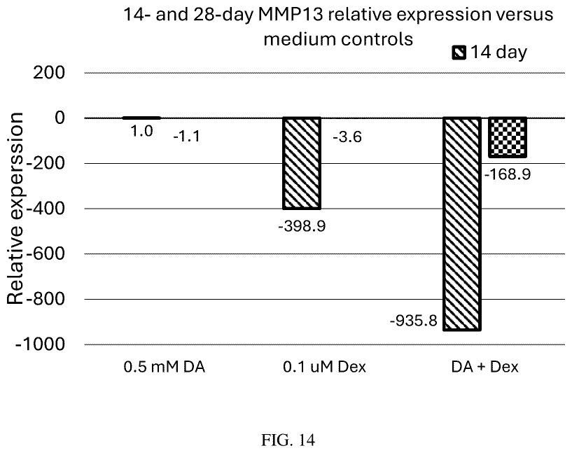

Change in MMP13 expression as calculated by delta-delta CT relative to GAPDH expression demonstrated unexpected synergy between 0.5 millimolar and 0.1 micromolar Dex as shown in FIG. 14. The synergy displayed at both day 14 and day 28 displayed a super-additive effect. These results suggest that the combination of DA and Dex can synergistically reverse collagen degradation in osteoarthritis.

### Example 9. An Exemplary Aqueous Formulation

An exemplary aqueous formulation of the invention comprises the following:

TABLE 2
Exemplary Formulation.

| Ingredient | Concentration | |
|---|---|---|
| DA | 0.5 | millimolar |
| Dex or Dxp | 0.5 | millimolar |
| Sodium cation | 130 | millimolar |
| Chloride anion | 109 | millimolar |
| Lac | 28 | millimolar |
| Potassium cation | 4 | millimolar |
| Calcium cation | 1.5 | millimolar |

The pH of the exemplary formulation of Table 2 is generally adjusted to about 6.5 and buffered by the Lac and/or phosphate buffer.

### Example 10. Lac Alters Remodeling Transcriptional Programs Induced by Transforming Growth Factor Beta 1 (TGF-β1) and Interleukin-1 Beta (IL-1β) in Human Dermal Fibroblasts (NHDF)

Cutaneous wound healing is a dynamic process that restores physiologic structure and barrier function. Dysregulation of the highly orchestrated events involved can lead to the formation of chronic wounds, which can cause delays in closure and create additional burdens for management. Fibroblasts migrating into the wound bed perform important roles during remodeling such as depositing extracellular matrix proteins and releasing proteases that resolve the provisional matrix to a mature scar. A greater understanding of fibroblast function during this process could aid in developing new therapy options. Once considered strictly a waste product of glycolysis, Lac has emerged as a critical metabolite and signaling molecule. Furthermore, studies indicate that Lac can build to high levels in the wound bed (5-40 mM).

In order to investigate the effects of Lac on TGF-β1- and IL-1β 1B-induced transcriptional programs in normal human dermal fibroblasts (NHDF), qRT-PCR was used to evaluate changes in the messenger RNA (mRNA) levels of key remodeling markers (i.e. Elastin (ELN), matrix metalloproteinase 3 (MMP3), matrix metalloproteinase 9 (MMP9), matrix metalloproteinase 12 (MMP12), Collagen 1a1, and Collagen 4a1)) in NHDF treated with 100 mM sodium Lac and then activated with 10 ng/ml TGF-β1 or 0.1 ng/ml IL-1β. To serve as a control, cells were also treated with 0.1 mM Dex-21-phosphate (Dxp or Dex). After 24 hours, total mRNA was isolated and delta-delta Cp relative expression was calculated versus untreated medium controls, normalized to the housekeeping gene, GAPDH. Statistical analysis was then conducted via t-test.

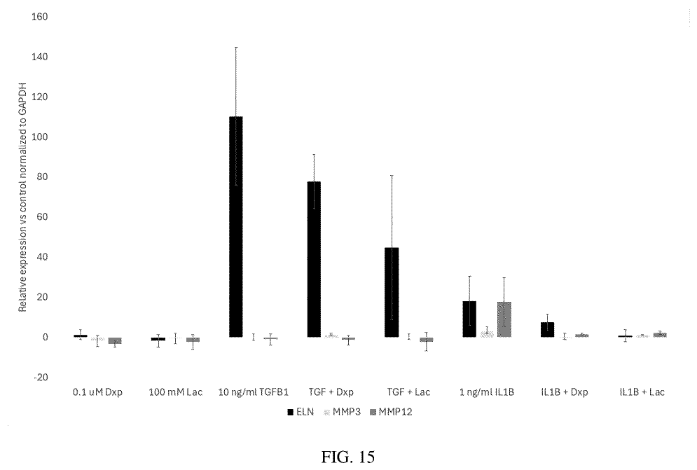

As demonstrated in FIG. 15, it was found that 100 mM Lac treatment inhibited the increased relative expression of ELN observed following activation with TGF-β1 or IL-1β [TGF-β1=110±35, TGF-β1+Lac=45+36 (p-value=0.077), IL-1β=18±12, IL-1β+Lac=0.86±3 (p-value=0.063); n=3]. Dxp also trended to reduce cytokine-induced ELN expression [TGF-β1+Dxp=78±14 (p-value=0.17), IL-1β+Dxp=7.8+4 (p-value=0.14)]. It was also observed that both Lac and Dxp exhibited an ability to reduce IL-1β-induced increases in MMP3 [IL-1β=3.5±1.7, IL-1β+Lac=1.5±0.8 (p-value=0.097), IL-1β±Dxp=0.6±1.5 (p-value=0.64)] and MMP12 [IL-1β=17.8±12.3, IL-1β+Lac=2.2±0.8 (p-value=0.085), IL-1β+Dxp=1.7±0.6 (p-value=0.079)].

The data demonstrates that treatment of NHDF with high levels of Lac can mimic changes in pro-inflammatory remodeling transcriptomes observed for the glucocorticoid, Dxp. Furthermore, increased Lac is anti-inflammatory and thus redirects the healing process accordingly. Moreover, timely metabolic management of the wound bed with Lac may help stabilize redox states and minimize tissue damage.

### Example 11. Relative Expression of SOX9 and Runt-Related Transcription Factor 2 (RUNX2) in Low and High Passage Osteoarthritic Chondrocytes

Examples of the present disclosure were conducted to evaluate the expression of key chondrocyte markers in the presence of test compounds. High passage and low passage chondrocytes were prepared where in both cases, primary human chondrocytes were cultured as confluent monolayers on tissue culture plates. For the high passage chondrocytes, fibroblast-like human osteoarthritic chondrocytes were used. The cells were cultured for an extended period before investigation (passage 13). As a result, these cells become fibroblast-like, i.e. marked by low expression of chondrocyte markers. On the other hand, the low passage human chondrocytes (passage 3-4) retained expression of key chondrocyte markers.

The high and low passage chondrocytes were cultured for 7, 14 or 28 days in the presence of either 50-100 μM final concentrations of DA or Dec, 28-100 mM D/L of sodium Lac, 0.1 μM of Dex 21-phosphate (Dxp or Dex), 100 μM α-Lipoic acid (LA), and combinations thereof including DA/Dec+Dxp, DA/Dec+Lac, Dxp+Lac, DA/Dec+Dxp+Lac, LA+DA/Dec, LA+Dxp, LA+Lac, and LA+DA/Dec+Dxp. When indicated, the cells were also cultured with TGF-β3 at a final concentration of 10 ng/ml. TGF-β3 is known to induce chondrogenesis. However, elevated levels and dysregulation of TGF signaling pathways are implicated in the pathogenesis of osteoarthritis.

Media was exchanged bi-weekly. Chondrocytes cultured for 7 days, 14 days, or 28 days were analyzed to assess SOX9 and RUNX2 expression. SOX9 is a transcription factor vital to the development and maintenance of cartilage and is required for chondrogenesis. SOX9 promotes chondrocyte survival and transcriptionally activates cartilage-specific genes. SOX9 is repressed in hypertrophic chondrocytes (i.e. terminally differentiated osteoblast-like cells). RUNX2 is a transcription factor essential for osteoblast differentiation and terminal chondrocyte maturation and is up-regulated in osteoarthritic chondrocytes.

The data is presented as GAPDH normalized relative expression of key markers versus unstimulated controls. Results are depicted in FIGS. 15 to 20.

For high passage, dedifferentiated osteoarthritic chondrocytes stimulated with TGF-β3 and cultured for 14 days, DA, LA, and Lac treatment increased SOX9 transcription. It was also discovered that LA and Lac together further increase SOX9 expression.

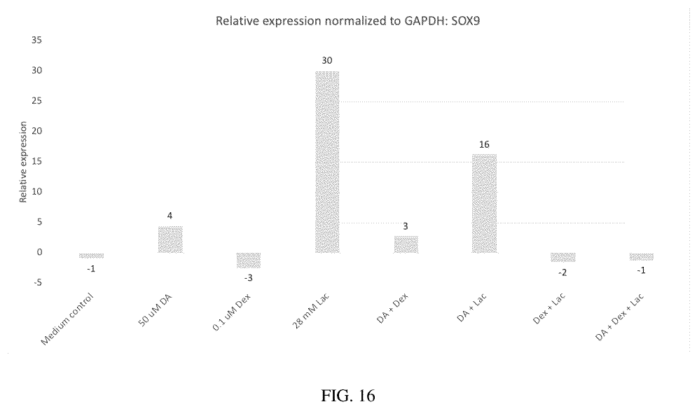

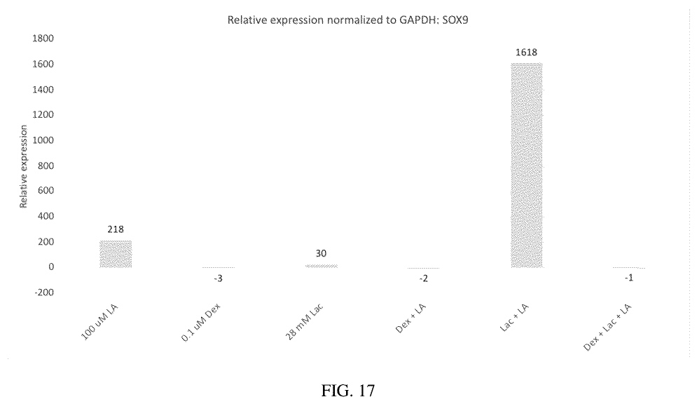

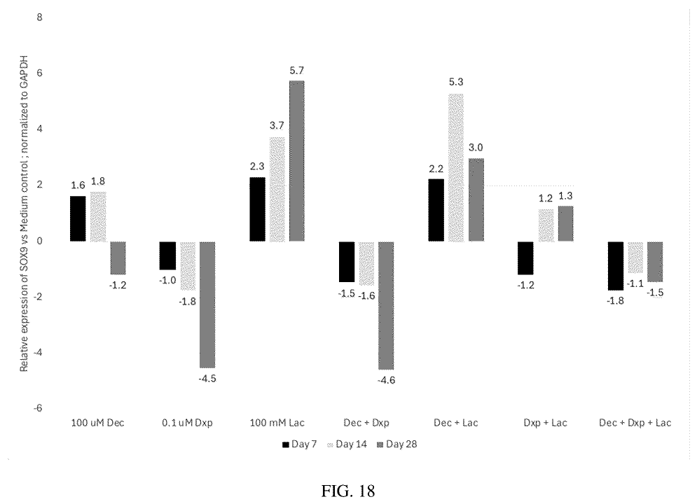

FIGS. 16 and 17 depict that DA, Lac, LA, and LA+Lac increase SOX9 expression in the osteoarthritic chondrocytes. Compared to the high passage chondrocytes of FIGS. 16 and 17, a similar SOX9 response was observed in the low-passage osteoarthritic chondrocytes of FIG. 18. The SOX9 expression is provided at 7, 14, and 28 days of culturing. Additionally, a temporal increase was observed particularly for Lac treatment. Lac also appears to counteract Dxp-induced reductions in SOX9. It was additionally observed that Lac treatment temporally reduced RUNX2 expression in low-passage chondrocytes. Finally, the relative expression of SOX9 versus RUNX2 in low passage osteoarthritic chondrocytes was prepared. The results display that Lac skews the ratio of SOX9 to RUNX2 and long-term exposure to a combo of compounds counteracts the inverse ratio induced by Dxp, with a temporal increase observed.

Dedifferentiated osteoarthritic chondrocytes treated with TGF-β3 exhibit: (1) increased expression of SOX9 when treated with DA/Dec or Lac; (2) decreased expression of SOX9 when treated with Dxp; (3) Dxp-induced reduction in SOX9 rescued by DA/Dec; (4) LA also increases SOX9 relative expression; and (5) Lac+LA combination potentially results in a super-additive response. Additionally, Low passage osteoarthritic chondrocytes exhibited: (1) a temporal increase in SOX9 when treated with Lac; (2) a temporal decrease in expression of SOX9 when treated with Dxp; (3) a decrease in SOX9 rescued by Dec+Dxp+Lac triple treatment; (4) a temporal decrease in RUNX2 with Lac treatment that may be enhanced by the triple combination treatment; (5) the ratio of SOX9/RUNX2 temporally increased by Lac; (6) the ratio of SOX9/RUNX2 was reduced by Dxp; and (7) the ratio reduction by Dxp was rescued by the triple combination.

### Example 12. Relative Expression of MMP13 in Low and High Passage Osteoarthritic Chondrocytes

MMP13 is part of a family of zinc-dependent proteolytic enzymes responsible for the degradation of extracellular matrix (ECM). The preferred target of MMP13 is type II collagen; a primary component of articular cartilage. MMP13 is associated with the destruction of cartilage observed in osteoarthritis and MMP13 release from chondrocytes can be induced by pro-inflammatory cytokines such as IL-1β. RUNX2 binding to the promoter element of MMP 13 is vital to expression. Overexpression of SOX9 can down-regulate MMP13 expression by hypertrophic chondrocytes.

Similar to Example 1, high and low passage chondrocytes were cultured for 7, 14, or 28 days in the presence of either 50-100 μM final concentrations of DA or decanoate (Dec), 28-100 mM D/L of sodium Lac, 0.1 μM of Dex 21-phosphate (Dxp or Dex), and combinations thereof including DA/Dec+Dex/Dxp, DA/Dec+Lac, Dex/Dxp+Lac, and DA/Dec+Dex/Dxp+Lac.

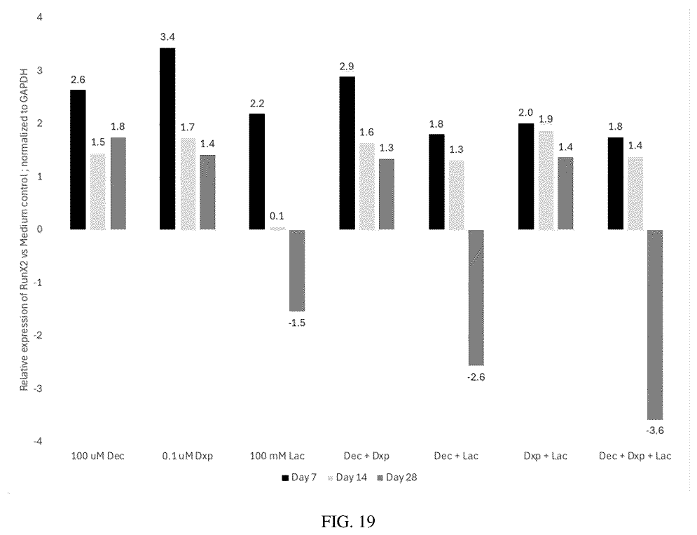

FIG. 19 displays the relative expression of RUNX2 versus a control normalized to GAPDH in low passage osteoarthritic chondrocytes treated with varying compositions. The RUNX2 expression is provided at 7, 14, and 28 days of culturing. It was observed that Lac treatment temporally reduced RUNX2 expression in low-passage chondrocytes.

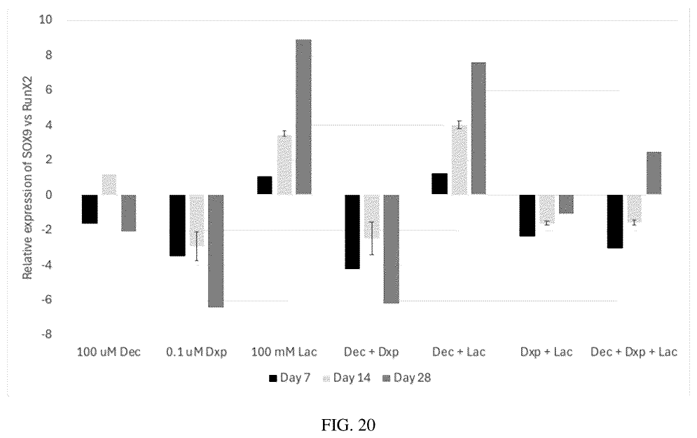

Displayed in FIG. 20 is the temporal analysis of the relative expression of SOX9 versus RUNX2 in low passage osteoarthritic chondrocytes treated with varying compositions. The temporal analysis is provided at 7, 14, and 28 days of culturing. Lac skews the ratio of SOX9 to RUNX2 and long-term exposure to a combo of compounds counteracts the inverse ratio induced by Dxp, with a temporal increase observed.

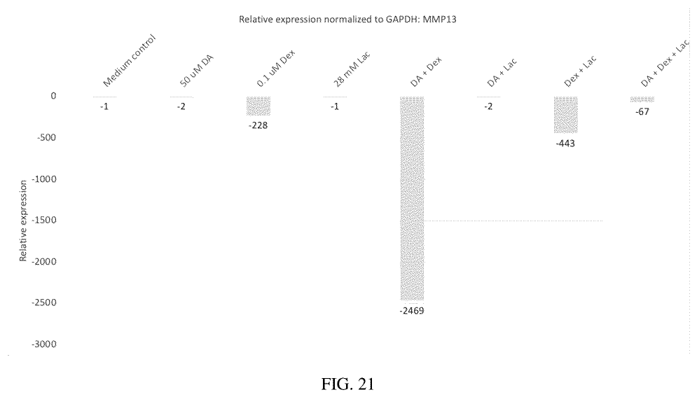

As presented in FIG. 21, in high passage, dedifferentiated osteoarthritic chondrocytes cultured for 14 days, Dex treatment reduced MMP13 transcription. Additionally, combining Dex with DA or Lac enhanced the response versus DA and Lac alone.

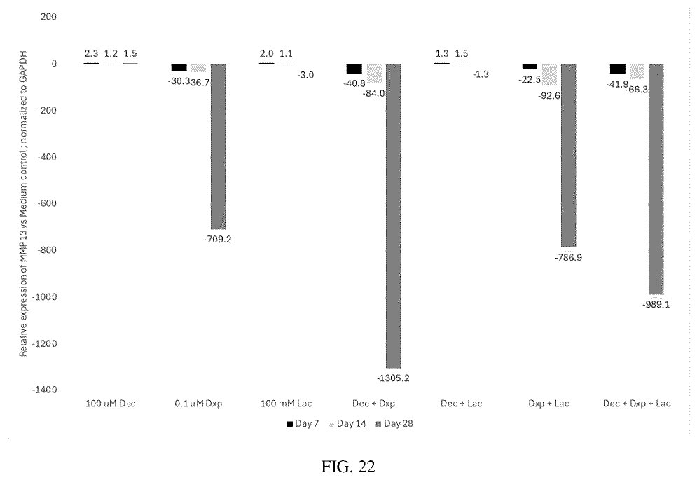

With reference to FIG. 22, which is directed to temporal analysis of low passage osteoarthritic chondrocytes, a similar response MMP13 to the high passage osteoarthritic chondrocytes was seen in low passage osteoarthritic chondrocytes. The temporal analysis is provided at 7, 14, and 28 days of culturing. As depicted, Dxp treatment of low passage osteoarthritic chondrocytes reduce MMP13 transcription and combining Dxp with Dec or Lac enhances the MMP13 reduction compared to Dec and Lac alone. Additionally, it was discovered that longer dosing regimens demonstrated the strongest response.

Thus, dedifferentiated and low passage osteoarthritic chondrocytes exhibit: (1) reduced expression of MMP13 following treatment with Dex (aka Dxp); (2) reduced expression of MMP13 by combining Dex with DA or Dec; and (3) enhanced response with longer periods of exposure.

### Example 13. Relative Expression of Aggrecan (ACAN) and Col2a1 in Low Passage Osteoarthritic Chondrocytes

ACAN and Col2a1 are critical components of healthy articular cartilage. ACAN is a proteoglycan that helps give cartilage its ability to withstand compressive loads and Col2a1 is a fibrillar collagen found in articular cartilage critical to overall structure. A reduction of these proteins is a hallmark of osteoarthritis and accelerates joint damage.

Similar to Examples 11 and 12, low passage chondrocytes were cultured for 7, 14 or 28 days in the presence of either 50-100 μM final concentrations of DA or decanoate (Dec), 28-100 mM D/L of sodium Lac, 0.1 μM of Dex 21-phosphate (Dxp or Dex), and combinations thereof including DA/Dec+Dex/Dxp, DA/Dec+Lac, Dex/Dxp+Lac, and DA/Dec+Dex/Dxp+Lac.

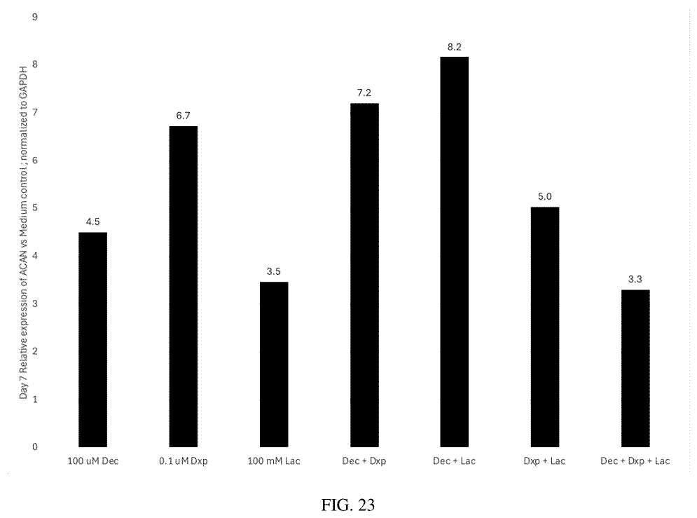

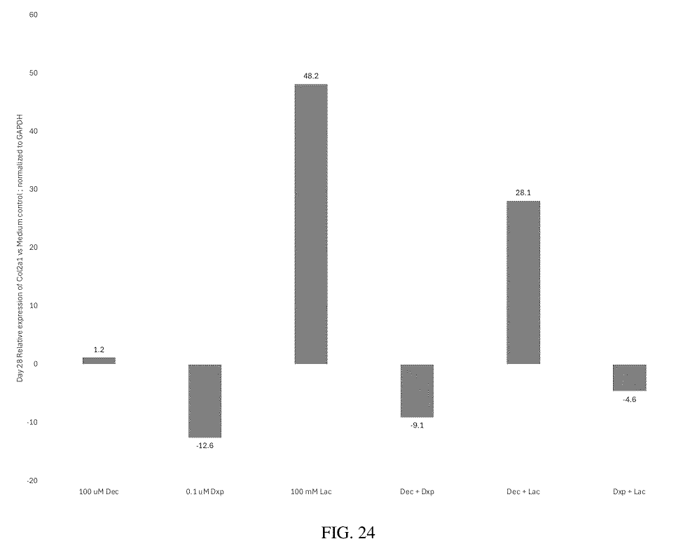

FIGS. 23 and 24 depict the results. As demonstrated in FIG. 23, directed to day 7 relative expression of ACAN, elevated relative expression was observed for ACAN for all treatment groups. It is important to note that this response was acute, with days 14 and 28 returning to comparable levels to controls. Immunofluorescence (IF) staining of the cultures on day 7 confirms the relative expression of ACAN by Dec and Dxp. IL-1β also appears to induce morphologic changes as well as reduce ACAN staining. 100 μM of Dec may counteract the observed IL-1β responses. As demonstrated in FIG. 24, which is directed to day 28 relative expression of Col2a1, late phase changes were observed for Col2a1. Additionally, Lac treatment resulted in elevated Col2a1 relative expression and counteracts an observed reduction induced by Dxp alone.

In view of this example, low passage osteoarthritic chondrocytes exhibited: (1) an acute increase in the relative expression of ACAN with all the groups tested; (2) increased protein detected by IF staining caused by the increased transcription; (3) reduction in the detectable amount of ACAN by IF after 1 week in culture caused by the exposure of these cells to the proinflammatory cytokine, IL-1β; (4) protection against IL-1β-induced loss of ACAN staining; and (5) increase in the relative expression of Col2a1 and protection against Dxp-induced loss of Col2a1 transcription caused by extended exposure to Lac.

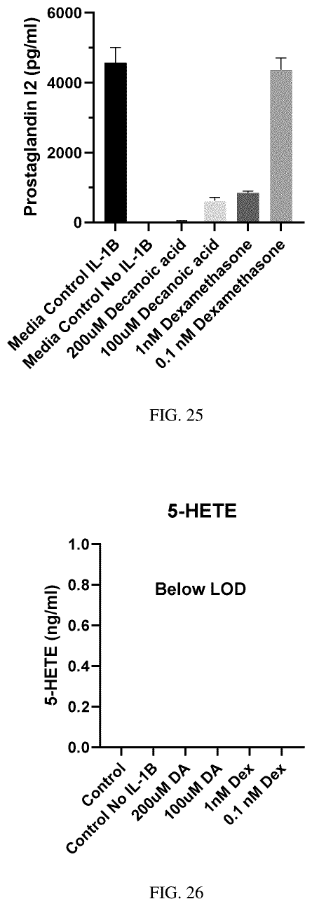

FIG. 25 is a bar graph that depicts the concentration of prostaglandin I₂ (PGI₂) present in primary human synoviocytes from patients with osteoarthritis that were stimulated with 1 ng/ml final concentration of IL-1β for 24 hours in the presence of varying concentrations of DA and Dex in view of controls. PGI₂ concentration response mirrored PGE₂ concentrations after DA treatment. IL-1β stimulation caused PGI₂ levels to increase in the cell culture media whereas DA treatment decreases secreted PGI₂ levels. It is noted that prostaglandin H₂ (PGH₂) was also tested and was not detected in either cell culture media or from cell lysates. Without being bound by theory, it is believed that PGH₂ is an intermediate and is (near) immediately converted into downstream prostaglandins and therefore levels of PGH₂ are not detectable.

Depicted in FIG. 26 is the concentration of 5(S)-Hydroxyeicosatetraenoic Acid (5-HETE) present in primary human synoviocytes from patients with osteoarthritis that were stimulated with 1 ng/ml final concentration of IL-1β for 24 hours in the presence of varying concentrations of DA and Dex in view of controls. No detectable levels of 5-HETE were found in synoviocytes.

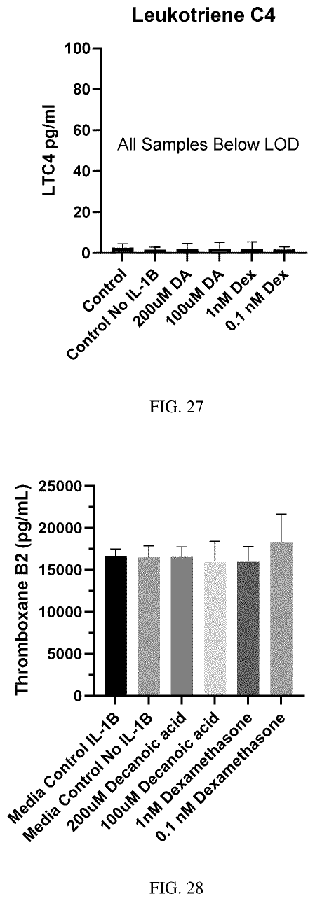

The concentration of leukotriene C₄ (LTC₄) present in primary human synoviocytes from patients with osteoarthritis that were stimulated with 1 ng/mL final concentration of IL-1β for 24 hours in the presence of varying concentrations of DA and Dex in view of controls is depicted in the bar graph of FIG. 27. All synoviocyte samples were below levels of detectability.

FIG. 28 depicts the concentration of thromboxane B2 (TBX2) present in primary human synoviocytes from patients with osteoarthritis that were stimulated with 1 ng/ml final concentration of IL-1β for 24 hours in the presence of varying concentrations of DA and Dex in view of controls. All samples resulted in similar concentrations of TBX2 (i.e., between about 15,000 and 20,000 pg/mL TBX2), with 0.1 nM of Dex resulting in the largest concentration of TBX2. No significant difference between any of the samples was observed.

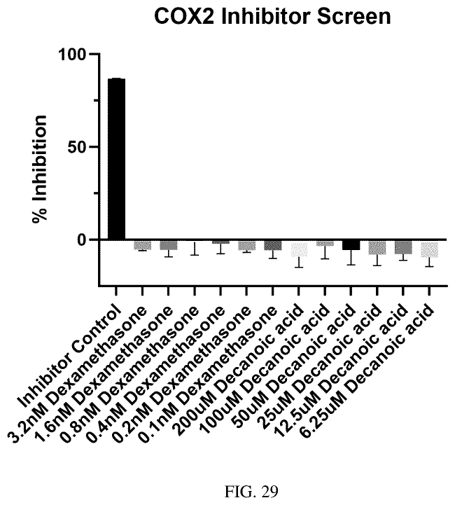

Depicted in FIG. 29 is an inhibitor screen for the enzyme Cyclooxygenase-2 (COX-2). Enzyme activity was monitored while in the presence of varying concentrations of DA and Dex. Celecoxib (Celebrex) was used as a positive control for COX-2. There was no observed inhibition of COX-2 activity with any of the concentrations of DA or Dex.

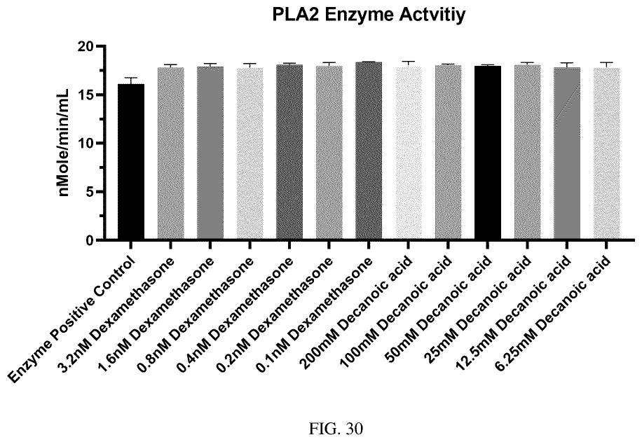

A phospholipase A2 (PLA2) activity assay using PLA₂ isolated from bee venom is depicted in FIG. 30. Positive control wells contained PLA₂ alone with substrate. The inhibitor test wells contained PLA2 along with substrate and varying concentrations of DA and Dex. All samples resulted in similar activities of PLA₂ (i.e., between about 15 and 20 nmole/min/mL) and no significant signs of PLA2 activity inhibition.

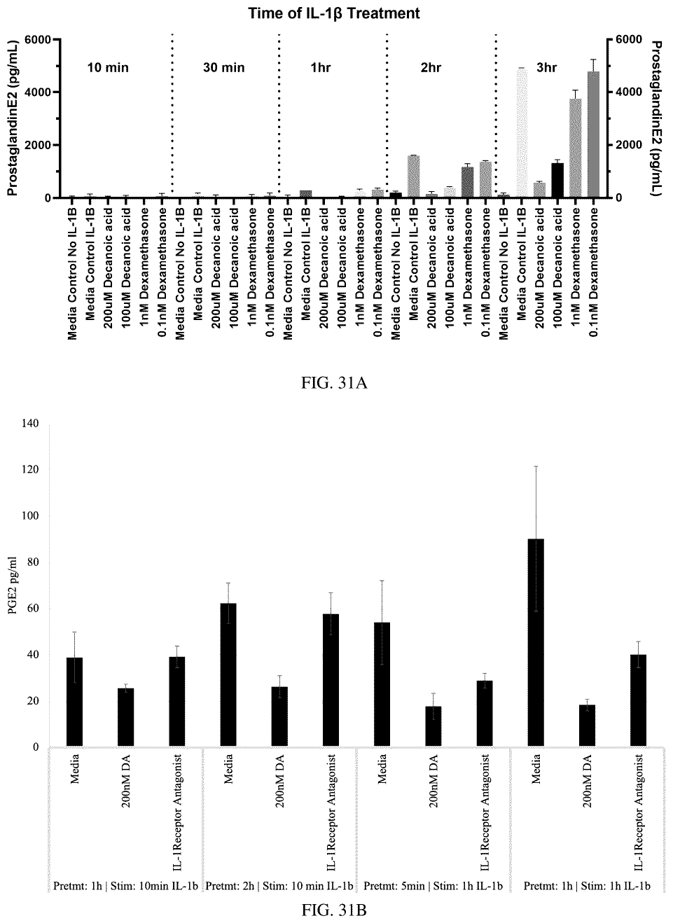

FIG. 31A depicts early concentrations of PGE₂ present in primary human synoviocytes from patients with osteoarthritis that were stimulated with 1 ng/mL final concentration of IL-1β for 24 hours in the presence of varying concentrations of Dex and DA in view of controls. The concentrations in FIG. 31A were measured at hour three. The samples with DA resulted in PGE₂ concentrations of less than about 5000 pg/ml, whereas the samples with Dex resulted in PGE₂ concentrations between about 5000 and 10,000 pg/ml.

FIG. 31B is a bar graph depicting PGE₂ release from human osteoarthritic synoviocytes treated with media, 200 nanomolar DA, or 500 nanograms/milliliter of IL-1 Receptor Antagonist (IL-1RA) for the specified pretreatment times at the bottom of each panel (5 minutes, 1 hour, or 2 hours, and then stimulated with 1 nanogram/milliliter IL-1β for either 10 minutes or 1 hour, as indicated. PGE₂ in picograms/milliliter was released at detectable levels in each of the media treated samples, including the short 10-minute IL-1β stimulation conditions, implicating low level activation caused by a change of media. The media controls within each panel of treatments released increased PGE₂ compared to DA treated cells. Cells were also treated with IL-IRA which inhibits IL-1β by binding the cell-surface IL-1 receptor accessory protein and inhibiting its dimerization with the IL-1 receptor (IL-1R), effectively blocking IL-1β signaling. IL-1β-mediated release of PGE₂ in IL-1RA treated cells was consistently higher than PGE₂ levels in DA treated cells. Further, IL-1RA treated cells showed similar levels of release compared to media controls after a 10-minute IL-1β stimulation and less than media after longer stimulation times, implying that the level of activation due to the change in media occurred in the IL-1RA treated cells as well. In contrast, DA-treatment blocked the low-level activation seen in media controls, implying that DA inhibits PGE₂ release prior to IL-1R signaling. DA is able to incorporate into the cell membrane_and the immediate inhibition of signaling may reflect the ability of DA to change the biophysical properties of the cell membrane.

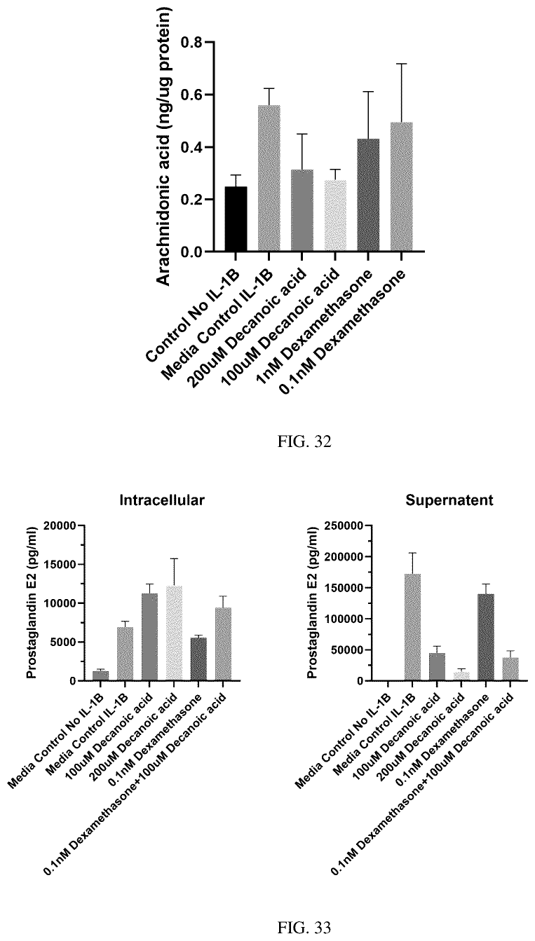

Early concentrations of arachidonic acid (AA) present in primary human synoviocytes from patients with osteoarthritis that were stimulated with 1 ng/mL final concentration interleukin 1-beta (β) for 3 hours in the presence of varying concentrations of Dex and DA in view of controls are depicted in FIG. 32. The concentrations of AA in FIG. 32 were normalized to total protein content from the cell lysates using bicinchoninic acid (BCA). Generally, the DA samples resulted in lower concentrations of AA than the Dex samples. All DA and Dex samples resulted in AA concentrations higher than that of the control with no IL-1β but lower than that of the control with IL-1β.

FIG. 33 illustrates two bar graphs that depict the concentrations of PGE₂ inside of the cell versus PGE₂ that was secreted into the cell culture media. Specifically, the bar graph labeled “Intracellular” illustrates the concentrations of PGE₂ inside of the cell and the bar graph labeled “Supernatent” illustrates the concentration of PGE₂ that was secreted into the cell culture media. Synoviocytes were stimulated with IL-1β for 6 hours and then cell culture media harvested separately along with collecting cell lysates. The data shows that PGE₂ concentration increases with IL-1β stimulation and that DA treatment causes PGE₂ to build up intracellularly instead of getting secreted. Specifically, treating osteoarthritic HSF cells with 1 ng/ml IL-1β final concentration causes the cells to secrete large amounts of PGE₂ into the cell culture media. As demonstrated in the previous examples and FIGS., treatment with DA causes PGE₂ secretion to be abrogated. To test this, the PGE₂ concentrations inside of the cells and in the cell culture media were measured to compare them directly to each other. The collected data shows an exact inverse trend when comparing the intracellular PGE₂ to the supernatant. This strongly suggests that DA is not affecting the production of PGE₂. Rather, the data suggest that DA is affecting the transport of DA outside of the cell and currently there is only one known transporter of PGE₂ that transports PGE₂ outside of the cell, the protein MRP4 (multidrug resistance protein 4). Thus, without being bound by theory, it is believed that DA binds to or interacts with MRP4 proteins and prevents MRP4 from successfully transporting PGE₂.

### Example 14. Analysis of Dex and DA in Treating Monoiodoacetate (MIA)-Induced Osteoarthritis in a Rat Model Animal Study

MIA is a model of inflammation of the knee joint used to evaluate the inflammatory component of osteoarthritis. The MIA model has become a standard for modeling joint disruption in osteoarthritis in rodents and involves injection of MIA into a knee joint that induces rapid pain-like responses in the ipsilateral limb, the level of which can be controlled by injection of different doses. The objective of this Example 14 was to evaluate the efficacy of several compounds on behavioral, functional, inflammatory and structural effects in response to chemically induced inflammatory osteoarthritis at various time points post injury and at 28 days post injury. The results of this Example 14 are not powered as estimates of effect size and variability were unknown.

In this Example, 35 male Spraque-Dawley rats were enrolled. Each rat was randomly assigned to one of seven groups based on baseline body weight, the von Frey test, and weight bearing test results. Each group had five rats. Group 1 was the sham group and groups 2 to 7 were the model groups. The sham group (Group 1) received intraarticular (IA) saline and all other groups received 2 mg MIA IA (a moderate dose) to induce inflammation. Groups 2 to 7 were than assigned to a treatment of saline, LR, DA or sodium decanoate in LR, Dex phosphate in LR, Ins101 in saline, or Ins101 in LR, where Ins101 is a combination of DA and Dex. IA injections were then administered twice a week for a total of 6 injections.

Specifically, the treatment compounds were as follows: (1) a saline treatment that included 0.9% NaCl; (2) an LR treatment that included 0.336 mg/kg dose or 28 mM LR solution; (3) a DA in LR treatment that included 0.006 mg/kg DA dose or 250 μM DA solution in LR; (4) a Dex phosphate in LR treatment that included 0.01 mg/kg Dex dose or 150 μM Dex solution in LR; (5) an Ins101 in saline treatment that included 250 μM DA+150 μM Dex-P in saline; and (6) an Ins101 in LR treatment that included 250 μM DA+150 μM Dex-P in LR.

By way of dosage motivation, typical Dex phosphate injections to the knee of a human are about 4 mg, which calculates to about 0.057 mg/kg for a 70 kg person. This Example 14 used an animal dose of about 0.01 mg/kg, or approximately 5.5 times less than the average human dose. The groups were as follows: Group 1—Sham (Saline, 30 μL, 6 injections, N=5), Group 2—Model (Saline, 30 μL, 6 injections, N=5), Group 3—Model (LR, 30 μL, 6 injections, N=5), Group 4—Model (DA in LR, 30 μL, 6 injections, N=5), Group 5—Model (Dex in LR, 30 μL, 6 injections, N=5), Group 6—Model (DA+Dex in saline, 30 μL, 6 injections, N=5), and Group 7—Model (DA+Dex in LR, 30 μL, 6 injections, N=5).

To begin, the paw withdrawal threshold (PWT) weight was taken and von Frey and weight bearing tests were conducted for each rat to determine their baseline. Each rat was then injected with saline or MIA in the right hind leg, group assignments were established, and the rats were dosed according to their group assignment twice a week for up to 6 dosings. Specifically, the first dosing was administered on day 3 and the last dosing was administered on day 21. PWT, von Frey, and weight bearing was determined at least again on day 3 and on day 27 in addition to the baseline determination. On day 28, serum was collected from the animals and the knees were collected, specifically, the right treatment knee and the left untreated knee.

Markers analyzed in this Example were general health indicators (i.e., body weight), pain indicators (i.e., von Frey and weight bearing), inflammatory markers (i.e., pro-inflammatory cytokines including Tumor Necrosis Factor-Alpha (TNF-α), IL-1β, and interleukin-6 (IL-6)), markers of cartilage degradation (i.e., proteases that degrade collagen including MMP3 and MMP13, and cartilage degradation including CTX-II and COMP), structural assessment (i.e., μCT scanning), and histology (i.e., degradation level of articular cartilage via mankin score and assessment of synovitis via synovial injury score.

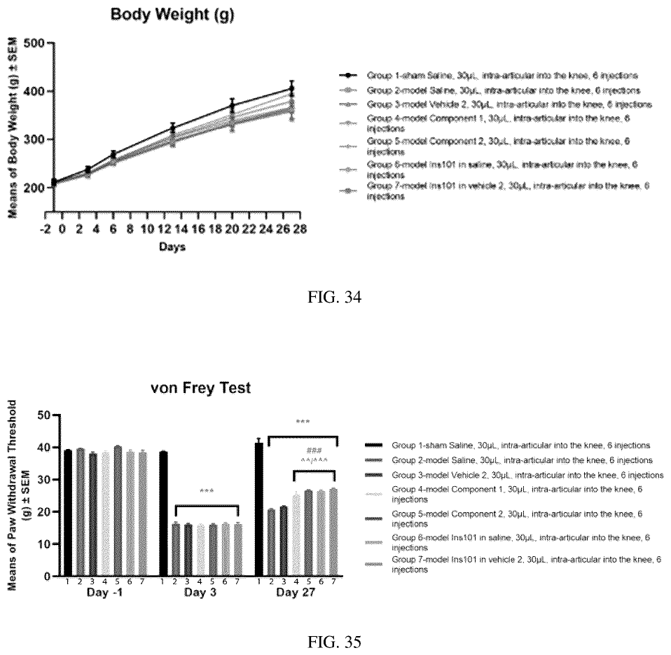

With regard to body weight (i.e., PWT weight), which was tracked throughout the study, the rats were allowed to feed freely and were expected to gain weight when healthy. That is, poor weight gain was taken as an indication of illness or reduced mobility. FIG. 34 depicts the body weight for each group throughout the study. As can be seen in FIG. 34, the body weight of all rats increased steadily throughout the study and no statistically significant difference in body weight was found among all groups.

The mechanical sensitivity (von Frey) test includes applying a thin filament to the plantar surface of the hind paw at increasing force to elicit a paw withdrawal. The mechanical withdrawal threshold is defined as force that elicits a withdrawal reflex measured in grams. The purpose of this test is to measure mechanical nociception to evaluate the ability of an animal to detect a noxious stimulus. FIG. 35 depicts the results of the von Frey test for each group taken as the baseline (i.e., day −1) and on day 3 and day 27. The treatment injections associated with Groups 4, 5, 6, and 7 (e.g., DA in LR, Dex in LR, Ins101 in saline, and Ins101 in LR, respectively) exhibited better efficacy in decreasing mechanical hyperalgesia than that of the Saline or LR injections associated with Groups 2 and 3.

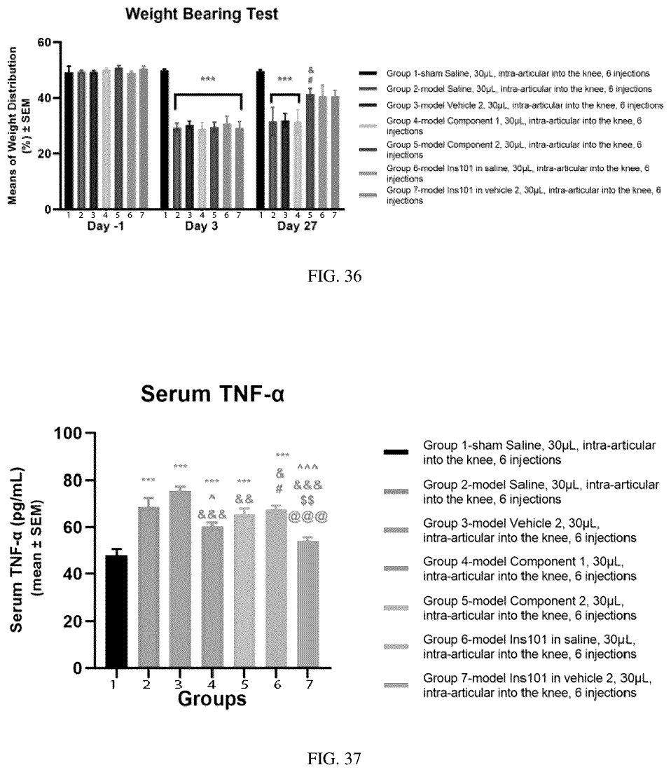

Weight bearing can be used as an indication of functional pain. To test weight bearing, Hind legs are placed on scale measuring weight bearing of both left and right hind limbs simultaneously. Weight-bearing percentage of the right hind limb is calculated, as the MIA is injected into the right hind leg. FIG. 36 depicts the results of the weight bearing test for each group taken as the baseline (i.e., day −1) and on day 3 and day 27. The treatment injections associated with Groups 5, 6, and 7 (e.g., Dex in LR, Ins101 in saline, and Ins101 in LR, respectively) resulted in greater means of weight distributions than that associated with Groups 2, 3, and 4. Additionally, no statistically significant difference in the weight distribution of Groups 5, 6, and 7 was found when compared with the sham group (i.e., Group 1), suggesting the efficacy of Dex, Ins101 in saline, and Ins101 in LR.

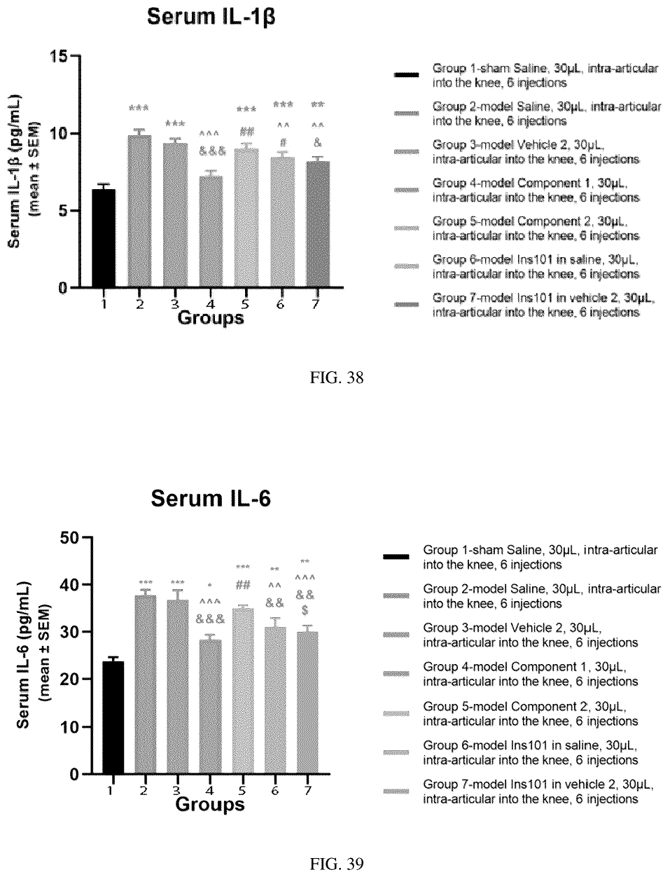

Inflammation markers, such as TNF-α, IL-1β, IL-6, were measured in the serum of each rat at the end of the study. TNF-α, IL-1β, IL-6 are potent pro-inflammatory cytokines that are implicated in the pathogenesis of osteoarthritis and are differentially regulated. FIG. 37 depicts TNF-α measured in the serum for each rat and then grouped accordingly. The concentrations were measured at the end of a 28-day MIA-induced Osteoarthritis animal study analyzing the effect of Dex and DA, and combinations thereof in treating inflammation of a knee joint. Groups 4 and 7 administered with DA in LR and Ins101 in LR, respectively were statistically significantly lower than those of Group 2 administered with saline, suggesting the efficacy of DA and Ins101 in LR. FIG. 38 depicts IL-1β measured in the serum for each rat and then grouped accordingly. The concentrations were measured at the end of a 28-day MIA-induced Osteoarthritis animal study analyzing the effect of Dex and DA, and combinations thereof in treating inflammation of a knee joint. Groups 4, 6, and 7 administered with DA in LR, Ins101 in saline, and Ins101 in LR, respectively resulted in statistically significantly lower IL-1β than Group 2 administered with saline, suggesting the efficacy of DA, Ins101 in saline and in LR. Only for Group 4 (DA in LR) was no statistically significant difference observed compared with Group 1 (sham), suggesting that DA was better than Ins101 in saline or in LR at inhibiting inflammation. FIG. 39 depicts IL-6 measured in the serum for each rat and then grouped accordingly. The concentrations were measured at the end of a 28-day MIA-induced Osteoarthritis animal study analyzing the effect of Dex and DA, and combinations thereof in treating inflammation of a knee joint. Groups 4, 6, and 7 administered with DA in LR, Ins101 in saline, and Ins101 in LR, respectively resulted in statistically significantly lower IL-6 than Group 2 administered with saline, suggesting the efficacy of DA, Ins101 in saline and in LR.

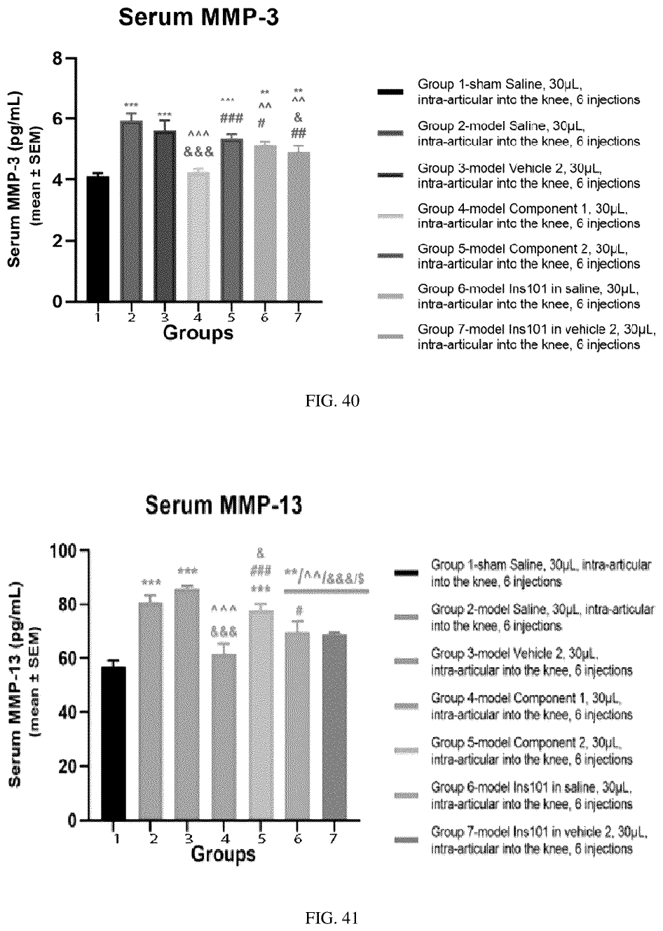

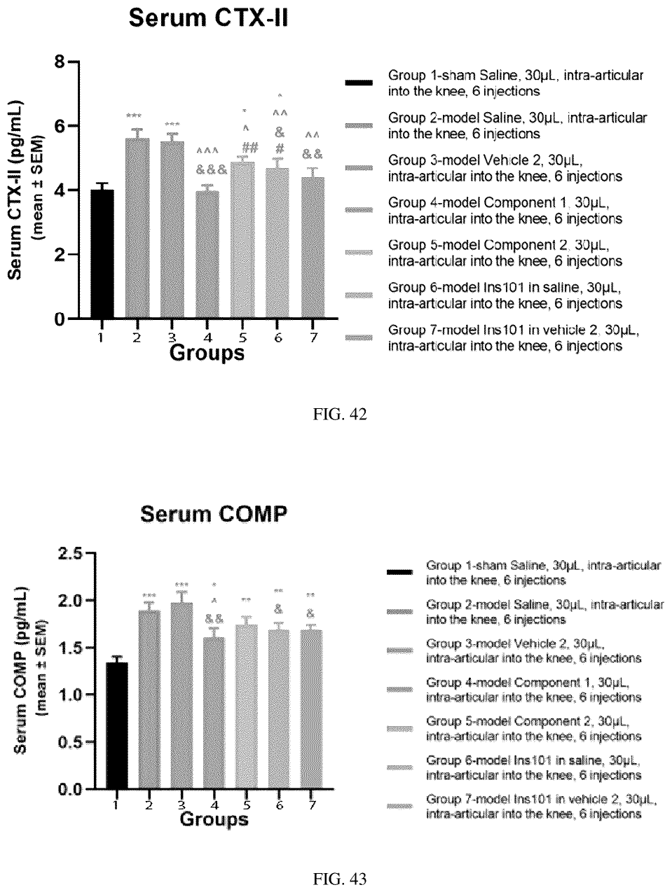

Markers of cartilage degradation used in this study include MMP3, MMP13, cartilage oligomeric matrix protein (COMP), and C-terminal telopeptide of type II collagen (CTX-II). MMP3 degrades collagen types II, III, IV, IX, X and proteoglycans and can activate other MMP's. MMP13 primarily degrades collagen type II (primary component of healthy cartilage). COMP is a non-collagenous extracellular matrix protein that increases in serum as a marker of cartilage turnover and degradation. CTX-II is degradation product of type II collagen, which is released into the serum and urine, serving as a marker of cartilage breakdown. FIG. 40 depicts MMP3 measured in the serum for each rat and then grouped accordingly. The concentrations were measured at the end of a 28-day MIA-induced Osteoarthritis animal study analyzing the effect of Dex and DA, and combinations thereof in treating inflammation of a knee joint. Groups 4, 6, and 7 administered with DA in LR, Ins101 in saline, and Ins101 in LR, respectively resulted in statistically significantly lower MMP3 than Group 2 administered with saline, suggesting the efficacy of DA, Ins101 in saline and in LR. Only for Group 4 (DA in LR) was no statistically significant difference observed compared with Group 1 (sham), suggesting that DA was better than Ins101 in saline or in LR at inhibiting inflammation. FIG. 41 depicts MMP13 measured in the serum for each rat and then grouped accordingly. The concentrations were measured at the end of a 28-day MIA-induced Osteoarthritis animal study analyzing the effect of Dex and DA, and combinations thereof in treating inflammation of a knee joint. Groups 4, 6, and 7 administered with DA in LR, Ins101 in saline, and Ins101 in LR, respectively resulted in statistically significantly lower MMP13 than Group 2 (administered with saline) and Group 3 (administered with LR), suggesting the efficacy of DA, Ins101 in saline and in LR. Only for Group 4 (DA in LR) was no statistically significant difference observed compared with Group 1 (sham), suggesting that DA was better than Ins101 in saline or in LR at inhibiting inflammation. FIG. 42 depicts CTX-II measured in the serum for each rat and then grouped accordingly. The concentrations were measured at the end of a 28-day MIA-induced Osteoarthritis animal study analyzing the effect of Dex and DA, and combinations thereof in treating inflammation of a knee joint. Groups 4, 5, 6, and 7 administered with DA in LR, Dex in LR, Ins101 in saline, and Ins101 in LR, respectively resulted in statistically significantly lower CTX-II than Group 2 (administered with saline), suggesting the efficacy of DA, Dex, Ins101 in saline and in LR. The absolute results indicate that DA was better than Dex and Ins101 in saline at inhibiting inflammation.

FIG. 43 depicts COMP measured in the serum for each rat and then grouped accordingly. The concentrations were measured at the end of a 28-day MIA-induced Osteoarthritis animal study analyzing the effect of Dex and DA, and combinations thereof in treating inflammation of a knee joint. Group 4 (administered with DA in LR) resulted in statistically significantly lower COMP than Group 2 (administered with saline), suggesting the efficacy of DA.

In FIGS. 37-43, * denotes statistical significance in view of Group 1, a sham group. \hat{ } denotes statistical significance in view of Group 2, a saline control group. & denotes statistical significance in view of Group 3, an LR control group. # denotes statistical significance in view of Group 4, a DA in LR group. $ denotes statistical significance in view of Group 5, a Dex in LR group. @ denotes statistical significance in view of Group 6, a Dex plus DA in LR group.

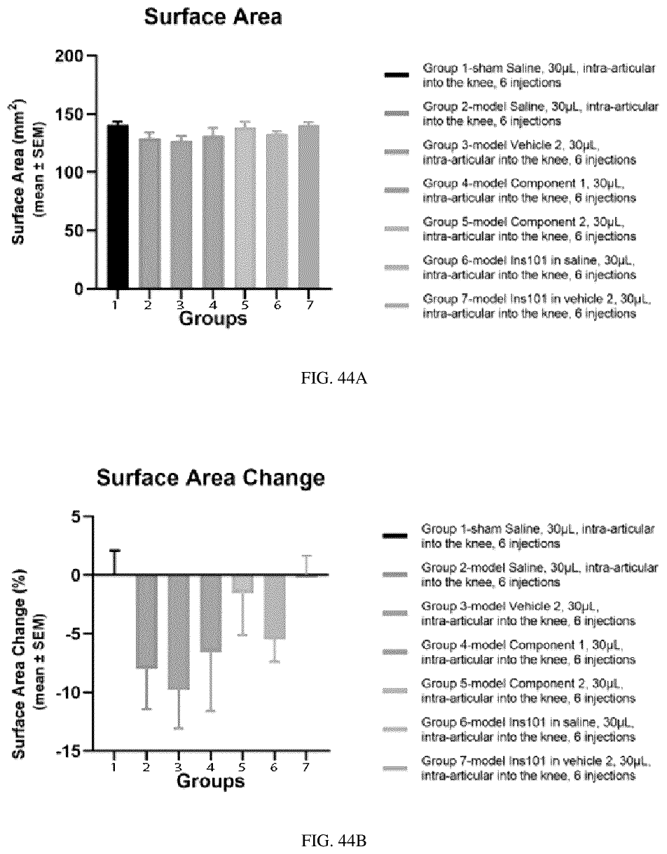

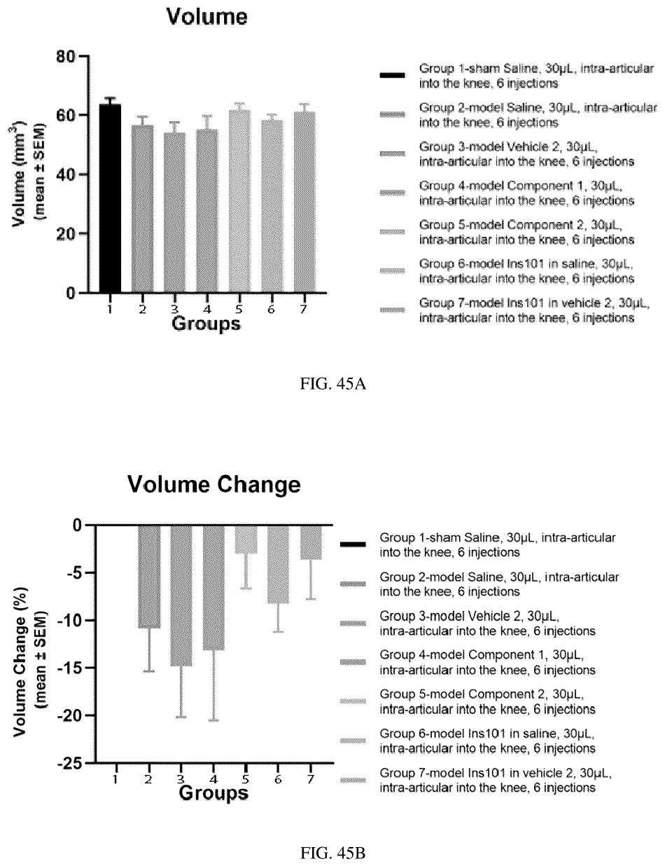

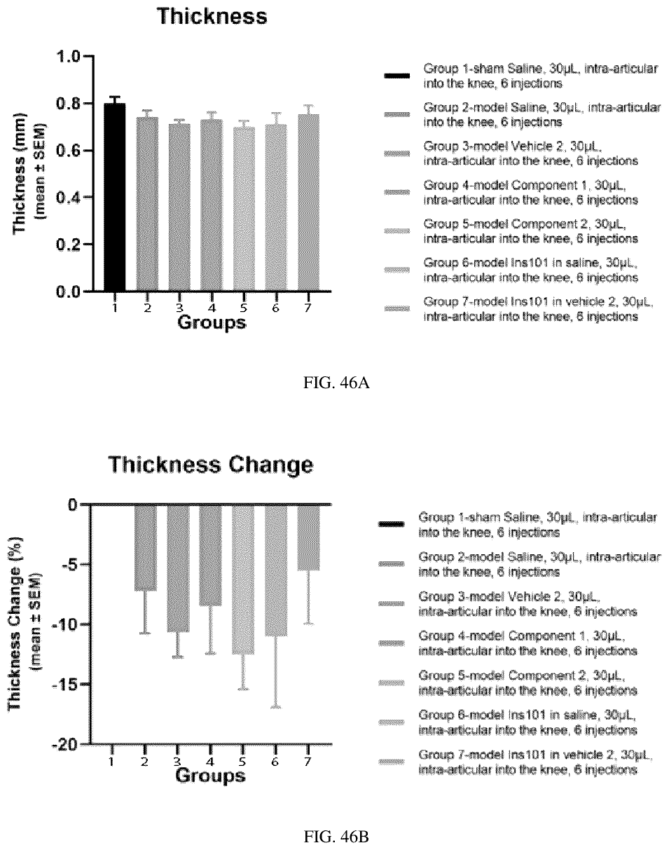

The joints of the rats were also assessed. Knees were fixed post-mortem, subjected to μCT scanning and 3-D images were generated to determine: articular cartilage surface area, articular cartilage volume, articular cartilage thickness. FIGS. 44A and 44B depict articular cartilage surface area and surface area change, respectively, calculated for each rat and grouped accordingly. The surface area and surface area change were determined via μCT scanning and generated 3D images at the end of a 28-day MIA-induced Osteoarthritis animal study analyzing the effect of Dex and DA, and combinations thereof in treating inflammation of a knee joint. No statistical significance was observed between the surface areas calculated for each group. FIGS. 45A and 45B depict articular cartilage volume and volume change, respectively, calculated for each rat and grouped accordingly. The volume and volume change were determined via μCT scanning and generated 3D images at the end of a 28-day MIA-induced Osteoarthritis animal study analyzing the effect of Dex and DA, and combinations thereof in treating inflammation of a knee joint. No statistical significance was observed between the volumes calculated for each group. FIGS. 46A and 46B depict articular cartilage thickness and thickness change, respectively, calculated for each rat and grouped accordingly. The thickness and thickness change were determined via μCT scanning and generated 3D images at the end of a 28-day MIA-induced Osteoarthritis animal study analyzing the effect of Dex and DA, and combinations thereof in treating inflammation of a knee joint. No statistical significance was observed between the volumes calculated for each group.

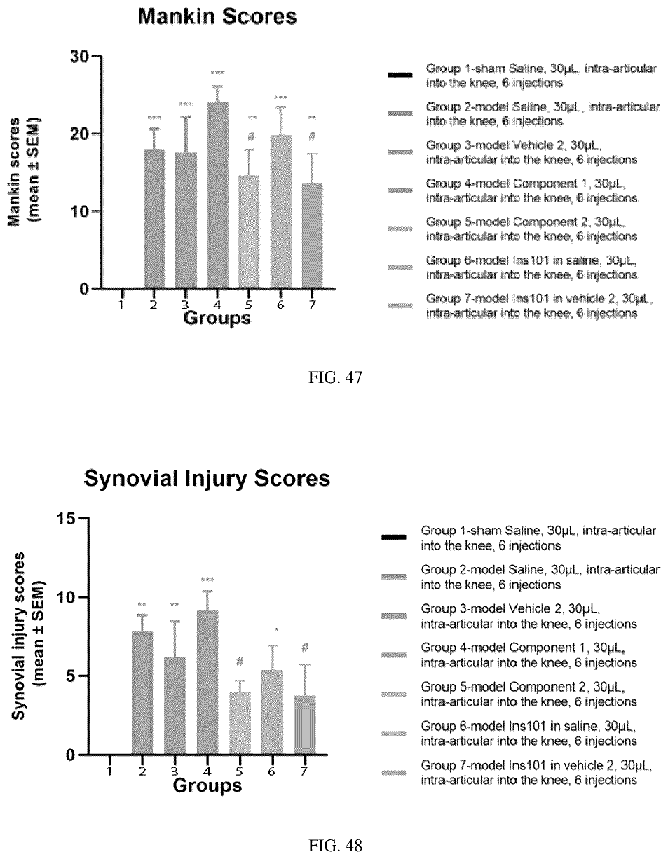

Mankin scores investigate the degradation level of the articular cartilage by assessing four parameters including: cartilage structure, cellularity, proteoglycan depletion and tidemark integrity. Each parameter has subcategories, and the scores are summed to provide a total score ranging from 0 (normal) to 14 (most severe osteoarthritis). FIG. 47 depicts the Mankin scores determined for each rat and then grouped accordingly. Mankin scores indicate the degradation level of articular cartilage by assessing four parameters including: cartilage structure, cellularity, proteoglycan depletion, and tidemark integrity and were calculated at the end of a 28-day MIA-induced Osteoarthritis animal study analyzing the effect of Dex and DA, and combinations thereof in treating inflammation of a knee joint. Groups 5 (Dex in LR) and 7 (Ins101 in LR) were statistically significantly lower than those of Group 4 (DA in LR), suggesting that the efficacy of Dex in LR and Ins101 in LR was better than that of DA.

Synovial injury scores are based on three components of synovitis: lining layer hyperplasia, activation of resident cells (stroma), and inflammatory infiltrate. Each of these components are graded semi-quantitatively from 0 to 3 and the total score ranges from 0 to 9 to calculate the synovial injury score. High-grade synovitis is defined by a score higher than 4. FIG. 48 depicts the synovial injury scores determined for each rat and then grouped accordingly. The synovial injury scores are based on three components of synovitis: lining layer hyperplasia, activation of resident cells (stroma) and inflammatory infiltrate and were calculated at the end of a 28-day MIA-induced Osteoarthritis animal study analyzing the effect of Dex and DA, and combinations thereof in treating inflammation of a knee joint. Groups 5 (Dex in LR) and 7 (Ins101 in LR) were statistically significantly lower than those of Group 4 (DA in LR), suggesting that the efficacy of Dex in LR and Ins101 in LR was better than that of DA.

TABLE 3
Efficacy Summary versus Controls.

| | | | Combination |
|---|---|---|---|
| Test | Dex in LR | DA in LR | (Ins101) in LR |
| von Frey | + | + | ++ |
| Weight Bearing | + | − | + |
| TNF-α | − | + | ++ |
| IL-1β | − | ++ | + |
| IL-6 | − | + | + |
| MMP-3 | − | ++ | + |
| MMP-13 | − | ++ | + |
| CTX-II | +/− | + | + |
| COMP | +/− | + | + |
| Surface Area | − | − | +/− |
| Volume | − | − | − |
| Thickness | − | − | − |
| Mankin | + | − | + |
| Synovial Injury | + | − | + |

The study of Example 14 was conducted to evaluate the in vivo efficacy of the test articles in the monosodium iodoacetate-induced arthritis (MIA) model in rats. As scheduled, the rats of Group 1 were set as sham controls, and the rats of Groups 2-7 were established as MIA models and administered with different treatments.

At the end of the study, the paw withdrawal threshold (PWT), and weight bearing test results of Group 2 (model group administered with “Saline”) were statistically significantly lower than those of Group 1. Besides, the biomarkers levels (including MMP3, MMP13, CTX-II, TNF-α, IL-1β, IL-6, and COMP) and histological scores (Mankin scores and synovial injury scores) of Group 2 were statistically significantly higher than those of Group 1. These results indicated the successful establishment of the MIA model.

According to the behavioral results, the test articles, DA, Dex, Ins101 in saline, and Ins101 in LR, exhibited efficacy in decreasing mechanical hyperalgesia. The test articles, Dex, Ins101 in saline, and Ins101 LR, seemed to significantly improve the weight distribution.

According to the serum biomarkers levels, the rats which were administered with test articles DA, Ins101 in saline, and Ins101 in LR exhibited lower MMP3, MMP13, CTX-II, TNF-α, IL-1β, IL-6, and COMP levels when compared with Group 2 which was administered with “Saline”. The rats which were administered with DA has significantly lower levels of MMP3, MMP13, CTX-II, and IL-1β as no statistically significant difference in MMP3, MMP13, CTX-II, and IL-1β levels was found between Group 4 and Group 1.

According to the histological scores, the Mankin scores and synovial injury scores were lower in the rats which were administered with Dex and Ins101 in LR when compared with other groups.

Above all, the test articles DA, Dex, Ins101 in saline, and Ins101 in LR could improve arthritis symptoms at varying degrees.

### Example 15. Regulation of Chondrocytes Via SOX9 and RUNX2 Transcription

Chondrocytes are essential for the overall health of articular joints because they help maintain an equilibrium between anabolic and catabolic substances produced in the microenvironment. Two master regulatory transcription factors that aid in this process are SOX9 and RUNX2. SOX9 secures proper chondrocyte lineage commitment, promotes cell survival, and regulates cartilage-specific structural components such as type II Col2a1 and ACAN. Conversely, RUNX2 drives the expression genes for cartilage degradation, hypertrophic differentiation, and ossification, such as MMP13. Furthermore, overexpression of SOX9 has been shown to ameliorate the course of experimental osteoarthritis, whereas RUNX2 overexpression causes post-traumatic osteoarthritis progression.

To evaluate how compounds of interest impact cellular plasticity, human osteoarthritic chondrocytes (passage 5) were cultured for 27 days in the presence of either 50 micromolar decanoate (Dec), 0.1 micromolar Dxp, 100 millimolar D/L Lac, Dec+Dxp, or Dec+Dxp+Lac. The culture mediums were replenished weekly by withdrawing about half of the volume and replacing them with warmed mediums supplemented as needed. After 27 days, total RNA was extracted and qRT-PCR was carried out with primer sets for SOX9, RUNX2, and GAPDH.

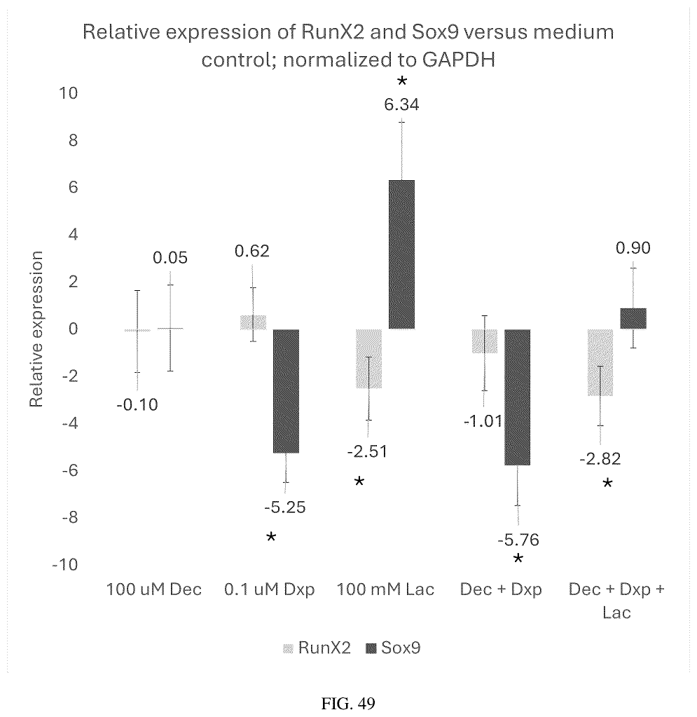

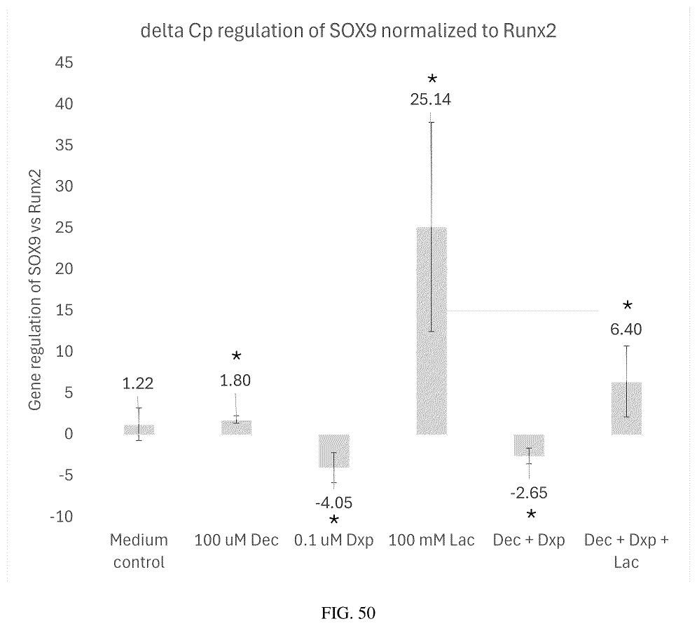

Relative expression analysis reveals that Dxp treatment of these cells reduces the detectable amount of SOX9 messenger RNA, whereas treatment with Lac enhances SOX9 and decreases RUNX2. When delta-delta relative gene expression versus medium controls is determined, with normalization to GAPDH, −5.25±1.23 and −5.76±1.70-fold-reductions in SOX9 for the Dxp and Dec+Dxp treatment groups, respectively, were observed. Conversely, Lac treatment increased SOX9 by 6.34±2.46-fold. Furthermore, the combination of Dec+Dxp+Lac demonstrated a relative expression of 0.90±1.69 for SOX9. Evaluation of RUNX2, on the other hand, demonstrated that Lac and Dec+Dxp+Lac exhibited significant reductions in RUNX2 relative expressions of −2.51±1.33 and −2.872±1.26, respectively. Moreover, when the expression of SOX9 was compared to RUNX2 in these samples, it was discovered that Dec, Lac, and Dec+Dxp+Lac increased the amount of SOX9 in relation to RUNX2 (1.80±0.47, 25.14±12.71, and 6.40±4.51 respectively), while Dxp and Dec+Dxp decreased levels (−4.05±1.81 and −2.65±0.96 respectively). These data suggest that Lac treatment of these cells preserves beneficial chondrocyte lineages and protects against Dxp-induced reductions in SOX9 expression. FIG. 49 depicts the relative expression of RUNX2 and SOX9 in low passage human osteoarthritic chondrocytes treated with 100 μM decanoate (Dec), 0.1 μM Dxp, 100 mM d/l Lac, and combinations (n=4; * p-value=>0.05 one sample, two-tailed t-test versus hypothetical 1). FIG. 50 depicts the relative expression of SOX9 normalized to RUNX2 in low passage human osteoarthritic chondrocytes treated with 100 μM decanoate (Dec), 0.1 μM Dxp, 100 mM d/l Lac, and combinations (n=4; * p-value=>0.05 one sample, two-tailed t-test versus hypothetical 1).

### Example 16. Regulation of Chondrocyte Col1a1 Transcription

Evidence suggests that SOX9 and RUNX2 levels are essential for converting chondrocytes into osteoblasts. Osteoblasts are bone-forming, and their dysregulation has been associated with the development of osteoarthritis. Without being bound by theory, it is believed that SOX9 prevents chondrocytes from dedifferentiating into skeletogenic precursor cells that can then transdifferentiate into osteoblasts. In contrast, RUNX2 enhances the survival of hypertrophic chondrocytes, which can differentiate into osteoblasts. Furthermore, Col1a1 is regarded as both a marker of osteoblast lineage and fibrosis.

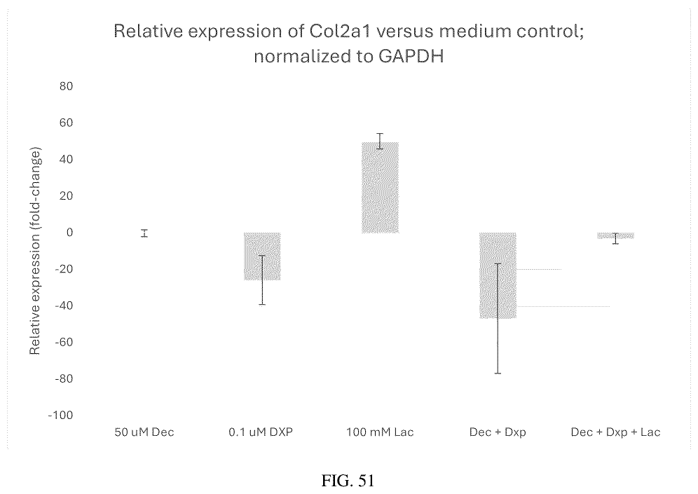

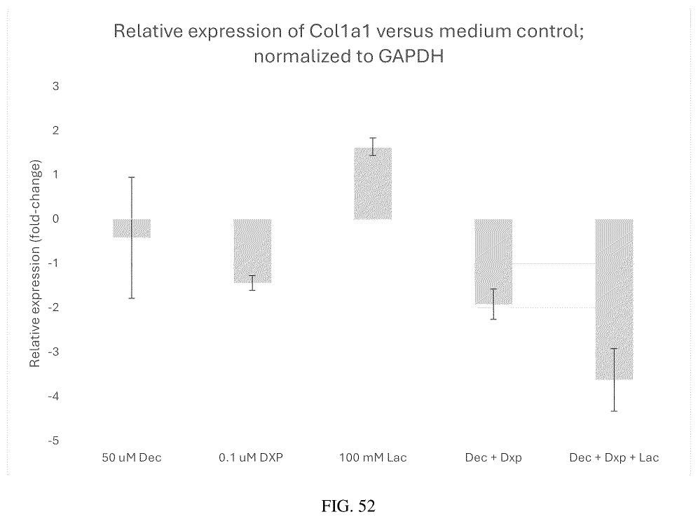

To evaluate the biological ramifications of the SOX9 to RUNX2 regulation mentioned above, delta-delta relative gene expression was assessed against medium controls, with normalization to GAPDH for both Col2a1 and Col1a1 in the samples tested with reference to Example 15. It was observed that a 27-day treatment of these chondrocytes with 0.1 μM Dxp resulted in a global reduction in collagen regulation, noted as a −25.99±13.34-fold reduction in Col2a1 and a −1.44±0.17-fold reduction in Col1a1. It is important to highlight that a similar response was observed in the combined Dec+Dxp treatment group. Moreover, treatment with 100 mM Lac increased collagen expression (49.88±4.31 and 1.63±0.20 for Col2a1 and Col1a1, respectively; FIGS. 51 and 52).

Unexpectedly, when Dec, Dxp, and Lac treatments were combined, no significant change in Col2a1 regulation was observed (−3.24±2.91-fold) but an enhanced reduction in Col1a1 was (−3.63±0.70). Taken together, without being bound by theory, it is hypothesized that the skewed SOX9 to RUNX2 ratios achieved in the treatment groups are driving unique phenotypes of chondrocytes. In particular, the Dec+Dxp+Lac group may be preventing osteoblast development, as indicated by elevated ratios of Col2a1 to Col1a1. Furthermore, the presence of Lac prevents reduced Dxp-induced reductions in Col2a1.

### Example 17. Upregulation of ACAN Transcription by Lac

One of the hallmarks of osteoarthritis is an early and profound loss of ACAN, a proteoglycan decorated with glycosaminoglycan side chains that binds to hyaluronan and other proteins to form hydrated gel structures, allowing cartilage to endure compressive pressures. Proteolytic enzymes specific to ACAN are found in arthritic cartilage and are responsible for its breakdown, which is thought to contribute to pathogenesis. Common investigation modalities against osteoarthritis include measures to inhibit ACAN degradation and/or restore its production.

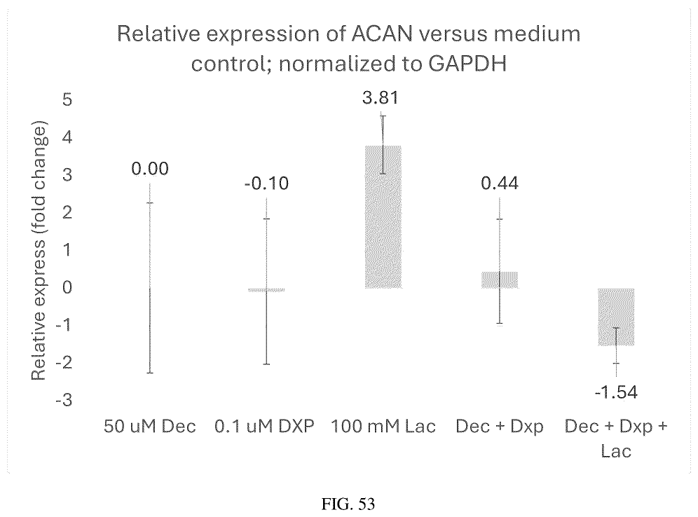

Delta-delta relative gene expression was employed to determine ACAN regulation in the 27-day samples referenced in Examples 15 and 16. A 3.81±0.77-fold increase in ACAN expression in the cells after 27 days of Lac treatment was observed. Unexpectedly, a slight reduction in ACAN in the Dec+Dxp+Lac treatment group (−1.54±0.47) was also observed. Without being bound by theory, it is hypothesized that these findings indicate that metabolic management and/or metabolite feedback loops represent promising therapy targets for osteoarthritis. Results associated with Example 17 are depicted in FIG. 53.

### Example 18. Consideration of Additional Drug Compounds of Interest for Disease Modalities of the Present Disclosure

Standardized models of the present disclosure were also employed to identify other compounds of interest. It was hypothesized that two additional compounds should be investigated to determine if they have the potential for synergistic and/or positive effects in treating osteoarthritis alongside the compounds disclosed herein. The first was alpha-lipoic acid (LA), a natural antioxidant known to reduce inflammation, NF-κB activation, and oxidative stress. The second was tryptophan (Try), an amino acid that degrades into key metabolites that help regulate the immune system.

These compounds were tested in a high passage osteoarthritic chondrocyte monolayer model, alone or in combination with other agents, for both transcriptional regulation of standard markers and IL-1β-induced PGE₂ production. It was observed that LA was effective in both transcriptional remodeling and acute inflammation in dedifferentiated osteoarthritic chondrocytes. For example, treatment with 100 mm LA in combination with 28 mM Lac resulted in the possible synergy of Col2a1 and SOX9 in these cells in the presence of TGF-β. When delta-delta relative expression with GAPDH was assessed after 14 days in culture, a 30-fold, 218-fold, and 1618-fold increase in Col2a1 was observed compared to controls for Lac alone, LA alone, and Lac+LA. SOX9 transcription increased 2-fold, 4-fold, and 9-fold, respectively. Furthermore, LA treatment inhibited IL-1β-induced PGE₂ release from these cells after 24 hours in a dose-dependent manner (93%, 44%, and 33% at final concentrations of 100, 10, and 1 μM, respectively). When Try was combined with DA, transcriptional analysis of these models suggested possible synergistic effects as well. After 28 days of treatment, MMP13 regulation decreased by −3.32-, 1.12-, and −321-fold for 1 mM Try, 0.5 mM DA, and Try+DA, respectively. Conversely, when TGF-β was added to the model, Col2a1 was increased by −1.36-, 26-, and 195-fold for Try, DA, and DA+Try.

### Example 19. Rescue Versus Preincubation Treatment Regimens for IL-1β-Induced PGE₂ Release Response

The mechanisms of action (MOA) of a drug often dictate the timing and delivery of treatment regimens. While the MOA of compounds disclosed herein continue to be elucidated, experiments were designed to help establish how the drugs should be administered. Furthermore, these experiments will also help with hypothesis generation for subsequent MOA investigations. A low-passage chondrocyte monolayer model was utilized to establish how changes in drug delivery impact the inhibition of IL-1β-induced PGE₂ release seen following treatment with the compounds under investigation.

Passage 3 normal human chondrocytes were seeded into tissue culture plates and then tested for compound activity by pre-treating for 24 hours, administering at the same time as activation, or by adding to cells following an overnight activation with IL-1β. Treatment groups included medium control, 25 μM decanoate (Dec), 28 mM D/L sodium Lac, and 10 μM LA. PGE₂ release was then evaluated 24- or 96-hours post-activation with percent inhibitions calculated versus plate- and block-matched controls. The experiments were performed in singlet and are intended to be repeated to establish variance.

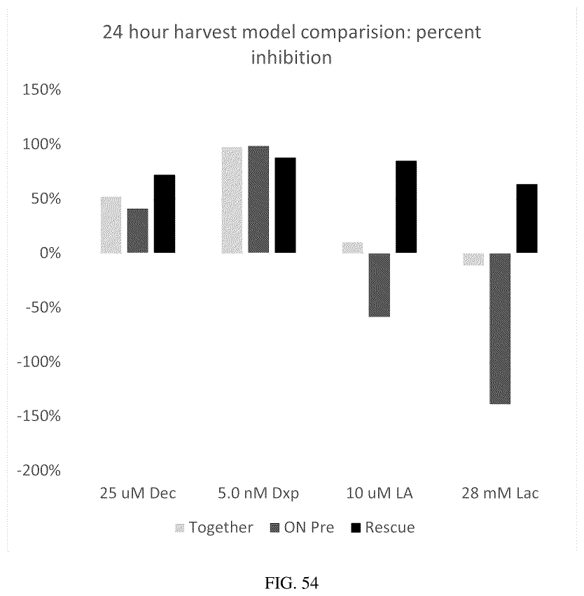

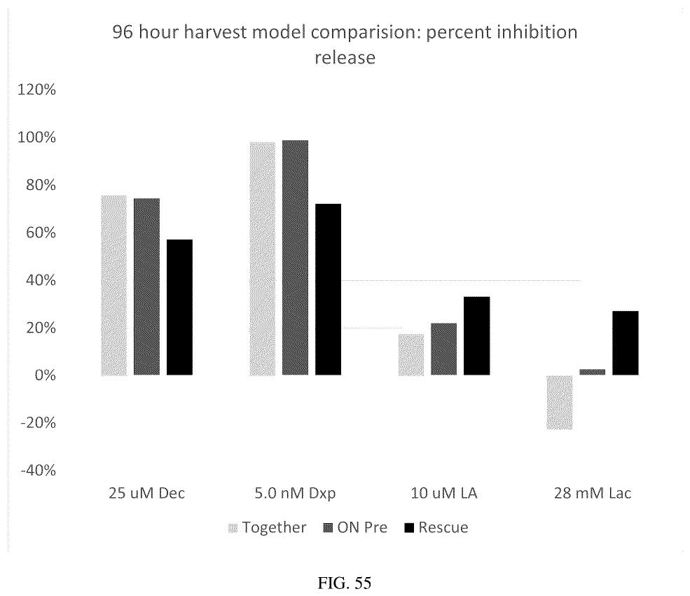

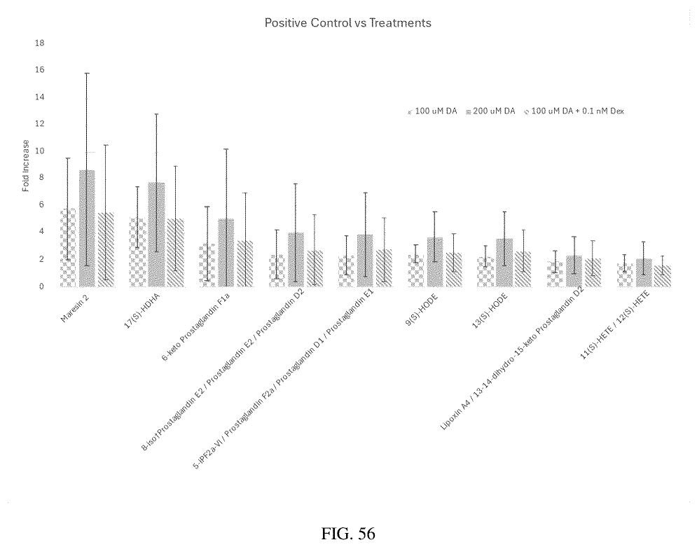

It was observed that pre-incubating the cells overnight with Dec before activation and adding at the same time as IL-1β resulted in similar inhibitions in PGE₂ release in the 24-hour or 96-hour harvest models. However, greater overall inhibitions of PGE₂ release were observed with Dec treatment in the 96-hour harvest model. Dec treatment also successfully rescued IL-1β-activated chondrocytes from excessive PGE₂ release. Interestingly, the rescue resulted in better inhibitions in the 24-hour harvest model than adding compounds together or following a pre-incubation (72% vs 53% or 41%). In the 96-hour harvest model, however, the rescue was lower than adding together or with pre-incubation (57% vs 76% or 75%). Moreover, pre-incubation with LA or Lac may potentiate the PGE₂ release in the 24-hour model with −59 and −139% inhibitions observed. Conversely, LA or Lac reduced the detectable amount of PGE₂ in the rescue models by 85% and 64% in the 24-hour harvest model and 33% and 27% in 96-hour model, respectively. The results of the 24-model are depicted in FIG. 54 and the results of the 96-hour model are depicted in FIG. 55. The treatment groups include a medium control, 25 μM December 28 mM D/L Lac, and 10 μM LA.

Without being bound by theory, the data suggests that the inhibition of IL-1β-induced PGE₂ release in these models is similar for Dec and Dxp, regardless of the timing of drug administration, while treatment with Lac or LA serves better as a rescue drug to prevent cytokine-induced prostaglandin release.

### Example 20. Synergy Using a Combined Treatment of Hyaluronic Acid (HA) With Dex for the Transcription of MMP13

HA is a naturally occurring polysaccharide, found in connective tissues and synovial fluid, that serves to lubricate and cushion the joint. In osteoarthritis, HA injections are used as a viscosupplement to help replace degraded synovial fluid; thus, reducing pain and improving joint function. However, HA's effectiveness varies, with some patients experiencing significant relief while others see minimal benefit. Moreover, its effects can be short-lived, requiring repeated injections, and in rare situations, inflammatory reactions may occur.

Without being bound by theory, it is hypothesized that HA treatment in combination with Dex could provide a therapeutic strategy that promotes HA's beneficial effects while maintaining Dex's anti-inflammatory response. To investigate this, primary human chondrocytes from an osteoarthritic patient were treated with 0.1% HA and/or 0.1 μM Dex in monolayer culture or micromass pellets for 14 to 28 days. Total mRNA was then extracted, and qRTPCR was performed with primer pairs specific for MMP13 and the housekeeping gene GAPDH. Relative expression was then calculated versus controls, with each sample normalized to GAPDH amplification. When the two compounds were combined, suppression of MMP13 exceeded the expected responses. For example, in 14-day micromass cultures, MMP13 transcription was reduced by −1.6-fold and −38.6-fold compared to controls, respectively, for HA and Dex alone. However, when combined, there was a −7082.3-fold reduction observed (−40.2 additive or 61.8 multiplicative absolute value predicted). A similar response was seen in 28-day monolayer cultures with 1.0-fold, −3.6-fold, and −164.3-fold relative expressions for HA, Dex, and HA+Dex, respectively (−2.6 additive or 3.6 multiplicative absolute value expected). This data suggests that HA treatment may enhance the ability of Dex to suppress MMP13 transcription in these cultures.

TABLE 4
Representative MMP13 relative expression in micro mass, pellet culture
(note multiplicative values presented as absolute values).

| | Observed | Expected | Expected |
|---|---|---|---|
| | Relative | response | response |
| Treatment group | expression | additive | multiplicative |
| HA alone | −1.6 | | |
| Dex alone | −38.6 | | |
| HA + Dex | −7082.3 | −40.2 | 61.8 |

TABLE 5
Representative MMP13 relative expression in monolayer culture (note
multiplicative values presented as multiplied absolute values).

| | Observed | Expected | Expected |
|---|---|---|---|
| | Relative | response | response |
| Treatment group | expression | additive | multiplicative |
| HA alone | 1.0 | | |
| Dex alone | −3.6 | | |
| HA + Dex | −164.3 | −2.6 | 3.6 |

### Example 21. Synergy Using a Combined Treatment of DA with Try for the Transcription of Collagen 2a1 (Col2a1) and MMP13

Without being bound by theory, it is believed that fatty acid and Try metabolism play key roles in chondrocyte function and osteoarthritis progression, particularly in inflammation and cartilage homeostasis. Fatty acids influence chondrocyte energy production and inflammatory responses, with an imbalance in metabolism promoting inflammation and cartilage degradation. Moreover, Try metabolism generates bioactive metabolites, including kynurenine, which modulate immune responses and inflammation in the joint environment. Dysregulation of these pathways can contribute to osteoarthritis by promoting chronic inflammation and oxidative stress, accelerating cartilage breakdown. Targeting these metabolic pathways could offer new therapeutic approaches for osteoarthritis management.

It is hypothesized that DA treatment in combination with Try could stabilize chondrocyte metabolism and produce biological metabolites that promote chondrogenesis. To evaluate this, human osteoarthritic chondrocytes were treated with 0.5 mM DA and/or 1 mM Try in monolayer culture for up to 28 days with and without TGF-β3. Total mRNA was then isolated at the indicated times, and qRTPCR was performed with primers specific for MMP13, Col2a1, and the housekeeping gene GAPDH. Relative expression was then calculated versus controls, with normalization to GAPDH. When the two compounds were combined, it was observed that at certain time points, MMP13 inhibition or Col2a1 expression surpassed expectations. For example, MMP13 transcription was reduced by −1.1-fold, −2.6-fold, and −282.0-fold in 28-day cultures treated with DA, Try, or DA+Try, respectively (−3.6 additive or 2.86 multiplicative absolute value predicted). In contrast, 14-day cultures treated with DA, Try, or a combination of DA and Try in the presence of TGF-β3 exhibited relative Col2a1 mRNA levels of 26.5-fold, −1.4-fold, and 195.4-fold, respectively (25.1 additive or 37.1 multiplicative absolute value expected). This data suggests that combined treatment of these compounds may protect against catabolism while promoting anabolism.

TABLE 6
Representative MMP13 relative expression in 28-
day monolayer culture (note multiplicative values
presented as multiplied absolute values).

| | Observed | Expected | Expected |
|---|---|---|---|
| | Relative | response | response |
| Treatment group | expression | additive | multiplicative |
| DA alone | −1.1 | | |
| Try alone | −2.6 | | |
| DA + Try | −282.0 | −3.6 | 2.86 |

TABLE 7
Representative Col2a1 relative expression in 14-day
monolayer culture with TGF-β3 (note multiplicative
values presented as multiplied absolute values).

| | Observed | Expected | Expected |
|---|---|---|---|
| | Relative | response | response |
| Treatment group | expression | additive | multiplicative |
| DA alone | 26.5 | | |
| Try alone | −1.4 | | |
| DA + Try | 195.4 | 25.1 | 37.1 |

### Example 22. Methods for Lipidomics

To recapitulate the joint synovial membrane, a monolayer culture with human primary Fibroblast-Like Synoviocytes (HFLS) isolated from cadaver knees was used. HFLSs were cultured with media containing 1 ng/ml IL-1β and treatments for 6 hours to stimulate activation of inflammatory pathways. Treatment conditions included media control with no IL-1β, media control with IL-1β, 100μM DA, 200μM DA, 0.1 nM Dex, 100μM DA+0.1 nM Dex. After 6 hours all cell culture media was removed and HFLSs were then washed, harvested and processed for lipidomic analyses. Each sample was mixed with 34 deuterated oxylipin standards for quantification by ultra-high-pressure liquid chromatography coupled to mass spectrometry (UHPLC-MS). Analysis was performed using Metaboanalyst 6.0 and Prism GraphPad.

Oxylipin Quantification by UHPLC-MS was performed and samples were diluted with a cold solution of 5:3:2 MeOH:ACN:H2O (1 ml solution per 2.0e+6 cells). The samples were then vortexed vigorously for 30 minutes at 4° C., then centrifuged for 10 minutes at 18,213 rcf. Using 10 μL injection volumes, the supernatants were analyzed by ultra-high-pressure-liquid chromatography coupled to mass spectrometry (UHPLC-MS). Metabolites were resolved across a 1.7 μm, 2.1×100 mm Acquity BEH column using a 7-minute gradient and deuterated standard cocktails with known oxylipin compounds were run separately to determine retention times.

Tables 8-10 and FIG. 56 show the fold increases from the top 9 significant lipids that were modulated by DA. Significance is defined as p<0.1for this exploratory data set. Fold standard deviations (SDs) were calculated by propagating error from 4 biological replicates in each of the conditions and comparing to the positive IL-1β control (values greater than 1 indicate an increase from control).

TABLE 8
Fold increases from the top 9 significant
lipids that were modulated by DA.

| | Fold Changes | | |
|---|---|---|---|
| | | | Pos vs |
| | Pos vs | Pos vs | 100 uM |
| | 100 uM | 200 uM | DA + 0.1 |
| Compound | DA | DA | nM Dex |
| Maresin 2 | 5.7983 | 8.7081 | 5.5208 |
| 17(S)-HDHA | 5.1406 | 7.7065 | 5.0743 |
| 6-keto Prostaglandin F_{2α} | 3.2031 | 5.0949 | 3.4233 |
| 8-iso†Prostaglandin E₂/ | 2.4421 | 4.0172 | 2.7517 |
| Prostaglandin E₂/ | | | |
| Prostaglandin | | | |
| D₂ | | | |
| 5-iPF_{2α}-VI/ | 2.3301 | 3.8799 | 2.7727 |
| Prostaglandin F_{2α}/ | | | |
| Prostaglandin D₁/Prostaglandin | | | |
| E₁ | | | |
| 9(S)-HODE | 2.4459 | 3.7100 | 2.5376 |
| 13(S)-HODE | 2.2711 | 3.5820 | 2.6730 |
| Lipoxin A4/13-14-dihydro-15- | 1.8886 | 2.3706 | 2.1345 |
| keto Prostaglandin D₂ | | | |
| 11(S)-HETE/12(S)-HETE | 1.7647 | 2.1544 | 1.6155 |

TABLE 9
T-Test of fold increases from the top 9 significant
lipids that were modulated by DA.

| | T-Test | | |
|---|---|---|---|
| | | | Pos vs |
| | Pos vs | Pos vs | 100 uM |
| | 100 uM | 200 uM | DA + 0.1 |
| Compound | DA | DA | nM Dex |
| Maresin 2 | 0.0028 | 0.0246 | 0.0612 |
| 17(S)-HDHA | 0.0003 | 0.0176 | 0.0508 |
| 6-keto Prostaglandin F_{2α} | 0.0272 | 0.0571 | 0.1136 |
| 8-iso†Prostaglandin E₂/ | 0.0752 | 0.0872 | 0.1633 |
| Prostaglandin E₂/ | | | |
| Prostaglandin D₂ | | | |
| 5-iPF_{2α}-VI/ | 0.0392 | 0.0645 | 0.1288 |
| Prostaglandin F_{2α}/ | | | |
| Prostaglandin D₁/ | | | |
| Prostaglandin E₁ | | | |
| 9(S)-HODE | 0.0010 | 0.0198 | 0.0574 |
| 13(S)-HODE | 0.0062 | 0.0332 | 0.0649 |
| Lipoxin A4/13-14- | 0.0246 | 0.0554 | 0.0899 |
| dihydro-15-keto | | | |
| Prostaglandin D₂ | | | |
| 11(S)-HETE/12(S)- | 0.0234 | 0.0841 | 0.1047 |
| HETE | | | |

TABLE 10
Fold SDs of fold increases from the top 9 significant
lipids that were modulated by DA.

| | T-Test | | |
|---|---|---|---|
| | | | Pos vs |
| | Pos vs | Pos vs | 100 uM |
| | 100 uM | 200 uM | DA + 0.1 |
| Compound | DA | DA | nM Dex |
| Maresin 2 | 3.7716 | 7.1049 | 4.9820 |
| 17(S)-HDHA | 2.2586 | 5.0908 | 3.8656 |
| 6-keto Prostaglandin F_{1α} | 2.7257 | 5.0957 | 3.5764 |
| 8-iso†Prostaglandin E₂/ | 1.8129 | 3.6419 | 2.6135 |
| Prostaglandin E₂/ | | | |
| Prostaglandin D₂ | | | |
| 5-iPF_{2α}-VI/ | 1.4261 | 3.1129 | 2.3620 |
| Prostaglandin F_{2α}/ | | | |
| Prostaglandin D₁/ | | | |
| Prostaglandin E₁ | | | |
| 9(S)-HODE | 0.6694 | 1.8729 | 1.3966 |
| 13(S)-HODE | 0.7450 | 2.0045 | 1.5678 |
| Lipoxin A4/13-14- | 0.8063 | 1.3662 | 1.2923 |
| dihydro-15-keto | | | |
| Prostaglandin D₂ | | | |
| 11(S)-HETE/12(S)- | 0.6255 | 1.2156 | 0.7195 |
| HETE | | | |

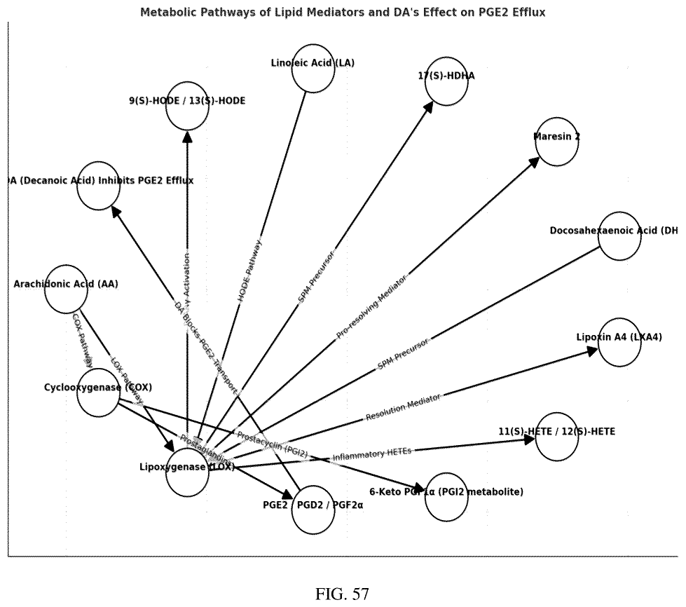

Without being bound by theory, molecular Pathways with DA's Inhibition of PGE₂ Efflux were analyzed and the following was observed, as summarized in the depiction of FIG. 57.

First, DA blocks PGE₂ efflux, leading to intracellular accumulation.

Despite IL-1β inducing PGE₂ synthesis, DA prevents its export, leading to a paradox of increased intracellular PGE₂ with reduced extracellular PGE2 (nearly baseline in supernatant) so PGE₂ cannot exert its paracrine effects through its receptors. This strongly suggests DA inhibits prostaglandin transporters, likely affecting MRP4 (ABCC4) (a key PGE₂ efflux transporter) and SLCO2A1 (prostaglandin transporter, PGT) a bidirectional transporter that also facilitates PGE₂ uptake.

The inhibition of efflux traps PGE₂ inside the cell, preventing its paracrine and autocrine signaling via EP receptors on neighboring cells.

Next, a shift toward specialized pro-resolving mediators (SPMs) was detected during the observation of the molecular Pathways associated with DA's Inhibition of PGE₂ Efflux. Specifically, the large increases in Maresin 2 (5.8-fold) and 17(S)-HDHA (5.1-fold) suggest that DA shifts lipid metabolism toward resolution-phase mediators, as further described with reference to Examples 23 and 24. With PGE₂ export blocked, the excess AA pool may be redirected to lipoxygenase (LOX) pathways, favoring (1) ALOX12/ALOX15 activation which leads to production of Maresin 2 & 17(S)-HDHA, and (2) SPM biosynthesis initiation, biasing the lipid mediator class switch toward resolution rather than inflammation.

Next, PGI₂ and other prostaglandins are still increased, and the continued increase indicates that: (1) the increase in 6-keto PGF_{1α} (3.2-fold)—a stable PGI₂ metabolite—suggests that prostacyclin biosynthesis is maintained despite PGE₂ transport being inhibited; (2) the elevated PGE₂-related species (e.g., 8-iso-PGE₂ PGE₂, PGD₂, prostaglandin F_{2α} (PGF_{2α})) indicate that COX-derived prostanoids are still being produced; and (3) because DA does not inhibit COX-2 or PLA2 directly, the effects appear post-synthetic, at the level of transport. Finally, PPARγ activation and lipoxygenase modulation indicates that the increased levels of 9(S)-HODE and 13(S)-HODE (˜2.3-2.4-fold) suggest that DA may activate PPARγ, a key anti-inflammatory nuclear receptor and that this activation likely promotes lipid homeostasis and dampens pro-inflammatory responses, aligning with the observed increases in pro-resolving mediators.

Without being bound by theory, it is believed that: (1) DA inhibits PGE₂ efflux, causing intracellular accumulation and reduced extracellular PGE₂, likely via MRP4 or SLCO2A1 inhibition, (2) the excess AA that would normally be used for secreted PGE₂ is redirected toward pro-resolving lipid mediators (Maresin 2, 17(S)-HDHA, Lipoxin A4), (3) PPARγ activation (via increased 9/13-HODE) further supports a shift toward resolution-phase signaling, (4) despite blocking PGE₂ efflux, DA does not inhibit COX-2 or PLA2 activity, allowing continued prostaglandin production, and (5) the maintenance of PGI₂ synthesis suggests that endothelial and vascular homeostasis remain intact.

This suggests DA shifts inflammatory lipid signaling away from PGE₂-driven inflammation toward pro-resolving pathways, which could have therapeutic implications in inflammatory diseases where excessive PGE₂ secretion drives pathology.

Regarding AA-Derived Pathways that include prostaglandins and HETEs, AA is metabolized by cyclooxygenases (COX) and lipoxygenases (LOX) to generate inflammatory prostaglandins, thromboxanes, and hydroxy-eicosatetraenoic acids (HETEs). Some key AA-Derived Lipids include PGE₂, PGD₂, and prostaglandin F_{2α} (PGF_{2α}) and these AA-Derived Lipids are synthesized via Cyclooxygenase-1 (COX-1)/COX-2 to PGH₂ to PGE₂, PGD₂, PGF_{2α}. PGE₂ is inflammatory but also regulates immune responses and pain. PGD₂ has dual roles: inflammatory in early response but promotes resolution in late phases. PGF_{2α} is involved in vasoconstriction and smooth muscle contraction.

6-Keto prostaglandin F_{1α} (PGI₂ metabolite) is a stable breakdown product of PGI₂, and PGI₂ is a vasodilatory and anti-thrombotic mediator synthesized via COX to PGH₂ to PGI₂ to counterbalance the pro-inflammatory effects of PGE₂.

11(S)-HETE/12(S)-HETE is formed via 12-LOX and 11-LOX pathways from AA. Specifically, 12(S)-HETE is associated with platelet activation and inflammation, and 11(S)-HETE may contribute to vascular homeostasis.

Regarding docosahexaenoic Acid (DHA)-Derived Pathways including Specialized Pro-Resolving Mediators (SPMs), DHA is metabolized by 12/15-LOX pathways to generate pro-resolving lipid mediators (SPMs) like maresins and protectins, which counteract inflammation. In some embodiments, DHA-Derived Lipids may include Maresin 2 and 17(S)-HDHA. Maresin 2 is derived from DHA via 12-LOX/15-LOX and is strongly pro-resolving, promoting tissue repair and inhibiting neutrophil recruitment. Similarly, 17(S)-HDHA is an intermediate in maresin, protectin, and resolvin biosynthesis and is derived from DHA oxidation via 15-LOX. 17(S)-HDHA is important in switching macrophages to a pro-resolving state.

Regarding, Linoleic Acid (LA)-Derived Pathways including hydroxy-octadecadienoic acids (HODEs), LA is metabolized into HODEs, which have roles in inflammation and lipid metabolism. In some embodiments, key LA-Derived Lipids include 9(S)-HODE and 13(S)-HODE. Both 9(S)-HODE and 13(S)-HODE are formed from LA via 15-LOX and 12-LOX, are activators of PPARγ, which promotes anti-inflammatory and metabolic functions, and are involved in resolving inflammation, lipid metabolism, and oxidative stress responses.

Finally, the pathway interactions include: (1) AA metabolism pathway (COX pathway) which produces prostaglandins (PGE₂, PGD₂, PGF_{2α}) which inflammatory but also regulates immune responses and has homeostatic roles; (2) DHA metabolism pathway (LOX pathway) which produces Pro-resolving mediators (Maresin 2, 17(S)-HDHA); (3) Linoleic acid metabolism pathway which produces 9(S)-HODE, 13(S)-HODE (PPARγ activation, anti-inflammatory); (4) Lipoxin A4 formation pathway that bridges AA-derived and DHA-derived pathways by resolving inflammation; and (5) DA inhibits PGE₂ efflux pathway which redirects AA metabolism toward resolution-phase lipids rather than secreted inflammatory prostanoids.

### Example 23. DA Enhances Maresin 2 and Resolvins, Inhibits Prostaglandin Efflux, Modulates Membrane Signaling, and Synergizes With Low-Dose Dex for Inflammation Resolution and Pain Relief

Inflammation resolution is a critical process for tissue homeostasis, yet persistent inflammation contributes to chronic pain and impaired healing in diseases such as osteoarthritis and rheumatoid arthritis. DA, a saturated medium-chain fatty acid, is a novel anti-inflammatory, pro-resolving agent that influences proinflammatory cytokine and lipid mediator signaling. In osteoarthritis, chronic inflammation involves proinflammatory cytokines such as IL-1β that activate NF-Kb resulting in COX-2-mediated synthesis of PGE₂ and increased cartilage destruction by MMP proteins. Resolution of inflammation and restoration of tissue homeostasis involves specialized pro-resolving lipid mediators (SPMs) that promote anti-inflammatory and pro-resolving microenvironments.

Lipid mediator metabololipidomics showed that two SPMs, Maresin 2 (MaR2) and 17(S)-HDHA, a precursor to 17(S)-resolvins, increase in IL-1β-stimulated synoviocytes when treated with DA. Moreover, DA increased intracellular accumulation of PGE₂ and other prostaglandin metabolites suggesting that DA may inhibit PGE₂ & prostaglandin efflux transporters, like MRP4, resulting in the observed significant reductions of extracellular prostaglandin levels in IL-1β-stimulated cells. Notably, DA does not affect COX-2 or cPLA2 expression or activity, suggesting that in addition to its effects on prostaglandin efflux, increased cytosolic AA may shunt into maresin and resolvin biosynthesis pathways. MaR2 and resolvins inhibit NLRP3 inflammasome activation which is consistent with the ability of DA to decrease inflammasome-mediated release of IL-1β. The resolvin metabolite, 17(S)-HDHA is associated with decreased pain in osteoarthritic patients; thus, both pain and inflammation may decrease with DA treatment. Beyond its biochemical effects, DA may directly modulate membrane fluidity by integrating into cellular membranes as a free fatty acid or as a phospholipid component (phosphatidylcholine, phosphatidylethanolamine), leading to increased membrane rigidity. This biophysical change may influence membrane-associated receptor signaling, lipid raft formation, and cellular responsiveness to inflammatory stimuli, further contributing to its anti-inflammatory properties. This dual mechanism-enhancing pro-resolving MaR2 and 17(S)-HDHA while sequestering PGE₂ and other prostaglandins intracellularly-suppresses NF-κB and inflammasome activation and reduces MMP13 expression, ultimately decreasing inflammation-driven matrix degradation. Functionally, increased MaR2 levels enhance tissue repair pathways by promoting macrophage polarization toward an M2 phenotype and downregulating TGF-β1, protecting against pathological fibrosis.

Additionally, DA mitigates pain sensitization by suppressing Naᵥ1.7 expression and reducing its phosphorylation, thereby increasing the threshold for activation and reducing nociceptive sodium currents, suggesting a direct role in pain resolution. Furthermore, we propose that DA, when combined with a very low dose of Dex, may provide synergistic anti-inflammatory effects while reducing the adverse effects of steroids. While Dex broadly suppresses inflammation via glucocorticoid receptor-mediated transcriptional regulation, DA complements this by enhancing SPM synthesis (MaR2 and 17(S)-HDHA), selectively inhibiting prostaglandin efflux, and modifying membrane signaling. This synergy could allow for a significant reduction in the required steroid dose, minimizing steroid-associated side effects such as immunosuppression and tissue catabolism while enhancing inflammation resolution and promotion of healing. These findings identify DA as a multifunctional therapeutic agent that combines mechanisms of anti-inflammation and pro-resolution to reduce pain, promote healing, and enhance the efficacy of low-dose glucocorticoid therapy. Further in vivo studies will determine its translational potential in clinical applications for osteoarthritis, rheumatoid arthritis, and other chronic inflammatory conditions.

### Example 24. Formation, Anti-Inflammatory Properties, and Therapeutic Potential of Maresin 2

Maresin 2 (MaR2, 13R, 14S-dihydroxy-docosahexaenoic acid) is a specialized pro-resolving lipid mediator (SPM) derived from docosahexaenoic acid (DHA, 22:6, n-3) and plays a crucial role in inflammation resolution, tissue repair, and immune modulation, distinguishing itself from traditional anti-inflammatory drugs that only suppress inflammation without actively promoting resolution.

MaR2 is primarily synthesized in macrophages from DHA, which is released from membrane phospholipids by phospholipase A2 (cPLA2) in response to inflammatory signals. The 12-lipoxygenase (12-LOX, ALOX12) enzyme catalyzes the oxygenation of DHA, forming the intermediate 13S,14S-epoxy-maresin, which is subsequently converted into MaR2 via enzymatic hydrolysis. This pathway ensures that MaR2 is produced at sites of inflammation, where it can exert its biological effects.

Unlike conventional anti-inflammatory drugs that block cytokine production or cyclooxygenase activity, MaR2 actively shifts immune cells from a pro-inflammatory to a resolution state. Specifically, MaR2 suppresses Pro-Inflammatory Cytokines by inhibiting NF-κB activation, leading to decreased expression of IL-1β, TNF-α, and IL-6, key mediators of chronic inflammation. Additionally, MaR2 suppresses Pro-Inflammatory Cytokines reduces COX-2 expression and PGE₂ production, limiting inflammatory signaling. MaR2 also promotes Macrophage Polarization and Efferocytosis by enhancing macrophage polarization toward the M2 phenotype, which is associated with tissue repair and anti-inflammatory activity. Specifically, MaR2 stimulates efferocytosis, the process by which macrophages clear apoptotic cells, preventing the persistence of inflammation. MaR2 also regulates Extracellular Matrix (ECM) Remodeling by modulating matrix metalloproteinases (MMPs), particularly MMP13, balancing ECM degradation and tissue repair. MaR2 upregulates TGF-β1, promoting fibroblast function and collagen deposition. Finally, MaR2 modulate pain and neuroinflammation by reducing pain hypersensitivity by suppressing Naᵥ1.7 expression and phosphorylation, increasing the threshold for nociceptive activation. MaR2 inhibits glial activation in the central nervous system, preventing neuroinflammatory pain amplification.

MaR2 holds promise for treating chronic inflammatory conditions, including rheumatoid arthritis, osteoarthritis, neuroinflammatory disorders, and cardiovascular diseases. Unlike steroids or NSAIDs, which can have adverse effects on immune function and tissue integrity, MaR2 actively resolves inflammation without compromising host defense mechanisms. Furthermore, MaR2 may synergize with low-dose glucocorticoids, enhancing therapeutic efficacy while reducing steroid-associated side effects.

Thus, MaR2 is an endogenous pro-resolving mediator that may be used to control inflammation, promote tissue healing, and reduce pain. Specifically, MaR2 modulates cytokine production, enhances macrophage function, regulates ECM remodeling, and alters pain pathways.

Articles such as “the,” “a,” and “an” can connote the singular or plural. Also, the word “or” when used without a preceding “either” (or other similar language indicating that “or” is unequivocally meant to be exclusive—e.g., only one of x or y, etc.) shall be interpreted to be inclusive (e.g., “x or y” means one or both x or y).

As used in this disclosure, the term “or” is defined as a logical disjunction (i.e., and/or) and does not indicate an exclusive disjunction unless expressly indicated as such with the terms “either,” “unless”, “alternatively”, and words of similar effect.

As used herein, and unless otherwise indicated, the term “approximately” refers to an acceptable error for a particular value as determined by those of ordinary skill in the art, which depends in part on how it is measured or value is determined. In certain contexts, the term “approximately” refers to within 1, 2, 3, or 4 standard deviations. In certain contexts, the term “approximately” refers to within 50%, 20%, 15%, 10%, 9%, 8%, 7%, 6%, 5%, 4%, 3%, 2%, 1%, 0.5% or 0.05% of a given value or range.

The term “and/or” shall also be interpreted to be inclusive (e.g., “x and/or y” means one or both x or y). In situations where “and/or” or “or” are used as a conjunction for a group of three or more items, the group should be interpreted to include one item alone, all the items together, or any combination or number of the items.

The terms have, having, include, and including should be interpreted to be synonymous with the terms comprise and comprising. The use of these terms should also be understood as disclosing and providing support for narrower alternative embodiments where these terms are replaced by “consisting” or “consisting essentially of.”

Unless otherwise indicated, all numbers or expressions, such as those expressing dimensions, physical characteristics, and the like, used in the specification (other than the claims) are understood to be modified in all instances by the term “approximately.” At the very least, and not as an attempt to limit the application of the doctrine of equivalents to the claims, each numerical parameter recited in the specification or claims which is modified by the term “approximately” should be construed in light of the number of recited significant digits and by applying ordinary rounding techniques.

All disclosed ranges are to be understood to encompass and provide support for claims that recite any and all subranges or any and all individual values subsumed by each range. For example, a stated range of 1 to 10 should be considered to include and provide support for claims that recite any and all subranges or individual values that are between and/or inclusive of the minimum value of 1 and the maximum value of 10; that is, all subranges beginning with a minimum value of 1 or more and ending with a maximum value of 10 or less (e.g., 5.5 to 10, 2.34 to 3.56, and so forth) or any values from 1 to 10 (e.g., 3, 5.8, 9.9994, and so forth).

All disclosed numerical values are to be understood as being variable from 0-100% in either direction and thus provide support for claims that recite such values or any and all ranges or subranges that can be formed by such values. For example, a stated numerical value of 8should be understood to vary from 0 to 16 (100% in either direction) and provide support for claims that recite the range itself (e.g., 0 to 16), any subrange within the range (e.g., 2 to 12.5) or any individual value within that range (e.g., 15.2).

The terms recited in the claims should be given their ordinary and customary meaning as determined by reference to relevant entries in widely used general dictionaries and/or relevant technical dictionaries, commonly understood meanings by those in the art, etc., with the understanding that the broadest meaning imparted by any one or combination of these sources should be given to the claim terms (e.g., two or more relevant dictionary entries should be combined to provide the broadest meaning of the combination of entries, etc.) subject only to the following exceptions: (a) if a term is used in a manner that is more expansive than its ordinary and customary meaning, the term should be given its ordinary and customary meaning plus the additional expansive meaning, or (b) if a term has been explicitly defined to have a different meaning by reciting the term followed by the phrase “as used in this document shall mean” or similar language (e.g., “this term means,” “this term is defined as,” “for the purposes of this disclosure this term shall mean,” etc.). References to specific examples, use of “i.e.,” use of the word “invention,” etc., are not meant to invoke exception (b) or otherwise restrict the scope of the recited claim terms. Other than situations where exception (b) applies, nothing contained in this document should be considered a disclaimer or disavowal of claim scope.

The subject matter recited in the claims is not coextensive with and should not be interpreted to be coextensive with any embodiment, feature, or combination of features described or illustrated in this document. This is true even if only a single embodiment of the feature or combination of features is illustrated and described in this document.

1. A pharmaceutical composition, comprising:
dexamethasone at a concentration of at least 400 picomolar and up to 20 millimolar; and
decanoic acid at a concentration of at least 10 micromolar and up to 5 millimolar.

2. The pharmaceutical composition of claim 1, wherein the dexamethasone has a concentration of at least 800 picomolar and up to 800 nanomolar.

3. The pharmaceutical composition of claim 1, wherein the dexamethasone has a subclinical dose amount of at least 100 micrograms and up to 2.5 milligrams.

4. The pharmaceutical composition of claim 1, wherein the decanoic acid has a concentration of at least 10 micromolar and up to 1 millimolar.

5. The pharmaceutical composition of claim 1, further comprising water, wherein:
a majority of the dexamethasone is dissolved in the water; and
a majority of the decanoic acid is dissolved in the water.

6. The pharmaceutical composition of claim 1, further comprising a lipid mediator.

7. The pharmaceutical composition of claim 6, wherein the lipid mediator comprises 13R,14S-dihydroxy-docosahexaenoic acid.

8. The pharmaceutical composition of claim 1, further comprising one or more amino acids.

9. The pharmaceutical composition of claim 5, wherein the one or more amino acids comprises tryptophan.

10. The pharmaceutical composition of claim 1, wherein the dexamethasone is selected from dexamethasone esters, wherein the esters are selected from dexamethasone phosphate, dexamethasone sulfate, dexamethasone acetate, dexamethasone propionate, dexamethasone valerate, dexamethasone pivalate, dexamethasone tert-butylacetate, dexamethasone succinate, dexamethasone troxundate, dexamethasone 17-propionate, dexamethasone dipropionate, dexamethasone metasulphobenzoate, dexamethasone isonicotinate, dexamethasone diethylaminoacetate, dexamethasone acefurate, dexamethasone cipecilate, dexamethasone octanoate, dexamethasone decanoate, dexamethasone palmitate, and dexamethasone linoleate.

11. The pharmaceutical composition of claim 1, further comprising a hydrogel, wherein:
the hydrogel comprises the water and hyaluronic acid; and
the hyaluronic acid has a molecular weight of at least 500 kilodaltons.

12. A method to treat osteoarthritis, comprising:
providing a pharmaceutical composition comprising a therapeutically-effective amount of dexamethasone and a therapeutically-effective amount of decanoic acid; and
administering the pharmaceutical composition to an osteoarthritic joint of a patient.

13. The method of claim 12, wherein:
the therapeutically-effective amount of dexamethasone is a subclinical dose of the dexamethasone.

14. The method of claim 12, wherein the therapeutically-effective amount of dexamethasone and the therapeutically-effective amount of decanoic acid are synergistically effective to treat the osteoarthritic joint.

15. The method of claim 14, wherein the therapeutically-effective amount of dexamethasone and the therapeutically-effective amount of decanoic acid are synergistically effective to reduce inflammasome-mediated inflammation in the osteoarthritic joint.

16. The method of claim 14, wherein the therapeutically-effective amount of dexamethasone and the therapeutically-effective amount of decanoic acid are synergistically effective to reduce interleukin-1beta (IL-1β) concentration and prostaglandin E₂ (PGE₂) in the osteoarthritic joint.

17. The method of claim 14, wherein the therapeutically-effective amount of dexamethasone and the therapeutically-effective amount of decanoic acid are synergistically effective to increase collagen, type II, alpha 1 (Col2a1) in the osteoarthritic joint.

18. The method of claim 12, wherein the therapeutically-effective amount of the dexamethasone and the therapeutically-effective amount of the decanoic acid inhibit IL-1β-induced PGE₂ release from normal human chondrocytes.

19. The method of claim 12, wherein the therapeutically-effective amount of the dexamethasone and the therapeutically-effective amount of the decanoic acid have an increased actual relative potency as compared to an expected relative potency at inhibiting IL-1β-induced PGE2 release from normal human chondrocytes.

20. The method of claim 19, wherein the actual relative potency is at least 6% greater than the expected relative potency at inhibiting IL-1β-induced PGE₂ release from normal human chondrocytes when a concentration of the therapeutically-effective amount of the decanoic acid is approximately 2.0 micromolar.

21. The method of claim 19, wherein the actual relative potency is at least 34% greater than the expected relative potency at inhibiting IL-1β-induced PGE2 release from normal human chondrocytes when a concentration of the therapeutically-effective amount of the decanoic acid is approximately 12.5 micromolar.

22. The method of claim 19, wherein the actual relative potency is at least 150% greater than the expected relative potency at inhibiting IL-1β-induced PGE2 release from normal human chondrocytes when a concentration of the therapeutically-effective amount of the decanoic acid is approximately 50.0 micromolar.

23. The method of claim 12, wherein the therapeutically-effective amount of the dexamethasone is selected from dexamethasone esters, wherein the esters are selected from dexamethasone phosphate, dexamethasone sulfate, dexamethasone acetate, dexamethasone propionate, dexamethasone valerate, dexamethasone pivalate, dexamethasone tert-butylacetate, dexamethasone succinate, dexamethasone troxundate, dexamethasone 17-propionate, dexamethasone dipropionate, dexamethasone metasulphobenzoate, dexamethasone isonicotinate, dexamethasone diethylaminoacetate, dexamethasone acefurate, dexamethasone cipecilate, dexamethasone octanoate, dexamethasone decanoate, dexamethasone palmitate, and dexamethasone linoleate.

24. The method of claim 12, wherein the pharmaceutical composition comprises the therapeutically-effective amount of the dexamethasone at a concentration of at least 400 picomolar and up to 20 millimolar.

25. The method of claim 12, wherein the pharmaceutical composition comprises a subclinical dose of the therapeutically-effective amount of the dexamethasone of at least 100 micrograms and up to 2.5 milligrams.

26. The method of claim 12, wherein the pharmaceutical composition comprises the therapeutically-effective amount of the decanoic acid at a concentration of at least 10 micromolar and up to 1 millimolar.

27. The method of claim 12, wherein the pharmaceutical composition comprises water.

28. The method of claim 27, wherein:
a majority of the therapeutically-effective amount of the dexamethasone is dissolved in the water; and
a majority of the therapeutically-effective amount of the decanoic acid is dissolved in the water.

29. The method of claim 27, wherein:
the pharmaceutical composition comprises a hydrogel;
the hydrogel comprises hyaluronic acid that has a molecular weight of at least 500 kilodaltons, the water, at least a portion of the therapeutically-effective amount of the dexamethasone, and at least a portion of the therapeutically-effective amount of the decanoic acid; and
the hyaluronic acid has a molecular weight of at least 500 kilodaltons.

30. The method of claim 12, wherein the administering comprises injecting the pharmaceutical composition into or adjacent to the osteoarthritic joint.

31. The method of claim 12, wherein the pharmaceutical composition further comprises a lipid mediator.

32. The method of claim 31, wherein the lipid mediator comprises 13R,14S-dihydroxy-docosahexaenoic acid.

33. The method of claim 12, wherein the pharmaceutical composition further comprises one or more amino acids.

34. The method of claim 33, wherein the one or more amino acids comprises tryptophan.
